SEQUENCE LISTING (2 sequences)

SEQ ID NO: 1 - DNA, 20 bp, synthetic construct
  source 1..20: mol_type = other DNA
tccccgtagagattacaatg

SEQ ID NO: 2 - DNA, 20 bp, synthetic construct
  source 1..20: mol_type = other DNA
cttgtggcaatgacaaattg

(19) United States
(12) Patent Application Publication
(10) Pub. No.: US 2024/0216425 A1
(43) Pub. Date: Jul. 4, 2024

(54) Engineered Macrophages for Cancer Immunotherapy

(71) Applicant: The Trustees of the University of Pennsylvania, Philadelphia, PA (US)

(72) Inventors: Dennis E. Discher, Philadelphia, PA (US); Jason Christopher Andrechak, Philadelphia, PA (US); Lawrence Dooling, Philadelphia, PA (US)

(21) Appl. No.: 18/400,822

(22) Filed: Dec. 29, 2023

Related U.S. Application Data
(60) Provisional application No. 63/477,891, filed on Dec. 30, 2022.

(51) Int. Cl. A61K 35/15; A61K 39/00; A61P 35/00; C07K 14/705; C07K 16/40; C12N 5/0786; C12N 9/22; C12N 15/11; C12N 15/86

(52) U.S. Cl. CPC A61K 35/15; A61K 39/4614; A61K 39/464402; A61K 39/464456; A61P 35/00; C07K 14/705; C07K 16/40; C12N 5/0645; C12N 9/22; C12N 15/11; C12N 15/86; A61K 2239/13; C12N 2310/20; C12N 2740/15043

(57) ABSTRACT

The present invention relates to compositions and methods that provide modified phagocytes (e.g., macrophages) or precursor cells thereof useful for the treatment of cancer in subjects in need thereof.

### CROSS-REFERENCE TO RELATED APPLICATION

The present application claims priority under 35 U.S.C. § 119(e) to U.S. Provisional Patent Application No. 63/477,891, filed Dec. 30, 2022, which is hereby incorporated by reference in its entirety herein.

### STATEMENT REGARDING FEDERALLY SPONSORED RESEARCH OR DEVELOPMENT

This invention was made with government support under HL124106 awarded by the National Institutes of Health. The government has certain rights in the invention.

### SEQUENCE LISTING

The present application contains a Sequence Listing which has been submitted in XML format via Patent Center and is hereby incorporated by reference in its entirety. Said XML file, created on Dec. 29, 2023, is named “046483-7416US1(03411) Sequence Listing ST_26.xml” and is 2,900 bytes in size.

### BACKGROUND

Monoclonal antibodies (mAbs) are a major focus of the biopharma industry as epitope-specific therapeutics against a wide range of diseases. As such, more than one-hundred distinct mAbs have achieved FDA approval for treatment of various diseases and disorders. Such approvals highlight the capacity for mAb development as effective therapies, including against cancer. mAbs currently in the clinic for cancer include those that bind lineage-restricted antigens (e.g. anti-CD20, Rituximab) and those that function as immune checkpoint inhibitors particularly involving T cells (e.g. anti-PD1 and anti-PDL1). Further opportunities potentially include identification of anti-tumor IgG that synergize with blockade of other immune system checkpoints. Such synergy could address the failure of some otherwise promising mAbs to show anti-tumor efficacy, especially against solid tumors.

Macrophage engulfment of other cells and microbes help to maintain tissue homeostasis and provides a first line of immune defense. Within solid tumors, the phagocytic forces exerted by macrophages must exceed the mechanical strength of the cohesion between solid tumor cells. A large imbalance of such cell-cell interactions has long been seen to drive ‘phase separation’ in mixtures of diverse tissue cell systems. Intriguingly, clusters of macrophages in tumor nests have been correlated with patient survival for at least two solid tumor types, and macrophage aggregation in the contexts of tissue injury has been compared to platelet clots, suggesting collective mechanisms. On the other hand, tumor-associated macrophages more typically correlate with poor clinical prognoses, and such tumor-associated macrophages (TAMs) not only promote growth and invasion in some cancers, as well as mediate immune evasion and tolerance, but also often lack phagocytic function.

Systemic injections of highly phagocytic, bone marrow derived monocytes/macrophages have proven ineffective in trials against solid tumors. Furthermore, injection of SIRPα-knockdown macrophages has already been reported to promote growth of solid tumors despite the likely presence of tumor-enriched opsonins that activate phagocytosis by TAMs.

Therefore, there is a great need in the art for identifying novel anti-tumor therapies and novel methods for enhancing the activity of phagocytes such as macrophages to specifically target tumor cells. The present invention addresses this need.

### SUMMARY OF THE DISCLOSURE

As described herein, the present disclosure relates to compositions and methods that provide modified phagocytes (e.g., macrophages) or precursor cells thereof useful for the treatment of cancer in subjects in need thereof.

In one aspect, the disclosure provides a modified phagocytic cell or precursor cell thereof comprising:
a. an isolated nucleic acid encoding a fusion protein comprising an estrogen receptor (ER) ligand binding domain linked to a Hoxb8 protein,
b. a modified endogenous genetic locus encoding a SIRPα protein; and
c. Fc-γR receptors bound to at least one targeting antibody specific for an antigen on a target cell.

In certain embodiments, the phagocytic cell is a macrophage.

In certain embodiments, the precursor cell is a monocyte precursor cell.

In certain embodiments, the modified SIRPα locus reduces or eliminates expression of SIRPα protein.

In certain embodiments, the target cell antigen is a tumor-associated antigen.

In certain embodiments, the target cell is a tumor cell.

In certain embodiments, the isolated nucleic acid is introduced into the phagocytic cell or precursor cell thereof via a lentiviral transduction system.

In certain embodiments, the modified locus encoding a SIRPα protein is modified via a CRISPR system.

In certain embodiments, the nucleic acid encoding the ER-Hoxb8 protein is introduced prior to the modification of the SIRPα protein.

In certain embodiments, expression of the ER-Hoxb8 fusion protein reversibly or conditionally immortalizes the phagocytic cell or precursor cell thereof when the cell is contacted with a ligand of the estrogen receptor.

In certain embodiments, the macrophage or precursor cell thereof is of human origin. In another aspect, the disclosure provides a method of producing a modified phagocytic cell or precursor cell thereof, comprising:
a. obtaining a precursor cell from a subject;
b. transducing the precursor cell with a expression vector encoding a fusion protein comprising an estrogen receptor ligand binding domain and a Hoxb8 protein;
c. contacting the precursor cell with a estrogen receptor ligand to reversibly immortalize the precursor cell;
d. modifying an endogenous genetic locus encoding a SIRPα protein;
e. terminally differentiating the precursor cell into a phagocytic cell by withdrawal of the ER ligand and culture in the presence of an effective amount of stem cell factor (SCF) and granulocyte-macrophage colony-stimulating factor (GM-CSF); and
f. coating the Fc receptors of the phagocytic cell with at least one targeting antibody specific for an antigen on a target cell.

In certain embodiments, the modification to the genetic locus encoding SIRPα reduces or eliminates expression of SIRPα protein.

In certain embodiments, the genetic modification is accomplished using a CRISPR knockout system.

In certain embodiments, the precursor cell is a monocyte precursor cell.

In certain embodiments, the phagocytic cell is a macrophage.

In certain embodiments, the expression vector is a lentiviral vector.

In certain embodiments, the precursor cell is obtained from bone marrow.

In certain embodiments, the estrogen receptor ligand is β-estradiol.

In certain embodiments, the target cell is a tumor cell.

In certain embodiments, the target cell antigen is a tumor-associated antigen.

In certain embodiments, the subject is a mammal.

In certain embodiments, the subject is a human.

In another aspect, the disclosure provides a method of treating cancer in a subject in need thereof, comprising administering to the subject an effective amount of the modified phagocyte or precursor cell thereof of any one of claims 1-11 thereby treating the cancer, wherein the targeting antibody is specific for at least one antigen associated with the cancer.

In certain embodiments, the method of the above aspects or embodiments or any aspect or embodiment disclosed herein further comprises administering to the patient an effective amount of an opsonin specific for the cancer.

In certain embodiments, the opsonin is the targeting antibody.

In certain embodiments, the opsonin is different from the targeting antibody.

In certain embodiments, the subject is a mammal.

In certain embodiments, the subject is a human.

In certain embodiments, the cancer is selected from the group consisting of brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, and uterine cancer.

In certain embodiments, the cancer is melanoma.

In certain embodiments, the target cell antigen is Tyrp1.

In another aspect, the disclosure provides a composition comprising the modified phagocyte or precursor cell thereof of any one of the above aspects or embodiments or any aspect or embodiment disclosed herein and a pharmaceutically acceptable carrier or excipient.

### BRIEF DESCRIPTION OF THE DRAWINGS

The following detailed description of preferred embodiments of the invention will be better understood when read in conjunction with the appended drawings. For the purpose of illustrating the invention, there are depicted in the drawings certain embodiments of the invention. It should be understood, however, that the invention is not limited to the precise arrangements and instrumentalities of the embodiments shown in the drawings.

FIGS. 1A-1G illustrate that macrophages cooperatively phagocytose IgG-opsonized cancer cells in engineered tumoroids. FIG. 1A Engineered ‘immuno-tumoroid’ model for phagocytosis of cohesive tumor cells. Tumoroids were formed by culturing B16 melanoma cells on non-adhesive surfaces and subsequently adding bone marrow-derived macrophages with or without opsonizing anti-Tyrp1 IgG. The inset depicts a macrophage-melanoma tumoroid ‘phagocytic synapse’ with FcR-IgG signaling that promotes phagocytosis and CD47: SIRPα that inhibits phagocytosis. FIG. 1B Micropipette aspiration of B16 tumoroids at t=0 and 1=10 min of constant stress (aspiration pressure, ΔP). Scale bar: 50 μm. Tumoroid strain or creep is plotted vs. time and fit with a standard linear model to determine the elastic moduli and viscosity. The inset depicts the first 90s of the creep experiment. Forces on the order of 100 nN are predicted to be sustained by tumoroids without rupture based on typical aspiration pressures. FIG. 1C Phagocytosis of CD47 KO and WT B16 tumoroids ˜18 h after addition of macrophages at a nominal 1:1 macrophage:B16 ratio with or without anti-Tyrp1. The % phagocytic macrophages corresponds to GFP+ macrophages in cell suspensions from disaggregated tumoroids (mean±SD, n=3 where each replicate consists of cells pooled from the same 96-well plate). Statistical significance was assessed by two-way ANOVA and Tukey's multiple comparison test. FIG. 1D Opsonization with anti-Tyrp1 limits the growth of CD47 KO ‘immuno-tumoroids’, but not WT immuno-tumoroids. (i) Representative fluorescence images depict growth or repression of CD47 KO (green) in immuno-tumoroids from days 1˜4 when untreated (top row) or treated with anti-Tyrp1 (bottom row). Macrophages (magenta, ˜1:1 ratio to initial B16 number) and anti-Tyrp1 were added immediately after the day 1 images were acquired. Scale bar: 0.5 mm. Confocal z-slices of CD47 immuno-tumoroids on day 2 clearly depict B16 cells engulfed by macrophages (white arrows indicate engulfed cell). Engulfment events were considered to be cellular phagocytosis if the internalized GFP signal colocalized with DNA signal from an internalized nucleus (mean±SD, n=3 tumoroids). (ii) Tumoroid growth was measured by calculating the projected GFP+ area at indicated time points (mean±SD, n=6 tumoroids). Statistical significance was assessed by the Mann-Whitney test (unpaired, two-tailed, * p=0.03, ** p=0.0043) comparing GFP+ areas with and without anti-Tyrp1 at each time point. Solid lines are nonlinear regression of the data to a simple exponential of the form A(t)=A1 ek(t−1). The doubling time τd or half-life τ½ was calculated from the fitted rate constant k. FIG. 1E A reaction-kinetic model accounts for division of cancer cells (A) in tumoroids and phagocytosis by n macrophages (M). Exponential growth or decay of tumoroid area (fitted curves) depends on the ratio of macrophages to B16 cells as well as the presence of CD47 on target cells (left) (mean±SEM, n=6-8 tumoroids from a representative of four or more experiments). The effective growth rate (keff) from fitted exponentials of CD47 KO tumoroids exhibits a Hill-like dependence on macrophage number (Hill parameter=−1.8, IC50=3.1, mean±SEM, n≥24 tumoroids from four independent experiments). A constrained fit of WT tumoroid growth with the same Hill parameter indicates much higher macrophage:B16 ratios are required for tumoroid elimination (IC50=˜15) (right). FIG. 1F (i) Effective growth rates of WT tumoroids with anti-CD47 and anti-Tyrp1 also show Hill-like dependence on macrophage number (Hill parameter=−2.7, IC50 ratio=2.3, mean±SEM, n≥18 tumoroids across three independent experiments). The concentration of anti-CD47 (133 nM) used is nearly saturating for CD47 on WT B16 as shown in FIG. 8A-ii. (ii) The effective growth rate of WT tumoroids at a fixed 3:1 macrophage:B16 ratio depends on the concentration of anti-CD47, but tumoroid elimination is not cooperative with respect to blocking Ab level (mean±SEM, n≥16 tumoroids across three independent experiments). Statistically significant growth repression (closed symbols, keff<0, one-sample t-test) is only observed at the highest level of blocking (>100 nM anti-CD47 as used in Fig F-i) that exceeds by ˜4-fold the measured affinity of blocking Ab for CD47 (K ˜25 nM as shown in FIG. 8A-ii). The effective growth rate of CD47 KO tumoroids at a fixed 3:1 macrophage:B16 ratio also depends on the concentration of anti-Tyrp1 but is not cooperative with respect to opsonizing Ab level (mean±SEM, n=8 tumoroids representative of two independent experiments). Statistically significant growth repression (closed symbols, keff<0, one-sample t-test) is observed at sub-saturating levels of anti-Tyrp1 (K˜47 nM as shown in FIG. S2A-ii).

FIG. 1G Proposed cooperative tug-of-war by maximally phagocytic macrophages to (i) disrupt target cancer cell adhesions and subsequently (ii) phagocytose individual cells that are no longer attached to neighbors.

FIGS. 2A-2C illustrate that macrophages infiltrate, cluster, and repress CD47-depleted syngeneic tumors in vivo only in combination with therapeutic opsonization by IgG. FIG. 2A Macrophage clusters in tumoroids are consistent with cooperative phagocytosis. (i) The informational entropy approximated by image file compression analysis (FIG. 10) summarizes macrophage clustering within CD47 KO tumoroids across multiple experimental conditions including anti-Tyrp1 opsonization, kinetic studies, macrophage priming with interferon-γ (IFNγ) or interleukin-4 (IL-4), and myosin-II inhibition with blebbistatin, as well as CD47 antibody blockade on WT tumoroids. Corresponding GFP+ tumoroid areas (normalized to areas on day 1) are shown for the same conditions (mean±SD, n=6-8 tumoroids). Statistical significance of entropy and tumor area differences were assessed by Welch's t-test (two-tailed, unpaired) for comparisons between two experimental conditions and one-way ANOVA with Tukey's multiple comparison test for the macrophage-priming experiment with three conditions. The representative thresholded macrophage images (top left) and fluorescence images of B16 and macrophage (bottom left) are from CD47 KO tumoroids ˜24 h after addition of 3:1 macrophage:B16 without anti-Tyrp1 (yellow frames) and with anti-Tyrp1 (green frames), which correspond to the first two bars in the graph. Scale bar: 100 μm. (ii) Representative max intensity projections of confocal fluorescence images (top) of CD47 KO tumoroids one day after addition of macrophages with and without anti-Tyrp1. Scale bar: 100 μm. Radial profiles (bottom) of macrophage and B16 fluorescence within CD47 KO tumoroids indicate macrophage clustering occurs only when tumoroids are opsonized with anti-Typr1 (mean±SD, n=3). FIG. 2B (i) Integrin activation downstream of Fc-receptor signaling when the CD47-SIRPα checkpoint is disrupted enhances phagocytosis and potentially increases macrophage adhesion in tumoroids. (ii) Representative images of opsonized, CD47 KO tumoroids ˜24 h after addition of macrophages and anti-Tyrp1 and 3 h later after addition of chelating agents (2 mM EDTA or EGTA) with or without 5 mM Mg2+. Scale bar: 100 μm. The macrophage cluster projected area increases significantly with Mg2+ and Ca2+ chelation by EDTA but not with Ca2+ chelation by EGTA (with or without additional Mg2+). Statistical significance was assessed by one-way ANOVA and Dunnett's multiple comparisons test between the PBS control and other conditions (mean±SD, n=7-8 tumoroids per group, representative of two experiments). FIG. 2C (i) Representative photographs of untreated and anti-Tyrp1-treated tumors prior to disaggregation and analysis by flow cytometry (top). Scale bar: 1 mm. Tumor area as measured on untreated or anti-Tyrp1-treated mice on day 5 of tumor growth, 24 h after treatment (bottom). Statistical significance was assessed by unpaired t-test (mean±SEM, n=3 per group). (ii) Representative fluorescence images of F4/80 staining in untreated or anti-Tyrp1-treated CD47 KO tumors on day 5, 24 h after treatment. White arrows denote nuclei within dashed white border of a macrophage engulfing multiple B16 cells (green dashed lines:B16 nuclei, orange dashed line: macrophage nuclei) while F4/80 (magenta) signal suggests macrophage clustering. Scale bar (4×): 0.5 mm, scale bar (40×): 50 μm, scale bar (40× zoom): 25 μm. (iii) CD45 and F4/80 staining of isolated tumor cells 24 h after a single dose of anti-Tyrp1 on day 4 after tumor engraftment. Anti-Tyrp1-treated tumors showed increased infiltrate of CD45+ cells as a fraction of total nucleated cells relative to untreated control, of which a ˜2-fold larger fraction of CD45+ cells were also F4/80+. Statistical significance was assessed by unpaired t-test with Welch's correction (n=3 per group for CD45+ staining) and by unpaired t-test (n=3 per group for F4/80+ staining), mean±SEM. (iv) Relationship between tumor area and F4/80+ macrophage immune infiltrate, fit with an exponential curve. F4/80+ cell frequency correlates negatively with tumor area on day 5. (v) Radial profile analysis of F4/80+ signal in CD47 KO tumors based on 4× images (n=3 per group). F4/80+ cells infiltrate more highly into the core of anti-Tyrp1-treated tumors and more highly overall, while control tumors show evenly distributed, lower overall F4/80+ infiltrate.

FIGS. 3A-3E illustrate that increasing macrophages boosts complete anti-tumor response rates of CD47-depleted tumors; engineered macrophages phagocytose cancer cells efficiently and clear WT tumors in a density-dependent fashion FIG. 3A (i) Representative tumor growth curves of projected tumor area versus days after tumor engraftment. Each symbol represents a separate tumor and is fit with an exponential growth equation A=A0^{ekt} (solid line). Complete anti-tumor responses in which a tumor was never palpable are all depicted with the same symbol (filled circle) and solid lines at A=0. For KO+ anti-Tyrp1, n=15 across 2 independent experiments are shown; for B16 ctrl+anti-Tyrp1, n=5 for one representative experiment. (ii) Survival curves of mice challenged with 2×105 WT or CD47 KO cells subcutaneously (s.c.) and treated i.v. with 250 μg anti-Tyrp1 on days 4, 5, 7, 9, 11, 13, and 15 or left untreated. Anti-Tyrp1 treatment has no effect on WT tumor survival (treated n=5, untreated n=9 across 1 and 2 independent experiments, respectively), but can eliminate ˜40% of CD47 KO tumors (treated n=82 across 10 independent experiments, untreated n=60 across 12 independent experiments). Long-term survivor mice were then used in subsequent rechallenge experiments in FIGS. 4A, 4B, and 4D. Donor bone marrow injections combined with i.v. anti-Tyrp1 treatment (n=11 across 2 independent experiments) increase the fraction of mice that show complete anti-tumor responses against CD47 KO tumors by 2-fold. Statistical significance was determined by the Log-rank (Mantel-Cox) test. The inset bar graph depicts response rates for CD47 KO tumors treated with anti-Tyrp1. A partial response was considered to be survival greater than one week beyond the median of the untreated CD47 KO cohort (16 d+7 d=23 d) and less than 80 d. FIG. 3B Scheme of two engineered macrophage anti-tumor therapeutic approaches. (top) Antibody-primed (with anti-Tyrp1, A′), Plus (P) SIRPα-blocked (by anti-SIRPα, B) marrow cells (A′PB, left). Fresh marrow is harvested from C57 donor mice, incubated with anti-tumor antibodies to prime FcRs and with anti-SIRPα to block CD47-SIRPα signaling. A′PB cells are then injected i.v. with additional antibody injections as indicated on days 4, 5, 7, 9, 11, 13, and 15 (bottom). Conditionally immortalized macrophage (CIM) progenitors derived from marrow cells Cas9 knock-in transgenic were also used to generate SIRPα KO macrophage progenitors as an improvement of antibody-based blockade. SIRPα KO CIM progenitor cells were then injected intravenously into tumor-bearing mice on day 4 (right) with indicated antibody i.v. injections on days 4, 5, 7, 9, 11, 13, and 15. FIG. 3C (i) Phagocytosis of WT B16 cells by bone marrow-derived macrophages. Macrophages with CD47-SIRPα signaling disrupted (through anti-CD47 or anti-SIRPα (A′PB)) and target IgG opsonization effectively engulf cancer cells in vitro. Statistical significance was assessed by one-way ANOVA with Sidak's test for multiple comparisons (mean±SEM, n=3 per condition). (ii) Survival curves of WT tumor-bearing mice (non-KO guide ctrl B16) treated i.v. with anti-CD47 and anti-Tyrp1 combined with either a one (1×) or two (2×) doses of A′PB (2×107 marrow cells i.v. per dose). The second A′PB dose was administered on day 7. Statistical significance was determined by the Log-rank (Mantel-Cox) test (untreated n=22 across 4 independent experiments, anti-Tyrp1/anti-CD47 n=6 in 1 experiment, anti-Tyrp1/anti-CD47/1×A′PB n=6 in 1 experiment, anti-Tyrp1/anti-CD47/2× A′PB n=8 in 1 experiment). FIG. 3D (i) Phagocytosis of WT B16 cells by CIMs. Unedited WT CIMs (left) engulf opsonized target B16 cells similar to bone marrow-derived macrophages with CD47-SIRPα disruption, while SIRPα KO CIMs (right) also engulf target B16 cells highly with opsonization. Statistical significance was assessed by one-way ANOVA with Sidak's test for multiple comparisons (mean±SEM, n=3 per condition). (ii) Survival curves of WT tumor-bearing mice (parental B16) treated i.v. with anti-Tyrp1 combined with a single dose of SIRPα KO CIM progenitors (4×106 cells i.v.) on day 4 with indicated antibody i.v. injections on days 4, 5, 7, 9, 11, 13, and 15. Significance was determined by the Log-rank (Mantel-Cox) test (n=5 per group). FIG. 3E Summary of complete anti-tumor response data showing that when CD47 is disrupted to maximize macrophage engulfment, WT B16 tumors can be eradicated in a macrophage density-dependent fashion. High macrophage transfer counts and permanent ablation of CD47-SIRPα signaling in either engineered macrophage or in tumors results in the highest rate of tumor elimination.

FIGS. 4A-4F illustrate that acquired anti-tumor immunity is required for long-term survival and protects against tumor re-challenge, experimental metastases, and target antigen loss. FIG. 4A Re-challenge and treatment scheme for long-term survivors from FIG. 3A-ii (top). Survival curves (bottom) of mice challenged s.c. with 2×105 CD47 KO B16 cells and treated with 250 μg anti-Tyrp1 i.v. on days 4, 5, 7, 9, 11, 13, and 15 after engraftment or left untreated. Mice challenged with CD47 KO a 2nd time and treated with the anti-Tyrp1 (n=17 across 4 independent experiments) survive at a rate ˜2.5-fold higher than treated age-matched naïve mice (n=12 across 3 independent experiments); tumors in untreated age-matched naïve mice (n=18 across 5 independent experiments) progress as expected. Long-term survivors challenged a 3rd time can reject tumors without further treatment (n=12 re-challenged and age-matched naive across 3 independent experiments). Statistical significance was determined by the Log-rank (Mantel-Cox) test. FIG. 4B Survival curves of mice bearing experimentally induced CD47 KO B16 lung metastases. Anti-Tyrp1 delays survival but fails to eliminate these metastases in mice (n=11 across 2 independent experiments) unless mice had previously eliminated subcutaneous KO tumors (n=11 across 2 independent experiments). Untreated lung metastasis-bearing mice, n=5. Statistical significance was determined by the Log-rank (Mantel-Cox) test. FIG. 4C Treatment scheme (top) and survival curves (bottom) of NSG mice engrafted with CD47 KO tumors and treated with anti-Tyrp1 i.v. (n=5) on days 4, 5, 7, 9 11, 13, and 15 or left untreated (n=5). Anti-Tyrp1 treatment modestly delayed tumor progression but failed to induce complete anti-tumor responses. Statistical significance was assessed by the Log-rank (Mantel-Cox) test. FIG. 4D Treatment scheme (top) and survival curves (bottom) of NSG mice engrafted with WT B16 tumors (non-KO guide ctrl) and treated with 4×106 SIRPα KO CIMs and anti-Tyrp1 i.v. (n=5 per group) on days 4 (or beginning day 2 when treatment began prior to day 4), 5, 7, 9 11, 13, and 15 or left untreated (n=5). Adoptive transfer of SIRPα KO CIMs at day 2 (n=5) and day 4 modestly delayed tumor progression but failed to induce complete anti-tumor responses. Statistical significance was assessed by the Log-rank (Mantel-Cox) test. FIG. 4E (i) Flow histograms of Tyrp1, CD47 KO cells (double knockout, DKO) showing complete depletion of the monoclonal antibody therapeutically targeted B16 antigen. (ii) Survival curves of DKO tumor-bearing mice that had previously survived CD47 KO B16 challenge (+anti-Tyrp1). 50% of mice survive (n=4) without additional treatment, while naïve mice challenged with CD47 KO tumors fail to survive (n=3). Statistical significance was determined by the Log-rank (Mantel-Cox) test. FIG. 4F Anti-B16 IgG emerges after tumor challenge and subsequent re-challenge. Western blotting of CD47 KO B16 lysate with serum as primary probe followed by anti-mouse IgG [H+L] secondary staining shows binding to proteins beyond Tyrp1 that increases in the 2nd tumor challenge.

FIGS. 5A-5D illustrate that convalescent serum IgG drives B16-specific phagocytosis and clustering in tumoroids and suppresses tumor initiation in vivo. FIG. 5A (i) Experimental scheme of phagocytosis assay with serum-opsonized DKO B16 cells or wild-type YUMM2. 1s. Convalescent serum collected from mice during and after tumor challenge contains polyclonal anti-B16 IgG that binds and opsonizes cells. (ii) Phagocytosis of serum-opsonized CD47 KO B16 cells, DKO B16 cells, or wild-type YUMM2. 1s. Convalescent serum from complete responder mice opsonizes B16 cells and drives phagocytosis even when Tyrp1 is deleted but does not do so against syngeneic YUMM2.1 cells that share a C57 antigen repertoire. Statistical significance was assessed by one-way ANOVA with Šídák's test for multiple comparisons (mean±SEM, opsonization conditions: n=9 convalescent sera, n=3 naïve sera, n=6 B16 with anti-Tyrp1, n=3 wells YUMM2.1 with anti-Tyrp1, n=6 unopsonized). FIG. 5B Kinetic profiles of anti-B16 IgG levels in convalescent sera across the three tumor challenges or in naive sera. Binding of IgG2a/c and IgG2b to CD47 KO (filled symbols) or DKO (open symbols) was measured with subclass-specific secondary antibodies and is reported as the median fluorescence intensity (mean±SEM, n=4 survivors, n=3 non-survivors, n=5 naive). FIG. 5C (i) Schematic of CD47 KO B16 pre-opsonization with serum or anti-Tyrp1 just prior to s.c. flank injection. (ii) Tumor growth curves at early timepoints where growth is still in the linear regime. Linear fits show significant growth suppression of CD47 KO B16 cells pre-opsonized with either anti-Tyrp1, convalescent serum, isotype IgG2a, or lacking opsonization (ctrl opsonization n=9, serum or anti-Tyrp1 opsonization n=5 per group). Statistical significance at each timepoint between ctrl (black) and combined opsonization (orange) was assessed by two-tailed unpaired t-test with Welch's correction after significant F test to compare variances. FIG. 5D (i) Convalescent sera repress KO tumoroid growth and drive macrophage clustering. Convalescent serum or anti-Tyrp1 can eliminate tumoroids within days whereas naïve serum or isotype control Ab do not. Solid lines are nonlinear regression of the data to a simple exponential of the form A(t)=A1 ekeff(t−1) (mean±SEM, n=3 or 4 tumoroids per sample for 9 convalescent and 9 naïve sera). (ii) Entropy measured by image file compression (FIG. 2A) correlates with the effective growth rate (Pearson r=0.71, p=0.0003). For simplicity, data are shown for convalescent sera that produced high (circles), medium (squares), and low (diamonds) entropy clusters (and low, medium, and high growth repression, respectively) and for a representative naïve serum sample (n=3 or 4 tumoroids per serum or antibody condition). Representative fluorescence images depict high and low entropy macrophages, respectively, in tumoroids on day 2, ˜24 h after addition of macrophages and naïve (top) or convalescent serum (bottom). Scale bar: 100 μm.

FIG. 6 is a diagram illustrating that cooperative macrophages eliminate tumor cells to initiate an acquired immune response that includes phagocytic feedback. Macrophage density and cooperativity, IgG-opsonization, and efficient blockade of the macrophage checkpoint underlie successful macrophage immunotherapy of solid tumors. Phase separating macrophages cooperatively engulf cancer cells when CD47 signaling is disrupted deeply and cancer cells are opsonized with IgG to maximize engulfment. Macrophage phagocytosis in tumors treated with anti-tumor IgG monoclonal antibody (mAb) initiates an acquired immune response required for long-term survival, which includes the production of de novo endogenous anti-tumor IgG that are capable of further opsonization for phagocytic feedback.

FIGS. 7A-7F illustrate the characterization of B16 tumoroids and subcutaneous tumors. FIG. 7A Brightfield and confocal microscopy of B16 tumoroids. (left) Brightfield image of a B16 tumoroid three days after 1000 cells were seeded atop an agarose gel. Large tumoroids typically have irregular borders and appear dark due to melanin pigments. Scale bar: 0.5 mm. (right) Confocal max intensity projections (xy, xz planes) of a B16 tumoroid GFP (green) and DNA stain (blue). The tumoroid was fixed ˜24 h after 100 cells were seeded in the non-adhesive well. Scale bar: 100 μm. FIG. 7B Tumoroid cohesion depends on Ca2+. (i) Transcriptomic analysis of cultured B16 cells (GSE162105) indicates N-cadherin and cadherin-19 are possible Ca2+-dependent cell-cell adhesion molecules. (ii) Immunoblotting of B16 lysate fractions confirms N-cadherin protein expression in the membrane fraction and expected lamin-A/C and tubulin distribution in the nuclear and cytoplasmic fractions, respectively. (iii) Treatment of B16 tumoroids with 2 mM EDTA or 2 mM EGTA results in a rapid (<1 h) increase in tumoroid projected area versus untreated tumoroids (n=14-16 tumoroids, Tukey box plot). Statistical significance was assessed by ordinary one-way ANOVA and Tukey's multiple comparisons test (p<0.0001 for EDTA vs. PBS and EGTA vs. PBS at t=1 and 3 h). Scale bar: 200 μm. FIG. 7C Tumoroid cohesion depends on the actin cytoskeleton. Treatment of B16 tumoroids with 1 μM Latrunculin A results in a rapid (<1 h) increase in tumoroid projected area versus treatment with DMSO vehicle (n=16 tumoroids, Tukey box plot). Statistical significance was assessed by Welch's t-test (unpaired, two-tailed). Scale bar: 200 μm. FIG. 7D Photographs of subcutaneous B16 tumors in mice dissected on day 4, 8, or 12 after subcutaneous (s.c.) tumor injection. Day 4 tumors were typically elongated compared to more spherical day 8 and day 12 tumors. Elongation was often observed in the direction in which the needle was inserted during subcutaneous injection of the cell suspension (left). Tumor area calculated in ImageJ is reported beneath each photograph. Scale bars: 10 mm (left) and 5 mm (right). FIG. 7E Trichrome-stained B16 tumor section. The tumor interior (region 1, inset scale bar: 50 μm) is cellular (maroon-stained cytoplasm and blacked-stained nuclei) with some necrosis. The tumor periphery contains blue-stained ECM fibers (region 2, inset scale bar: 100 μm) and large, round fat droplets. Scale bar: 1 mm. FIG. 7F Ex vivo pipette aspiration rheology of subcutaneous B16 tumors. (i) Cellular interior regions of tumors remain cohesive during aspiration like tumoroids and exhibit large extension L into the pipette at low ΔP (top left). Peripheral regions of tumors (fibrotic capsule and adjacent subcutaneous tissue) typically exhibit small L and require higher ΔP for measurable deformations (top right). Images depict the extension of tumor into the pipette after 90 s of aspiration at the indicated ΔP. Scale bar: 50 μm. (ii) Tissue strain L(t)/RP, where L(t) is the extension of the tissue into the pipette as a function of time and RP is the radius of the pipette, is plotted for the constant pressure creep phase (0-90 s) and after the pressure was released back to ΔP=0 for a representative interior region of a tumor. The profile is typical of a viscoelastic solid and the deformation was recoverable when the pressure was released. The black line depicts nonlinear regression of L(t)/RP to the standard linear solid model, which includes the elastic modulus E as a fitted parameter. (iii) Elastic moduli fit from the plateau in the strain vs. time plot for interior and peripheral regions of tumors excised days 4, 8, and 12 after engraftment (15-30 measurements per interior or periphery of each tumor, 2-3 tumors per time point, Tukey box plot). The solid lines are fits of simple exponential growth (periphery, E˜0.8et/10) or exponential decay (interior, E˜0.5e-t/10+0.3) where nonlinear regression of the data was constrained so rates and y-intercepts would be shared for the periphery and interior. The shaded box denotes the range of E reported for B16 cells 2, 3 that also includes typical E for B16 tumoroids.

FIGS. 8A-8G illustrate an analysis of CD47 and Tyrp1 expression and phagocytosis of B16 tumoroids and cell suspensions. FIG. 8A Flow cytometry analysis of CD47 and Tyrp1 expression on B16 cell lines. (i) Representative histograms show anti-CD47 (left) and anti-Tyrp1 (right) binding to CD47 KO (top) and WT (bottom) B16 cell lines, which were detected with secondary antibodies conjugated with Alexa Fluor 647. The populations shown in the histograms were gated on live, single cells using forward/side scatter and DAPI staining. (ii) Median fluorescence intensity calculated for CD47 KO and WT cells incubated with varying concentrations of anti-CD47 or anti-Tyrp1 followed by secondary antibody (n=3 tests per antibody concentration, mean±SD) were fit to a hyperbolic binding model of the form y=A*x/(K+x), which is shown as a solid line. The shaded regions correspond to blocking and opsonizing antibody concentrations that are required for tumoroid elimination in the FIG. 1-F. B Tyrp1 protein expression on B16 melanoma cells. (i) Immunofluorescence image of a B16 cell surface-stained (fixed, unpermeabilized) with anti-Tyrp1 and anti-mouse IgG Alexa Fluor 647 (green) and counterstained with Hoechst 33342 (blue). (ii) Brightfield image of a B16 cell showing pigmented melanosomes. Scale bars: 25 μm (left) and 10 μm (right). C Protein expression data from The Human Protein Atlas4 for CD47 (left) and Tyrp1 (right) across different normal tissues. CD47 is expressed in all tissues consistent with its role as a ‘marker of self’. Tyrp1 expression is limited to skin, and specifically to melanocytes. FIG. 8D Representative flow cytometry analysis of disaggregated tumoroids to measure phagocytosis. (i) Flow cytometry events were gated on live, single cells using forward/side scatter and Zombie aqua viability dye. Macrophages were gated based on CellTracker Deep Red fluorescence. (ii) The % phagocytic macrophages reported in FIG. 1C is calculated as the number of DeepRed+GFP+ double positive events divided by all DeepRed+ events multiplied by 100. FIG. 8E (i) Conventional 2D phagocytosis of WT and CD47 KO B16 cell lines by primary mouse bone marrow-derived macrophages (BMDMs). (ii) B16 cells (green) were incubated with opsonizing anti-Tyrp1 or mouse IgG2a isotype control antibody and added to adherent BMDMs (magenta). Yellow arrows denote phagocytic events, with some BMDMs engulfing multiple KO cells. Scale bar: 25 μm. (iii) The phagocytic index is calculated as the percentage of macrophages engulfing a target cell multiplied by the number of engulfed cells per macrophage (mean±SD, n=3 wells). Statistical significance was assessed by two-way ANOVA and Tukey's multiple comparison test. FIG. 8F (i) Tumoroid engulfment events consisting of GFP signal without co-localizing DNA signal (yellow arrows) were considered to be trogocytosis rather than phagocytosis as reported in FIG. 1D-i. Scale bar: 10 μm. (ii) Some B16 cells in tumoroids appear large and highly swollen (arrow), which may represent necrotic cell death that contributes to growth repression. Scale bar: 100 μm. FIG. 8G Phagocytic macrophages in tumoroids co-localize with melanin pigments, which is most easily visualized as a dark shadow in the GFP channel. The signal intensity in this region is below background light intensity, consistent with light absorbance by pigments. Scale bar: 100 μm.

FIGS. 9A-9F illustrate modeling cooperative tumoroid phagocytosis and non-cooperative phagocytosis in the conventional 2D assay. FIG. 9A Tumoroid growth is unaffected by anti-Tyrp1 added on day 1 without macrophages, indicating that anti-Tyrp1 has no direct cytotoxicity and that macrophages are required to mediate its effects (mean±SD, n=6 tumoroids). FIG. 9B (i) Macrophages do not significantly repress or eliminate CD47 KO or WT tumoroids in the absence of anti-Tyrp1 at any macrophage:B16 ratio tested (mean±SEM, n=6-8 tumoroids in a representative experiment). (ii) The effective growth rate is greater than 0 and shows little dependence on the macrophage:B16 ratio for unopsonized tumoroids (mean±SEM, n≥24 tumoroids across the four independent experiments shown in FIG. 1E). FIG. 9C Comparison of mathematical models for phagocytosis of tumoroids. (i) Workflow for fitting k_{eff} from tumoroid area (A) growth or decay curves (FIG. 1E) and subsequent model fitting of k_{eff} vs. macrophage number (M). Four models (models A-D) were considered including (model A) a Hill-like sigmoidal dependence on M, (model B) linear dependence on M, (model C) a power law dependence on M, and (model D) a sigmoidal dependence on the number of M.

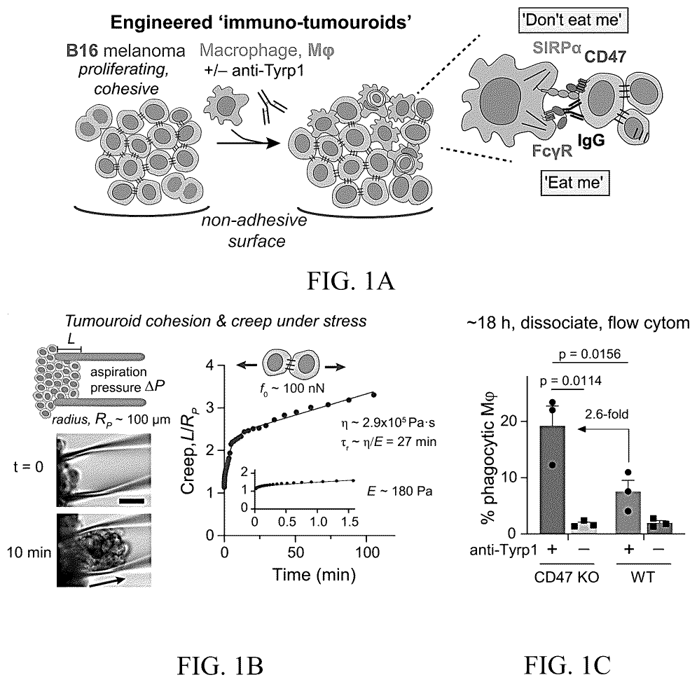

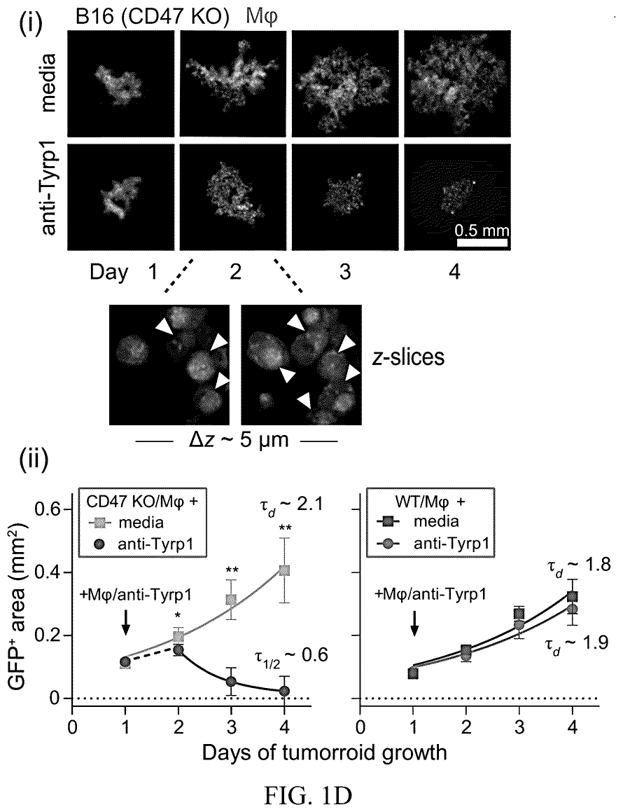

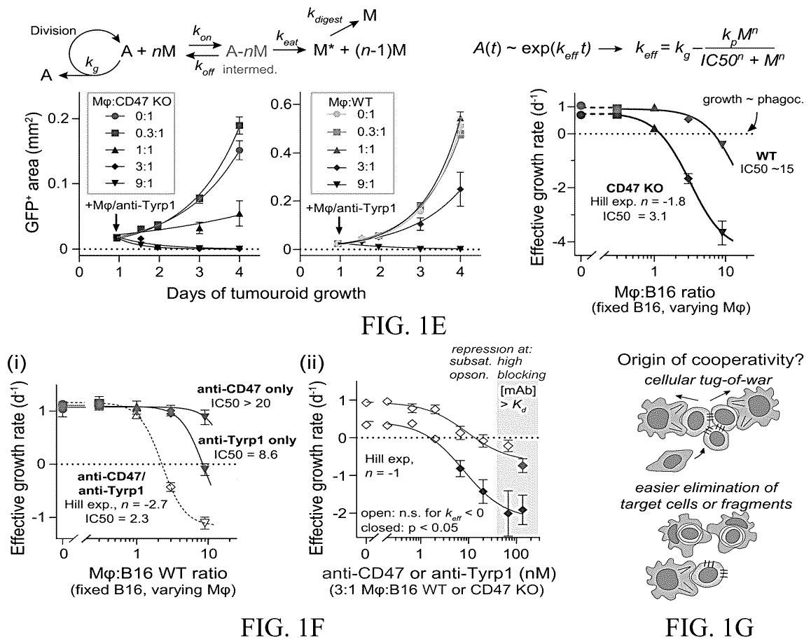

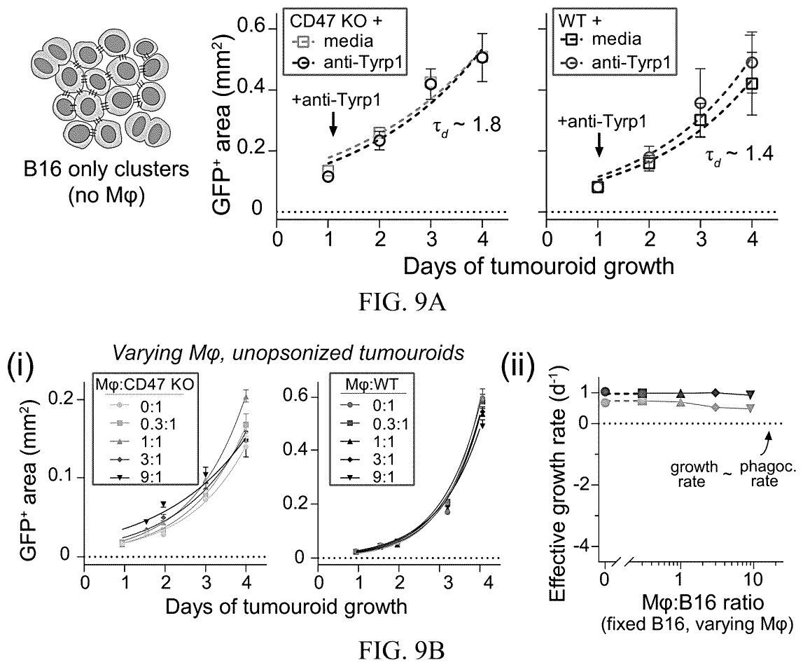

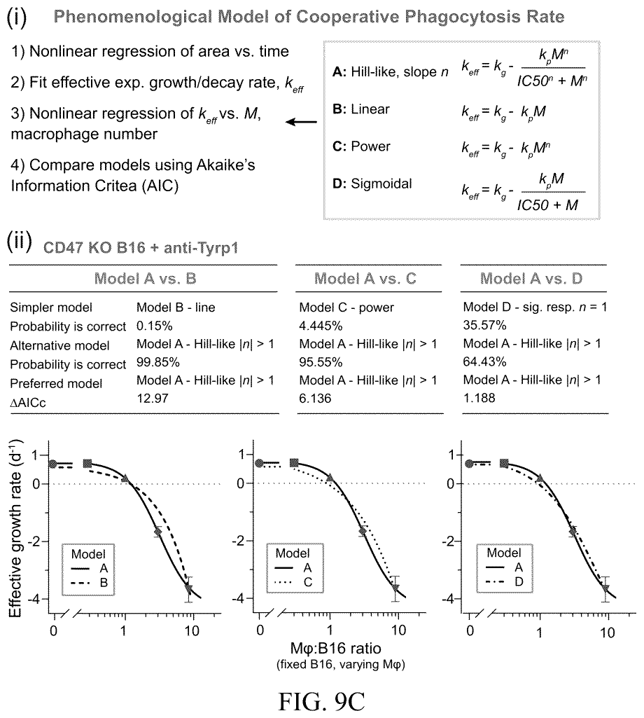

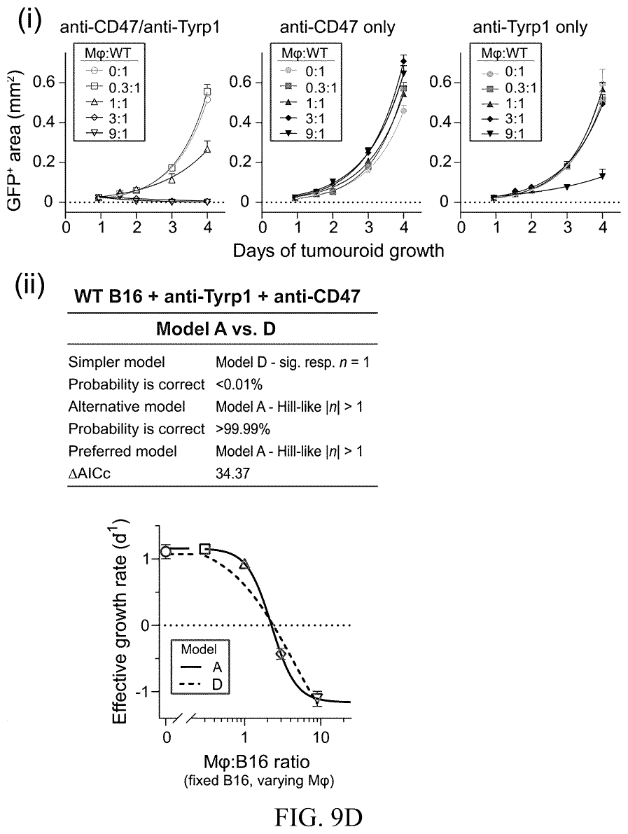

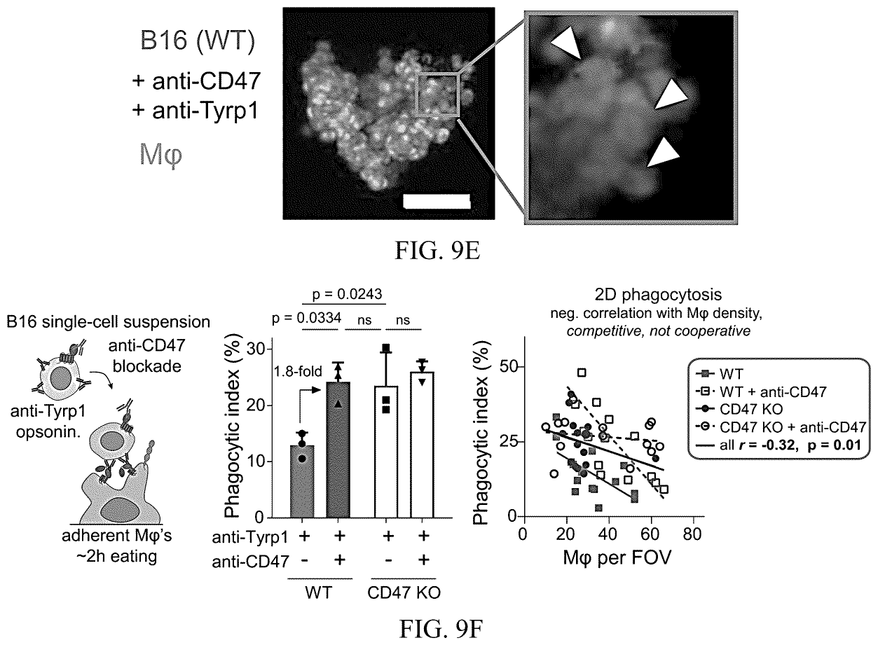

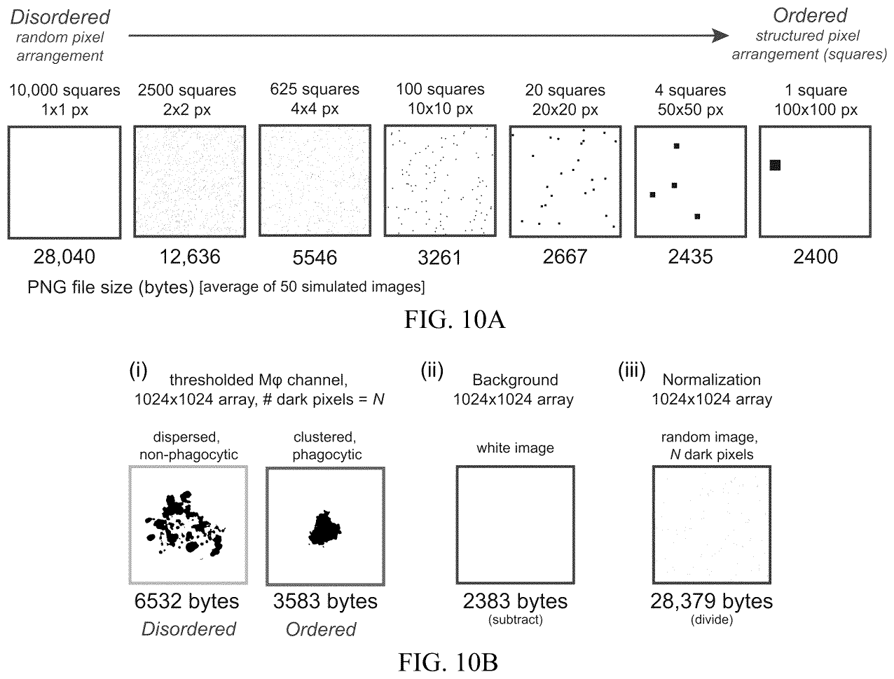

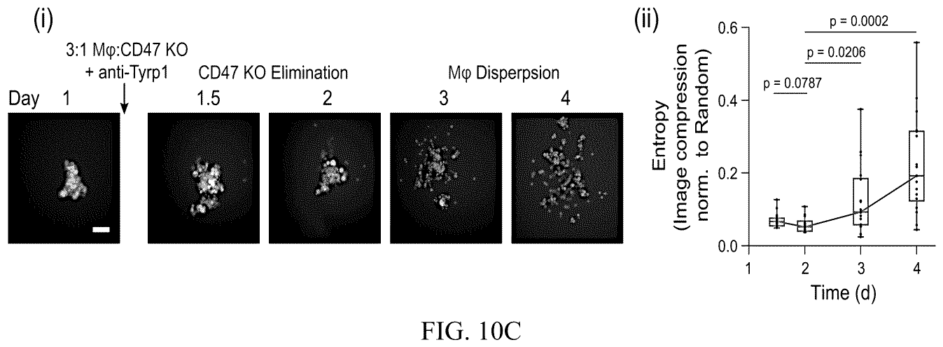

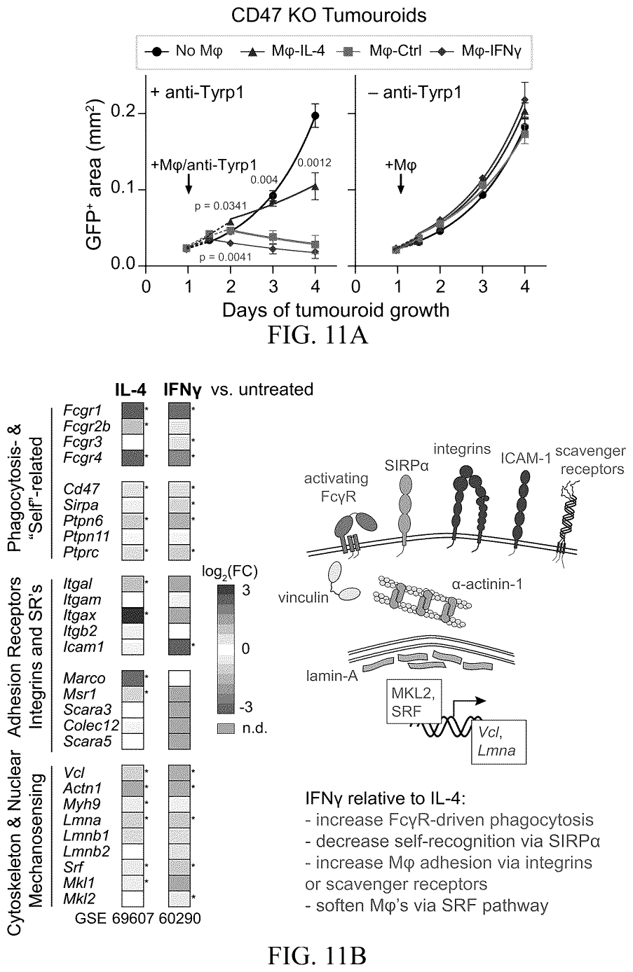

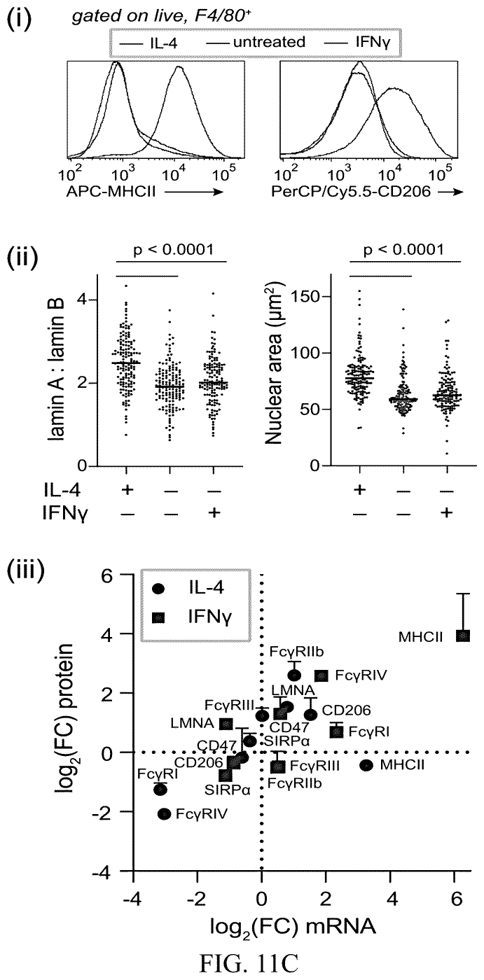

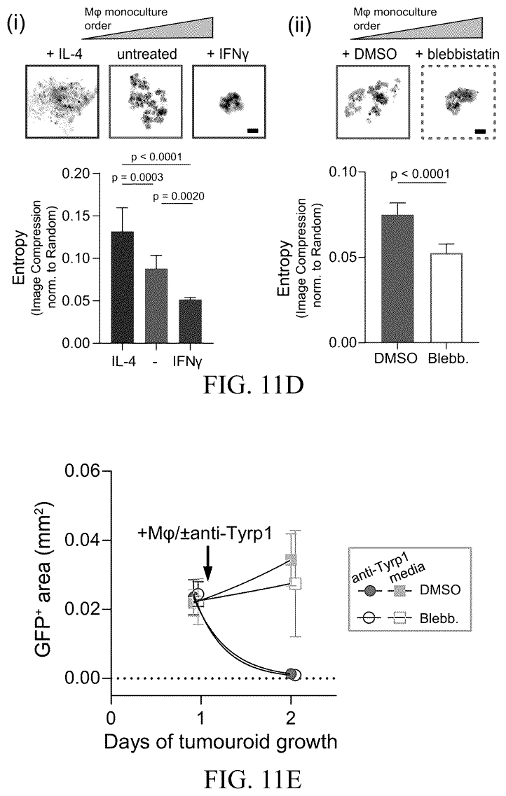

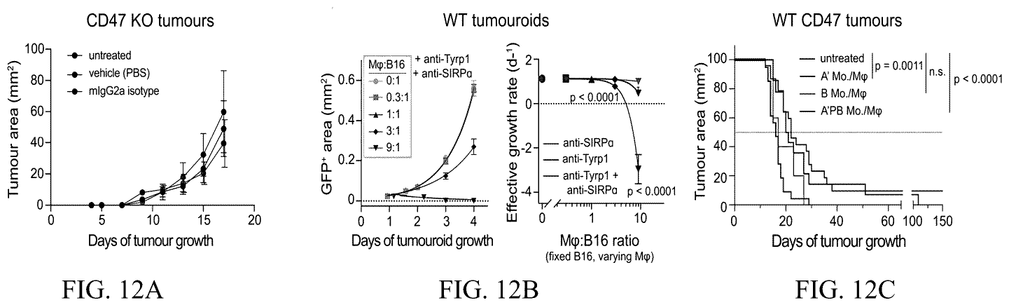

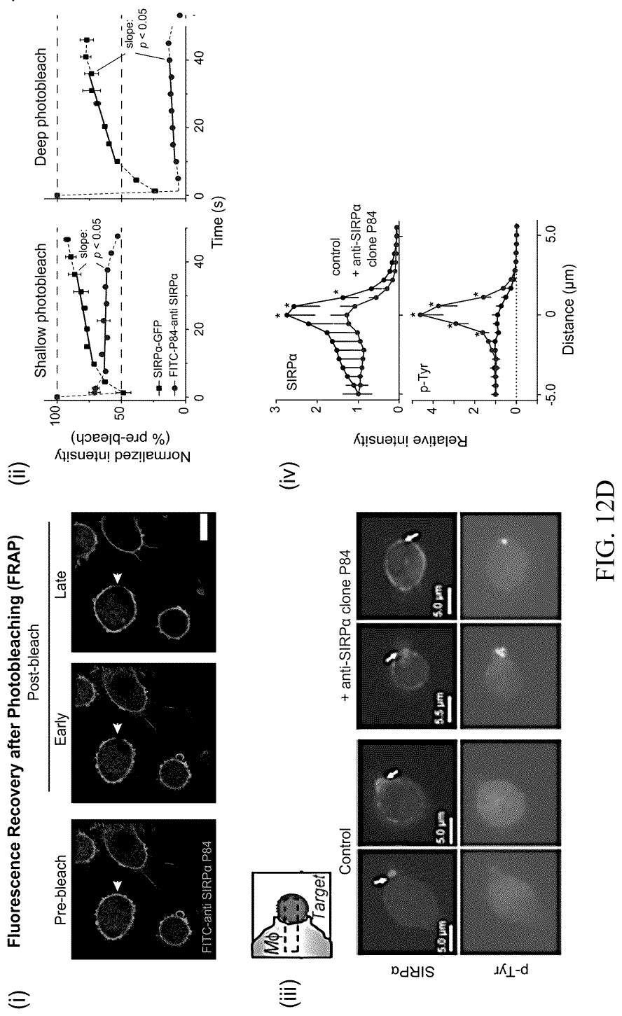

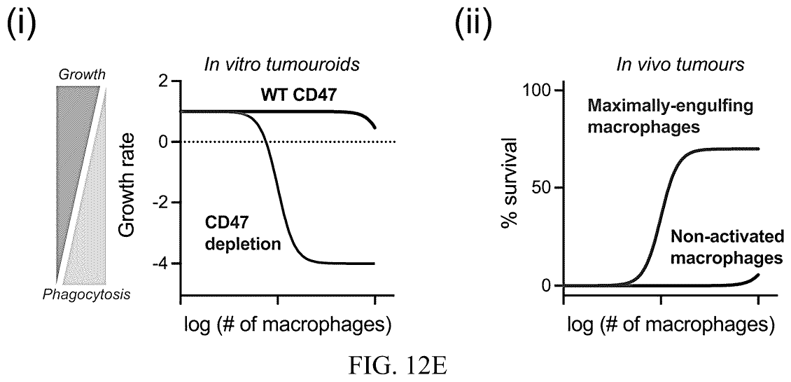

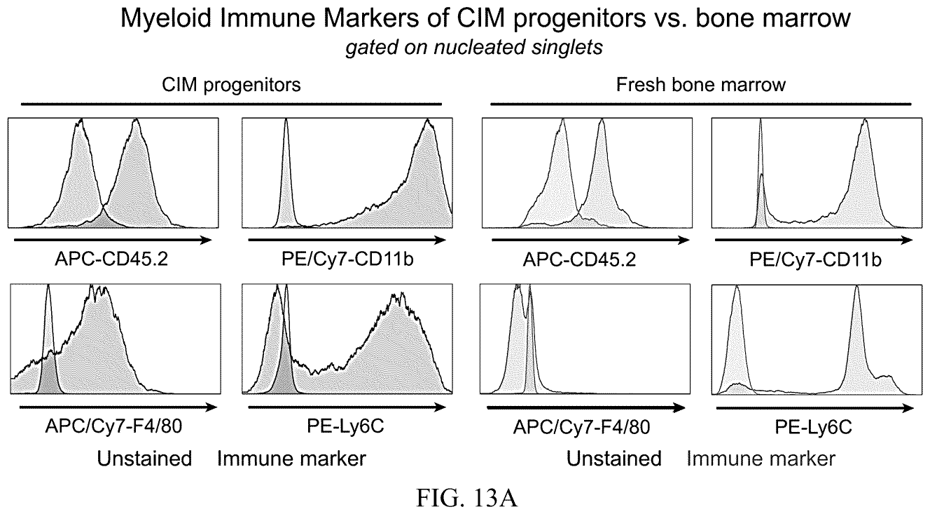

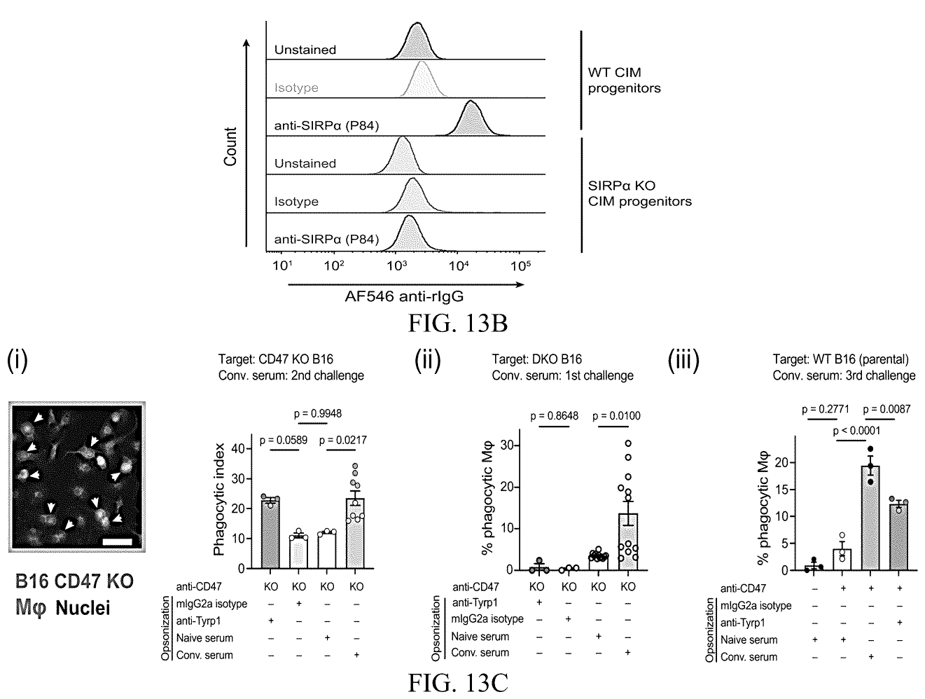

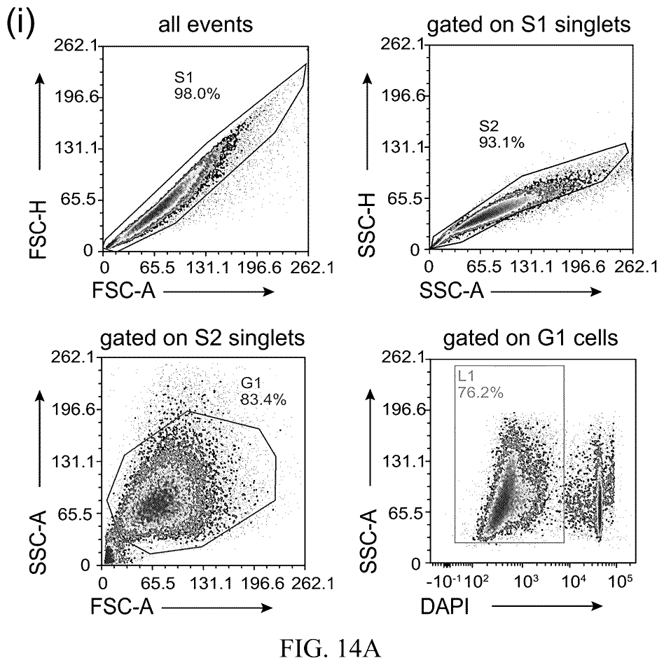

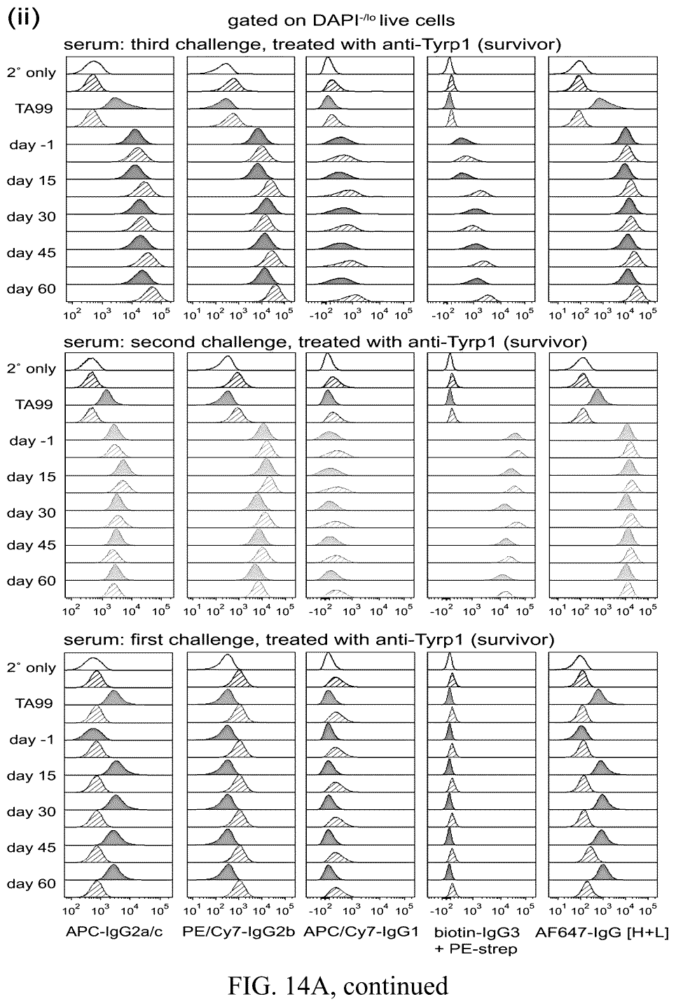

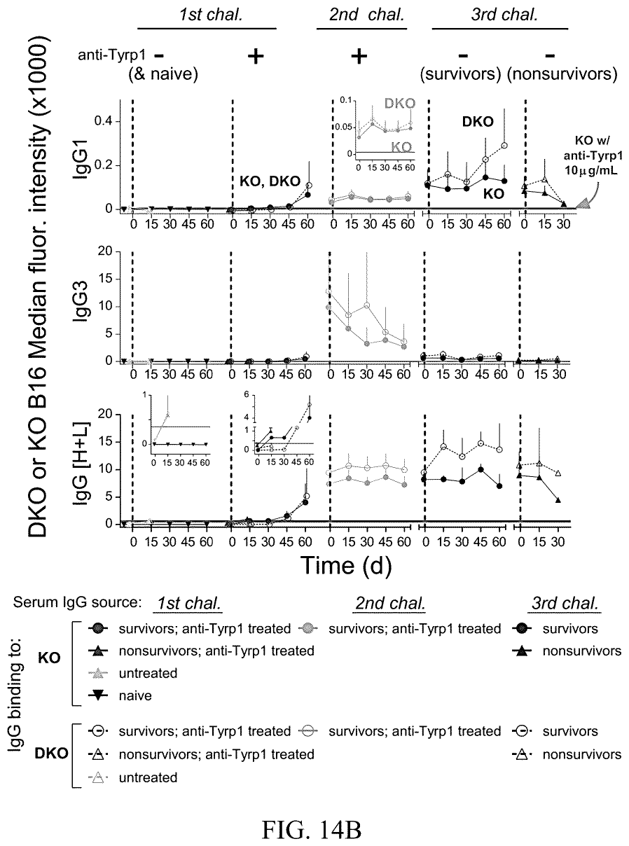

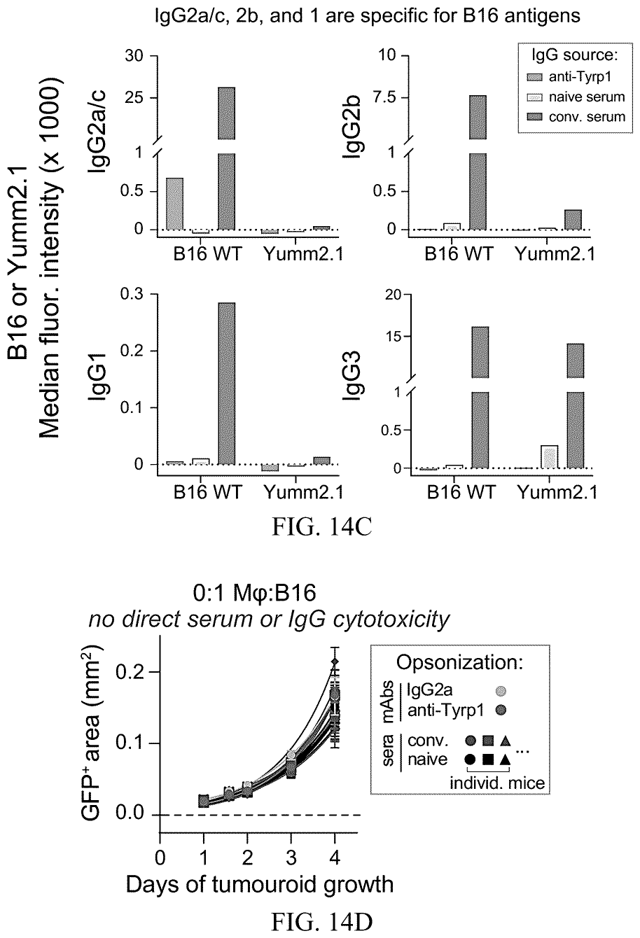

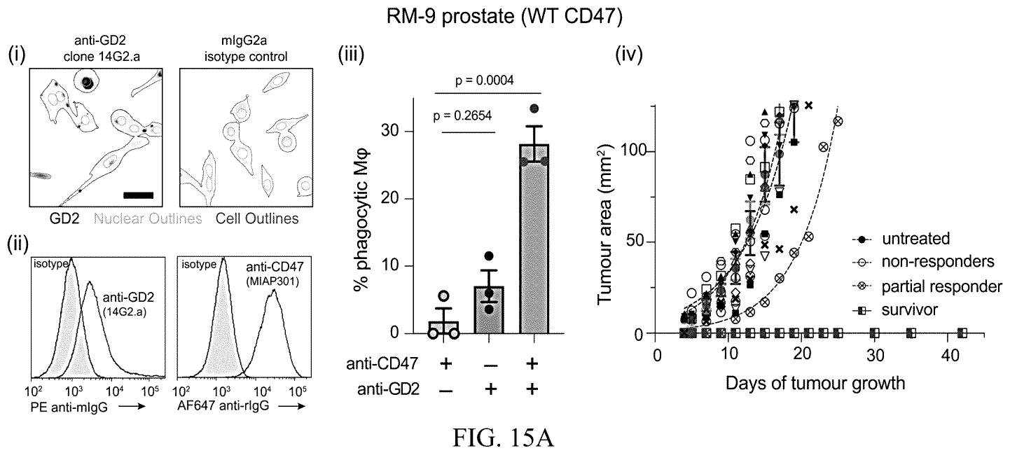

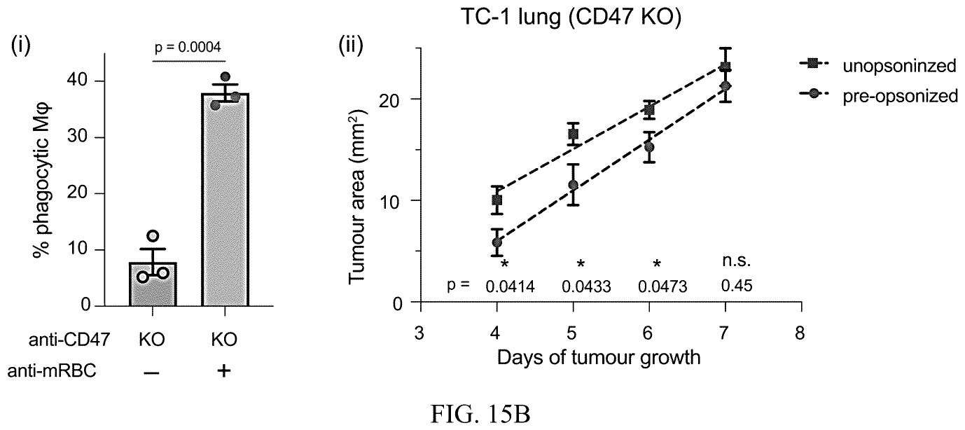

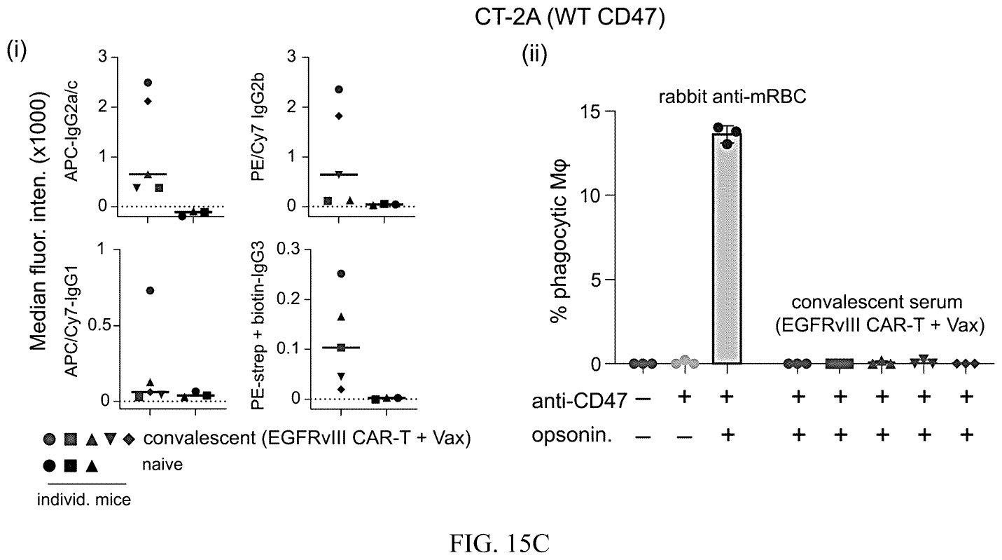

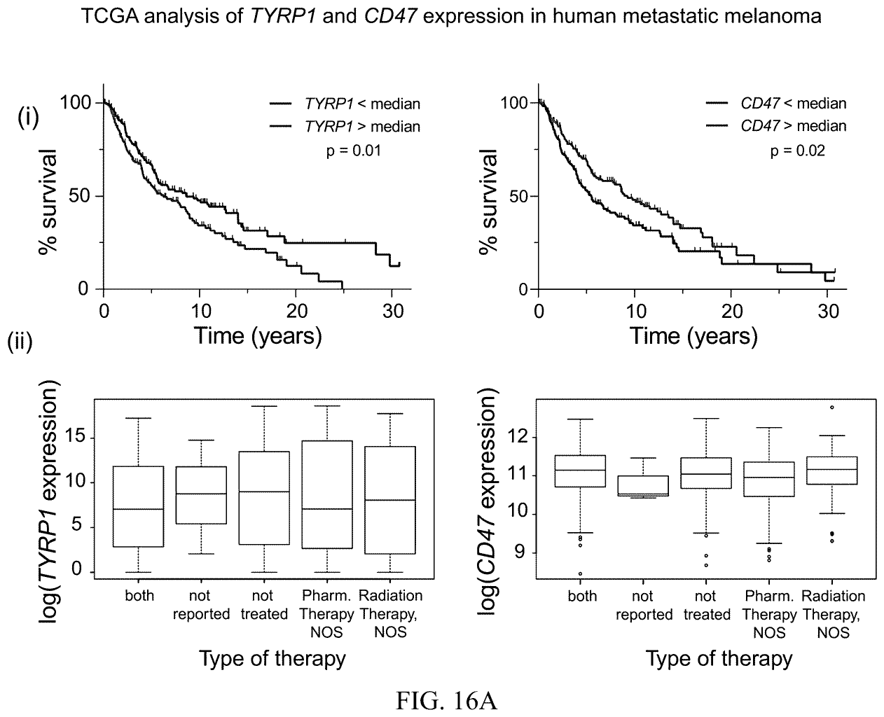

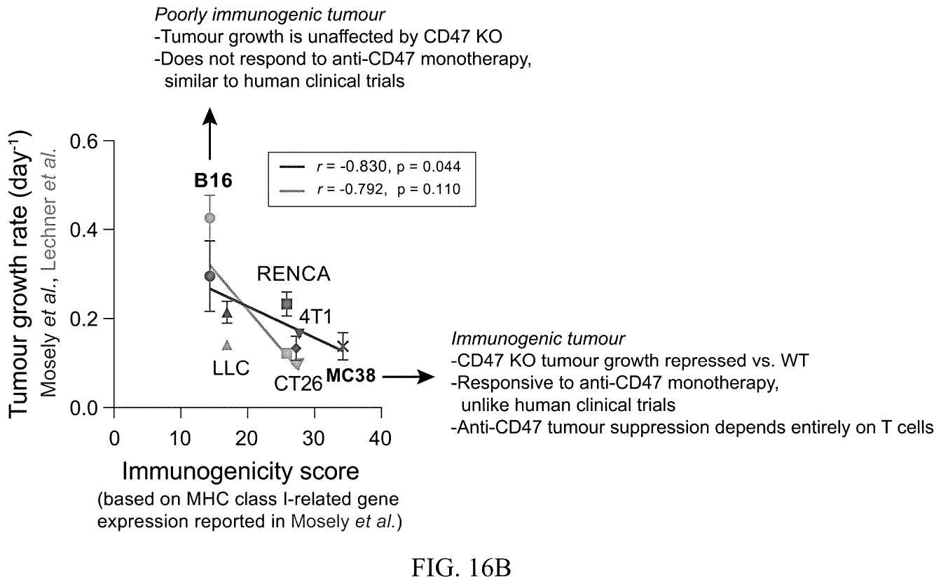

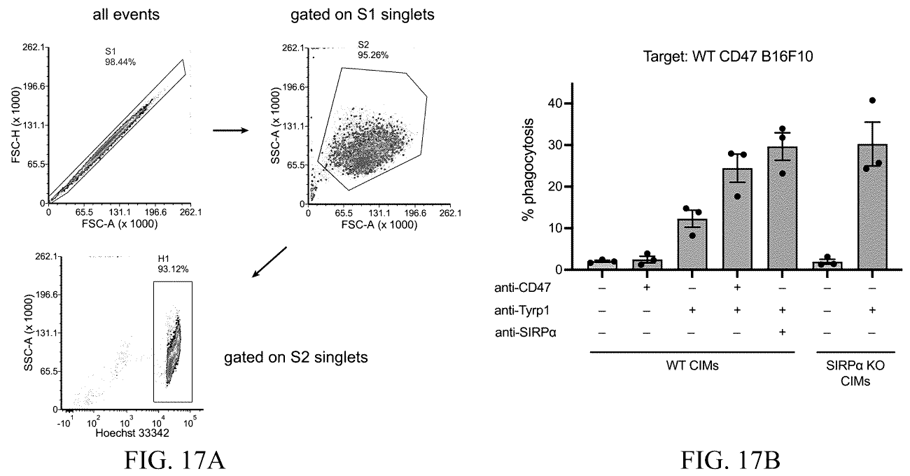

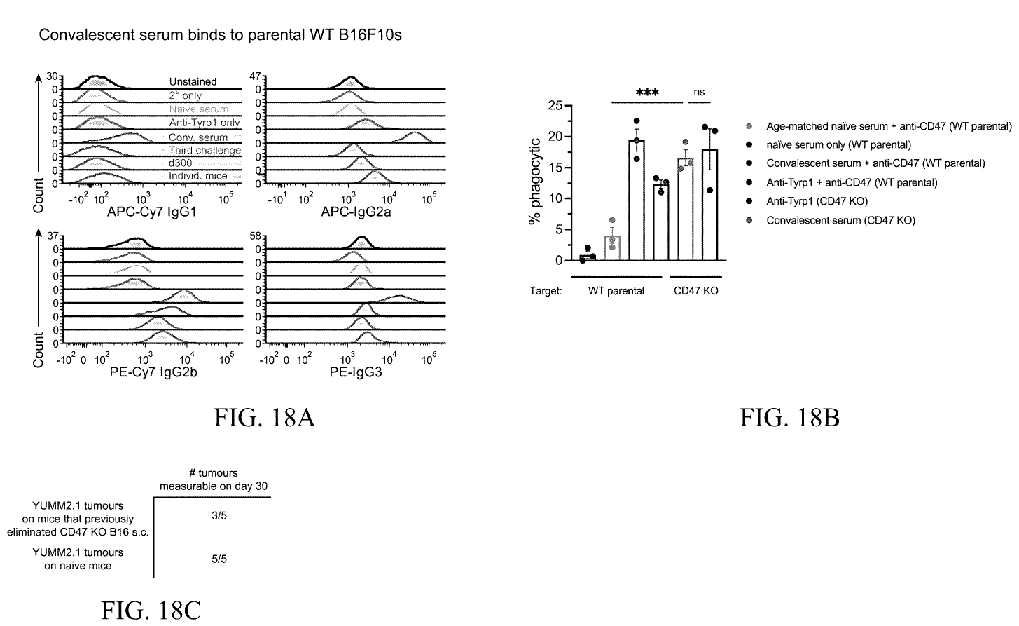

(ii) The fits of models B-D were compared one-by-one to model A using Akaike's Information Criteria (AIC) to determine whether the four parameter Hill-like model A is justified rather than comparatively simpler models with two or three parameters. The comparisons are summarized in the tables and the model fits to k_{eff} vs. macrophage:B16 ratio data (FIG. 1E) are plotted beneath. D (i) Growth curves for WT tumoroids treated with anti-CD47 and/or anti-Tyrp1 that correspond to the plot of k_{eff} vs macrophage:B16 ratio in FIG. 1F-i. (ii) The fits of models A and D were compared using Akaike's Information Criteria (AIC). The comparison is summarized in the tables and the fits are plotted beneath. FIG. 9E Representative max intensity projection of confocal images of a WT tumoroid one day after addition of macrophages, anti-Tyrp1, and anti-CD47. Scale bar 100 μm. The expansion depicts multiple macrophages that have engulfed B16 (denoted by white arrowheads). FIG. 9F Conventional 2D phagocytosis assay with adherent macrophages and single-cell suspensions of WT or CD47 KO B16 cells opsonized with anti-Tyrp1 and treated with either anti-CD47 or rat IgG2a isotype control antibody (mean±SD, n=3 wells per condition). Statistical significance was assessed by two-way ANOVA and Tukey's multiple comparison test. Across all four conditions (2 cell lines×2 blocking conditions), the number of macrophages per field of view (FOV) is plotted against the phagocytic index calculated for that FOV, revealing a weak negative correlation (Pearson's r=−0.32, p=0.01).

FIGS. 10A-10C illustrate an informational entropy calculation of macrophage order based on image compression. FIG. 10A Simulated 1024×1024 pixel (px) images containing 10,000 dark pixels on a white background that are arranged in random squares varying in size from 10,000 1×1 px squares to one 100×100 px square. The images were compressed in the portable network graphics (PNG) file format. The PNG file size (in bytes) below each image is the average of 50 randomly generated images with the same specifications. File size decreases with increasing image order/entropy. FIG. 10B To compare images with different numbers of dark pixels (N), a normalization procedure involved subtracting the PNG file size of a completely white image of the same dimension and then dividing this value by the PNG file size of an equal dimension random image with N dark pixels. FIG. 10C Macrophages disperse spontaneously following phagocytosis of tumoroids.

FIGS. 11A-11E illustrate the effects of cytokine priming or the myosin-II inhibitor blebbistatin on tumoroid growth suppression and macrophage clustering. FIG. 11A Tumoroid growth or repression with (left) or without (right) anti-Tyrp1 following addition of macrophages primed with either 20 ng/mL IFNγ or 20 ng/ml IL-4 for 48 h (mean±SEM, n=7-8 tumoroids). Statistical significance for each time point was assessed by one-way ANOVA and Dunnett's multiple comparison test between unprimed and IFNγ-primed or IL-4-primed macrophages. FIG. 11B Transcriptomic microarray analyses of BMDMs treated with IFNγ (GSE60290)5 or IL-4 (GSE69607)6 versus untreated BMDMs. The heat map depicts log 2 (fold change) for selected genes. Proteins encoded by differentially expressed genes are depicted in the schematic detailing a putative cytoskeleton and nuclear mechanosensing pathway and membrane receptors involved in phagocytosis and macrophage adhesion. Statistical significance was assessed by computing an adjusted p-value by the method of Benjamini and Hochberg (* p<0.05). FIG. 11C Measurements of protein expression of selected differentially expressed genes in BMDMs treated with IFNγ or IL-4 for 48 h. (i) Surface expression of MHCII and CD2016 in IFNγ- or IL-4-treated or untreated BMDMs was measured by flow cytometry. Similar staining was performed with antibodies against FcR's, CD47, and SIRPα. (ii) The ratio of lamin-A to lamin-B and the projected nuclear area were quantified by immunofluorescence microscopy (n>140 cells per condition across 8 fields of view). Statistical significance was assessed by one-way ANOVA followed by Tukey's multiple comparison test. (iii) The fold change (cytokine-primed vs. untreated) of indicated proteins from flow cytometry (i) (mean+SD, n=3 samples stained from MHCII and 4 samples for all other proteins) and lamin-A immunofluorescence staining (ii) is plotted against the fold change in gene expression from microarrays (panel B). FIG. 11D Entropy analysis for (i) cytokine-primed and (ii) blebbistatin-treated macrophage monocultures on non-adhesive surfaces at a density of 1000 cells per well (mean±SD, n=8 wells). Statistical significance was assessed by one-way ANOVA followed by Tukey's multiple comparison test for cytokine priming and Welch's t-test (unpaired, two-tailed) for blebbistatin treatment. Cytokines (20 ng/mL) or blebbistatin (20 μM) were added after 6 h and images were acquired after 48 h. Scale bars: 100 μm. FIG. 11E Tumoroid growth repression by macrophages was not affected by addition of 20 μM blebbistatin (mean±SD, n=3-4 tumoroids).

FIGS. 12A-12E illustrate isotype IgG2a control or vehicle injections and Fc-receptor-primed, SIRPα-blocked (A′PB) marrow cell injections and biophysical crosslinking mechanism the anti-mouse SIRPα mAb P84. FIG. 12A Isotype IgG2a control or vehicle injections have no effect on CD47 KO tumor growth relative to untreated tumors. Growth curves of mice bearing CD47 KO B16 tumors (2×105 s.c. flank injection) and treated i.v. on days 4, 5, 7, 9, 11, 13, and 15 with 250 μg isotype IgG2a control (clone C1.18.4), 100 μl PBS vehicle, or left untreated. Clone C1.18.4 is the isotype control antibody for anti-Tyrp1 clone TA99. There are no significant growth differences (mean±SEM, n=6 mice per group). FIG. 12B A′PB macrophages in tumoroids can effectively repress growth at high macrophage:B16 (WT CD47-non-KO guide ctrl) ratios, while anti-SIRPα or anti-Tyrp1 only conditions fail to control tumoroids. FIG. 12C Survival curves of mice bearing WT B16 tumors (parental line) and treated with 2×105 A′PB, A′ (Fc-receptor primed only, no SIRPα block), or B (no Fc-receptor priming, only SIRPα block) marrow cells i.v. on day 4 after tumor engraftment relative to untreated mice. A′PB and A′ conditions received additional 250 μg anti-Tyrp1 i.v. on days 5, 7, 9, 11, 13, and 15. FIG. 12D Anti-mouse SIRPα antibody clone P84 binds to mouse macrophages and immobilizes SIRPα. (i) Representative fluorescence images and (ii) FRAP analysis SIRPα-GFP diffusion±anti-mSIRPα clone P84. Sides of cells were bleached (yellow arrow) and then imaged to quantify recovery, which is lacking with P84 (n=3). (iii) Representative images of J774A.1 mouse macrophages±anti-mSIRPα clone P84 incubated with opsonized mouse RBCs opsonized with anti-mRBC for 30 minutes. Cells were fixed and stained for SIRPα and phospho-tyrosine (p-Tyr). (iv) Quantification of SIRPα and phospho-Tyr accumulation at the phagocytic synapse of mouse macrophages±anti-mSIRPα clone P84. Fluorescence intensity was quantified in each phagocytic synapse and normalized to fluorescence from macrophages that were not undergoing phagocytosis (mean±SEM, n=7). Statistical significance of each imaging bin along the phagocytic synapse was assessed by Welch's t-test (unpaired, two-tailed, Holm-Šidák correction for multiple comparisons, * p<0.05). FIG. 12E Summary of macrophage number versus therapeutic outcome for tumoroids in vitro and tumors in vivo. (i) Growth rate is controlled by tumor cell proliferation and macrophage phagocytosis. (ii) CD47 signaling dominates pro-phagocytic signaling at low and high macrophage numbers, but CD47 depletion, IgG opsonization, and macrophage infiltration can eliminate tumors.

FIGS. 13A-13B illustrate conditionally immortalized macrophage (CIM) progenitor engineering and opsonization of various B16 lines with convalescent serum. FIG. 13A Conditionally immortalized macrophage (CIM) progenitors are myeloid committed lineage cells that can be differentiated to a phagocytic macrophage phenotype and genetically engineered, such as with knockout of SIRPα. (i) CIM progenitors that are sourced from bone marrow of the Rosa26-Cas9 knock-in mouse on the C57BL6/J background 7, 8 express myeloid-specific surface markers. Immune marker staining on freshly harvested bone marrow cells is shown for comparison with lower F4/80 expression due to less lineage commitment compared to CIM progenitors. (ii) Flow histograms show SIRPα-KO CIM progenitors are depleted of SIRPα relative to unedited wild-type CIM progenitors. FIG. 13B Convalescent serum IgG from CD47 KO tumor complete responders binds and opsonizes both CD47 KO and WT CD47 B16 cells. FIG. 13C (i) Representative fluorescence image (left) showing serum opsonized CD47 KO B16 cells engulfed by BMDMs. Convalescent sera from second challenge survivors (serum collected 70 days after tumor challenge) opsonizes CD47 KO B16 cells for phagocytosis by BMDMs as effectively or more highly than anti-Tyrp1 (right). Statistical significance was assessed by one-way ANOVA and Šídák's multiple comparison test between selected groups (n=3 wells for antibody conditions, n=3 wells per serum sample for one naïve serum and three convalescent sera, mean±SEM). (ii) Convalescent sera from first challenge survivors (serum collected 99 days after tumor challenge) opsonizes DKO B16 cells that lack Tyrp1. Statistical significance was assessed by one-way ANOVA and Šídák's multiple comparison test between selected groups (n=3 wells for antibody conditions, n=3 wells for convalescent or naïve sera×3 wells for each sample, mean±SEM). (iii) Convalescent serum from a third challenge survivor opsonizes WT B16 cells blocked with anti-CD47, indicating that de novo opsonizing IgG is not an artifact of antigens found only on engineered B16 lines. Statistical significance was assessed by one-way ANOVA and Šídák's multiple comparison test between selected groups, ns not significant (n=3 wells per condition, mean±SEM).

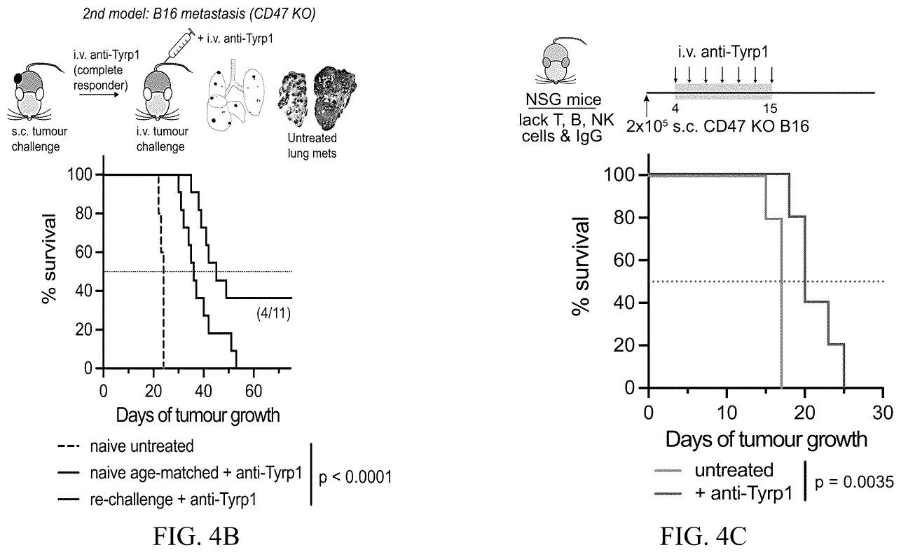

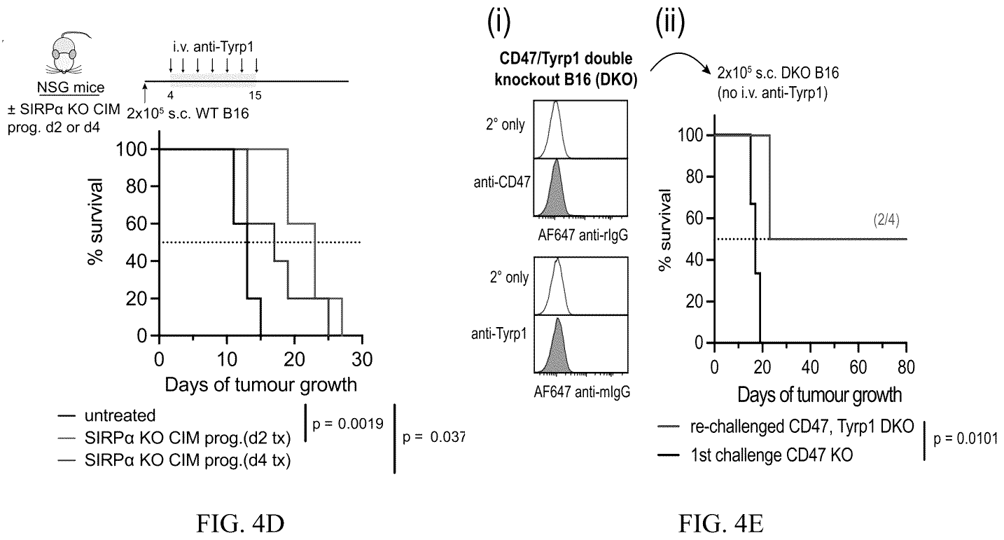

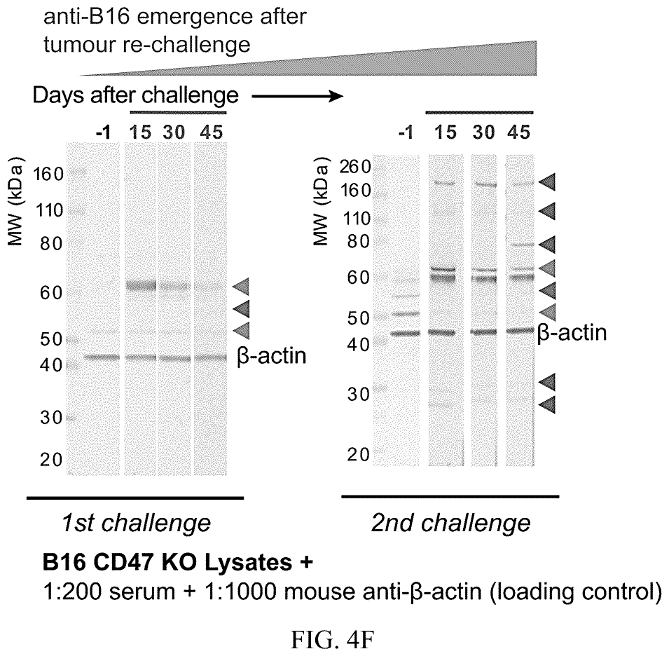

FIGS. 14A-14D illustrate that convalescent serum IgG from CD47 KO tumor complete responders shows subclass-specific binding to CD47 KO B16 cells and DKO B16 cells that lack Tyrp1, and no binding of IgG1, IgG2a/c, and IgG2b to non-B16 YUMM2.1 cells. FIG. 14A Flow cytometry analysis of serum IgG binding to B16 cell lines. (i) General gating strategy for flow cytometry analysis of IgG binding to B16 cell lines. Doublet discrimination was performed with pulse height and pulse area parameters in the forward and side scatter channels (S1 gate, FSC-H vs. FSC-A) and (S2 gate, SSC-H vs. SSC-A). Cells were distinguished from debris as shown in the G1 gate in the SSC-A vs. FSC-A plot). Dead cells were excluded from analyses by DAPI staining when the analysis was to be performed immediately on viable cells or by Zombie aqua dye when the analysis was to be performed at a later time after fixing cells. (ii) Representative flow cytometry histograms of KO (solid filled) and DKO (diagonal hashed) cells incubated with 5% (v/v) serum collected from mice undergoing KO tumor 1st, 2nd, or 3rd challenge on days −1, 15, 30, 45, and 60 after tumor inoculation. Cells were stained with a panel of monoclonal secondary antibodies specific for mouse IgG1, IgG2a/c, IgG2b, and IgG3 or with a polyclonal secondary antibody recognizing heavy and light chains [H+L] of all mouse IgG subclasses. Control samples were stained with either secondary antibodies only or 10 μg/mL anti-Tyrp1. Binding of anti-Tyrp1 was detected only with the IgG2a/c and polyclonal IgG [H+L] secondary antibodies. FIG. 14B Kinetics of de novo IgG generation revealed by flow cytometry analysis of KO (filled symbols) and DKO cells (open symbols) stained as described in A-ii with serum and with secondary antibodies specific for mouse IgG1 and IgG3 (rows 1-2) or a polyclonal anti-mouse IgG [H+L] secondary antibody (row 3). The corresponding data for IgG2a/c and IgG2b are shown in FIG. 4B. The median fluorescence intensity was corrected by subtracting the background fluorescence equal to the signal of cells incubated with secondary antibodies only. Symbols and error bars depict the mean±SEM of each group. The range of staining observed with 10 μg/mL anti-Tyrp1 is denoted by the pink boxes or lines. FIG. 14C Median fluorescence intensity of B16 WT or Yumm2.1 cells incubated with 5% (v/v) serum collected from a mouse that survived the third challenge, with naïve serum, or with anti-Tyrp1 followed by staining with a panel of monoclonal secondary antibodies specific for mouse IgG1, IgG2a/c, IgG2b, and IgG3. FIG. 14D Convalescent sera do not exhibit direct cytotoxic toward KO tumoroids and require macrophages to suppress growth and eliminate tumoroids (FIG. 4D). Tumoroids treated with convalescent serum, naïve serum, anti-Tyrp1, and IgG2a isotype control all grow similarly. Solid lines are nonlinear regression of the data to a simple exponential of the form A(t)=A1 ekeff(t−1) (mean±SEM, n=3 or 4 tumoroids per sample for 9 convalescent and 9 naïve sera).

FIGS. 15A-15C illustrate a generalization of CD47 disruption and macrophages in prostate, lung, and glioblastoma tumor models FIG. 15A In vitro and in vivo phagocytosis of WT RM-9 prostate tumors (i) Immunostaining shows cluster(s) of anti-GD2 on each cell detected by secondary antibody conjugated with Alexa Fluor 647. The clusters are consistent with ‘cap formation’ by antibodies that crosslink mobile epitopes linked together as should occur due to GD2 association with rafts or due to the polyclonal secondary antibody. No clusters are visible with control Ab. Scale bar: 100 μm. (ii) Flow cytometry histograms of anti-GD2 clone 14G2.a (left) and anti-CD47 clone MIAP301 (right) binding to RM-9. The bound primary antibodies were detected with secondary antibodies conjugated with PE and Alexa Fluor 647, respectively. The populations shown in the histograms were gated on live, single cells using forward/side scatter and DAPI staining. (iii) GD2 is targetable for opsonization and drives phagocytosis of RM-9 cells by BMDMs in 2D phagocytosis assays when combined with anti-CD47. Statistical significance was assessed by one-way ANOVA with Šidák's test for multiple comparisons (mean±SEM, n=3 per condition) (iv) Representative WT RM-9 tumor growth curves of projected tumor area versus days after tumor engraftment. Each symbol represents a separate tumor. Mice were treated with 4×106 SIRPα KO CIM progenitors i.v. on day 4 and 250 μg anti-GD2 i.v. on days 4, 5, 7, 9, 11, 13, and 15 or left untreated. Complete anti-tumor responses (“survivor”, 1) in which a tumor was never palpable are all depicted with the same symbol (half-filled square). Untreated control tumors (n=5) and non-responder tumors (n=8) are also shown. Exponential growth curves fit well to the mean growth behavior, and the variance of untreated and non-responders grow with time as expected—all of, which all adds high confidence to the measurements. FIG. 15B Pre-opsonization of TC-1 lung tumors delays early growth. (i) TC-1 tumors are targetable by anti-mouse RBC opsonization. CD47 KO and opsonization enables potent phagocytosis by BMDMs in 2D phagocytosis assays. Statistical significance was assessed by two-tailed unpaired t-test (mean±SEM, n=3 per condition) (ii) Tumor growth curves at early timepoints where growth is still in the linear regime. Linear fits show significant growth suppression of WT TC-1 cells pre-opsonized with anti-mouse RBC and anti-CD47 relative to no opsonization ctrl (ctrl opsonization n=10, pre-opsonized on=10). Statistical significance at each timepoint between ctrl (pink) and pre-opsonized (teal) was assessed by two-tailed unpaired t-test with Welch's correction after significant F test to compare variances, ns not significant. FIG. 15C Binding and functional analysis of convalescent serum IgG from vaccine-enhanced CAR-T therapy against CT-2A glioma. (i) Convalescent sera from long-term survivors contain IgG's of all four murine subclasses that bind to the surface on CT-2A cells. The reported data are median fluorescence intensity (corrected by subtracting background signal measured on unstained cells) from flow cytometry histograms of CT-2A cells stained with 5% v/v serum followed by the same secondary Ab panel used for FIG. 4 and FIGS. 14A-14D (median with individual points, n=5 convalescent and 3 naïve sera). (ii) CT-2A cells treated with convalescent sera as in (i) were not phagocytosed by BMDMs even when CD47 was blocked. As a positive control, CT-2A were opsonized with 5% v/v anti-mRBC polyclonal antiserum (mean±SD, n=3 wells per serum sample).

FIGS. 16A-16B illustrate the potential for combination immunotherapy in human melanoma and rationale for CD47 combination therapy versus monotherapy in syngeneic mouse models. FIG. 16A TCGA analysis of TYRP1 and CD47 in human melanoma. (i) Survival analysis of metastatic melanoma patients in The Cancer Genome Atlas (TCGA) based on expression of TYRP1 (left) and CD47 (right) above or below the median. Significance was determined by the Log-rank (Mantel-Cox) test. (ii) Expression levels of TYRP1 (left, p=0.68, one-way ANOVA) and CD47 (right, p=0.36) were not dependent on treatment reported in TCGA. FIG. 16B Meta-analysis of tumor growth from data reported by Mosely et al. 11 and Lechner et al. 12 shows that growth rate across the different syngeneic mouse tumor models is anti-correlated (Pearson r<0) with an immunogenicity score defined as the summation of log 2 (expression) of MHC class I-related genes reported by Mosely. Graphical growth data were digitized using WebPlotDigitizer version 4.2 and growth curves were fit to the exponential growth equation V(t)=V0ekt to determine the tumor growth rate k (±SE). Tumor types are melanoma (B16), lung (LLC), kidney (RENCA), breast (4T1), and colon (CT26 and MC38). Note that B16 doubling rates in the studies presented herein are consistent with the data here, even with CD47 knockout.

FIGS. 17A-17B illustrate that conditional immortalized macrophage (CIM) progenitors are myeloid committed lineage cells that can be differentiated to phagocytic macrophage phenotype. FIG. 17A illustrates the gating strategy for flow cytometry evaluation of CIM progenitors. FIG. 17B illustrates phagocytosis of wildtype CD47 B16F10 target cells by wildtype or SIRPα knockout CIMs receiving the indicated antibody treatments.

FIGS. 18A-18C illustrate that convalescent serum IgG from CD47 KO tumor complete responders binds and opsonizes both CD47 KO and WT CD47 B16s. FIG. 18A depicts flow histograms of 4 (CD47 KO) third challenged (serum drawn 300 days after tumor challenge) mice showing that IgG in convalescent serum from complete responder mice binds to WT B16s. Anti-B16 IgG2a and IgG2b subclasses bind equivalently to or more highly than anti-Tyrp1. FIG. 18B depicts flow histograms of four (CD47 KO) third challenged (serum drawn 300 days after tumor challenge) mice showing that IgG in convalescent serum from complete responder mice binds to WT B16s. Anti-B16 IgG2a and IgG2b subclasses bind equivalently to or more highly than anti-Tyrp1. FIG. 18C shows that mice that showed complete anti-tumor responses after three challenges of CD47 KO B16 tumors also grow YUMM2.1 tumors.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of +20% or +10%, more preferably +5%, even more preferably +1%, and still more preferably +0.1% from the specified value, as such variations are appropriate to perform the disclosed methods. Unless otherwise clear from context, all numerical values provided herein are modified by the term about.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein to “alleviate” or “treat” a disease, disorder or condition means reducing the severity of one or more symptoms of the disease, disorder or condition.

The terms “binding,” “bind,” “bound” refer to an interaction between two molecules. The interaction may include a covalent or non-covalent bond. The interaction may also be reversible or irreversible depending on the type of interaction, such as covalent bond formation.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, uterine cancer, and the like.

By “CD47 antagonist” or “SIRPα antagonist” is meant a molecule that does not provoke a biological response. The CD47 or SIRPα antagonist can prevent or decrease ligand- or agonist-mediated CD47 and or SIRPα responses. The CD47 or SIRPα antagonist may have affinity for CD47 or SIRPα and bind either receptor where binding disrupts the interaction between CD47 and SIRPα, thereby inhibiting CD47 or SIRPα activation. Other CD47 or SIRPα antagonists may have affinity for CD47 or SIRPα ligands or agonists and compete with CD47 or SIRPα receptors to bind the ligands or agonists, thereby preventing or reducing the ability of a ligand or agonist to bind the receptor. Typically, these CD47 or SIRPα antagonists have a greater affinity or a lower dissociation constant for the CD47 or SIRPα ligands or agonists than the receptor.

The term “CRISPR/CAS”, “clustered regularly interspaced short palindromic repeats system”, or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

The term “CRISPRi” refers to a CRISPR system for sequence specific gene repression or inhibition of gene expression, such as at the transcriptional level.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “effective amount” or “therapeutically effective amount” are used interchangeably herein, and refer to an amount of a compound, formulation, material, or composition, as described herein effective to achieve a particular biological result. Such results may include, but are not limited to, the reduction in tumor size as determined by any means suitable in the art.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

The phrases “an immunologically effective amount”, “an anti-immune response effective amount,” “an immune response-inhibiting effective amount”, or “therapeutic amount” refer to the amount of the composition of the present invention to be administered to a subject which amount is determined by a physician, optionally in consultation with a scientist, in consideration of individual differences in age, weight, immune response, type of disease/condition, and the health of the subject (patient) so that the desired result is obtained in the subject.

As used herein, an “instructional material” includes a publication, a recording, a diagram, or any other medium of expression that can be used to communicate the usefulness of the compositions and methods of the invention. The instructional material of the kit of the invention may, for example, be affixed to a container that contains the nucleic acid, peptide, and/or composition of the invention or be shipped together with a container that contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. “Purified” can also refer to a molecule separated after a bioconjugation technique from those molecules which were not efficiently conjugated.

By “macrophage” is meant a type of innate immune cell that phagocytose cellular material and act as an antigen presenting cell to other immune cells. Macrophages further stimulate and regulate inflammation by releasing cytokines.

By “monocyte” is meant a precursor to macrophage. Monocytes circulate in the bloodstream and differentiate into macrophages or dendritic cells when they infiltrate tissues. An “isolated nucleic acid” refers to a nucleic acid segment or fragment that has been separated from sequences that flank it in a naturally occurring state, e.g., a DNA fragment that has been removed from the sequences that are normally adjacent to the fragment, e.g., the sequences adjacent to the fragment in a is genome that it naturally occurs. The term also applies to nucleic acids that have been substantially purified from other components that naturally accompany the nucleic acid, e.g., RNA or DNA or proteins, that naturally accompany it in the cell. The term therefore includes, for example, a recombinant DNA that is incorporated into a vector, into an autonomously replicating plasmid or virus, or into the genomic DNA of a prokaryote or eukaryote, or that exists as a separate molecule (e.g., as a cDNA or a genomic or cDNA fragment produced by PCR or restriction enzyme digestion) independent of other sequences. It also includes a recombinant DNA that is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) that “U” replaces “T.”

As used herein, an “opsonin” is a molecule that binds to the surface of a particle (e.g., antigen) to enhance the process of phagocytosis. In one aspect, an opsonin can include an “opsonizing antibody”, which refers to an antibody that binds to the surface of a cell, coating the negatively charged molecules on the cell membrane, and enhancing the uptake of the cell by a phagocyte (e.g., macrophage). In another aspect, complement proteins or fragments thereof such as C3b and C4b can bind to cell surfaces and act as opsonins. The term “opsonization” refers to the process in which an opsonin binds to the surface of an antigen so that the antigen will be readily identified and engulfed by phagocytes for destruction. In certain embodiments, “opsonins” and “opsonizing antibodies” can include the targeting antibodies used to enhance the phagocytic activity of the modified phagocytic cells of the invention. In other embodiments, “opsonins” and “opsonizing antibodies” are different from the targeting antibodies.

The term “phagocyte” or “phagocytic cell” is used to refer to an immune cell that is capable of engulfing or ingesting harmful particles, bacteria, infected cells, dead or dying cells, and other cells targeted for phagocytosis. Phagocytic cells include, but are not limited to, macrophages, monocytes, mast cells, neutrophils, and dendritic cells. In one embodiment, the phagocyte is a macrophage or a monocyte. In another embodiment, the phagocyte is a bone marrow cell or monocyte progenitor cell that is capable of differentiating into a mature macrophage by culture in specific cytokines or chemokines (e.g., CSF and/or GM-CSF). Such differentiating processes are typically permanent, and as such are referred to as “terminal differentiation”.

The terms “phagocytosis” and “phagocytic activity” as used herein refer to the process of engulfing and ingesting particles or cells by an immune cell. The phagocytic cell engulfs or ingests the particle or cell by binding to the particle or cell coated with opsonins.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, that there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

A “constitutive” promoter is a nucleotide sequence that, when operably linked with a polynucleotide that encodes or specifies a gene product, causes the gene product to be produced in a cell under most or all physiological conditions of the cell.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

As used herein, the term “repressor” refers to a molecule or compound which eliminates, decreases, or suppresses the interaction of CD47 with SIRPα. The repressor can prevent or interfere with the interaction between the proteins by inhibiting expression or decreasing expression of one or both proteins, masking or hiding the binding site of one or both proteins, interfering with one or more functions of one or both proteins. Repressors can include, but are not limited to, anti-CD47 antibody, CD47-shRNA, CD47-siRNA, anti-SIRPα antibody, SIRPα-shRNA, SIRPα-siRNA, CD47 antagonists, SIRPα antagonists, anti-CD47-SIRPα antibody, a CRISPR system, and any combination thereof.

As used herein, the term “repressor of SIRPα” refers to a molecule or compound which eliminates, decreases, or suppresses the SIRPα expression, activity, and/or function. The repressor can prevent or interfere with SIRPα by inhibiting expression or decreasing expression of the protein, masking or hiding the binding site on SIRPα, and interfering with one or more functions of SIRPα. Repressors can include, but are not limited to, anti-SIRPα antibody, SIRPα-shRNA, SIRPα-siRNA, SIRPα antagonists, a CRISPR system, and any combination thereof.

As used herein, “sample” or “biological sample” refers to anything, which may contain the cells of interest (e.g., macrophages) for which the screening method or treatment is desired. The sample may be a biological sample, such as a biological fluid or a biological tissue. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

The terms “SIRPα,” “SIRPA,”, or “SIRPα” are meant to refer to signal regulatory protein-alpha (SIRPα). SIRPα, also tyrosine-protein phosphatase non-receptor type substrate 1 or CD172A, is a member of the SIRP family and belongs to the immunoglobulin superfamily. SIRPα is a receptor-type transmembrane glycoprotein known to be involved in the negative regulation of receptor tyrosine kinase-coupled signaling processes.

By “SIRPα antagonist” is meant a molecule that does not provoke a biological response. The SIRPα antagonist can prevent or decrease ligand- or agonist-mediated SIRPα responses. The SIRPα antagonist may have affinity for SIRPα and binds the receptor to disrupt the interaction between CD47 and SIRPα, thereby inhibiting SIRPα activation. Other SIRPα antagonists may have affinity for SIRPα ligands or agonists and compete SIRPα receptors to bind the ligands or agonists, thereby preventing or reducing the ability of a ligand or agonist to bind the receptor. Typically, these SIRPα antagonists have a greater affinity or a lower dissociation constant for the SIRPα ligands or agonists than the receptor.

As used herein, “siRNA” and “small interfering RNA” are used interchangeably and refer to small oligonucleotides of single or double-stranded (ds) RNA used in RNA interference (RNAi). The siRNA can have a length of about 5 to about 50 nucleotides long. The siRNA also may have 3′ overhangs at one or both ends. In one embodiment, the siRNA can be used to decrease or eliminate SIRPα gene expression.

As used herein, “shRNA” or “small hairpin RNA” or “short hairpin RNA” are used interchangeably and refer to an RNA molecule with a hairpin turn that can be used in RNA interference (RNAi). The shRNA can have a length of about 10 to about 100 nucleotides long. Expression of the shRNA in cells can be obtained by delivery of plasmids or viral or bacterial vectors. In one embodiment, the shRNA can be used to decrease or eliminate SIRPα gene expression.

By the term “specifically binds,” as used herein, is meant a compound, e.g., a protein, a nucleic acid, an antibody, and the like, which recognizes and binds a specific molecule, but does not substantially recognize or bind other molecules in a sample.

The term “subject” is intended to include living organisms that an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

The term “targeting antibody” as used herein refers to an antibody or antibody fragment that binds to an antigen on a target cell. The targeting antibody may recognize an antigen that acts as a cell surface marker on a target cell associated with a particular disease state, such as a viral, bacterial or parasitic infection, an autoimmune disease, or a cancerous cell state.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or improving a disorder and/or symptom associated therewith. It will be appreciated that, although not precluded, treating a disorder or condition does not require that the disorder, condition or symptoms associated therewith be completely ameliorated or eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION

The present invention provides phagocytic cells or precursor cells thereof that have been modified to express an estrogen receptor (ER)-Hoxb8 fusion protein that allows large numbers of cells to be rapidly expanded ex vivo. Withdrawing the ER ligand and culturing the cells in macrophage differentiating growth factors such as stem cell factor (SCF) and granulocyte-macrophage colony stimulating factor (GM-CSF) induces the expanded cells to differentiate into functional macrophage-like cells capable of phagocytic and cytotoxic function. The modified cells further comprise mutations or deletions to the endogenous genetic locus which encodes SIRPα such that expression of SIRPα protein is greatly reduced or lost. This modification eliminates the signaling interaction with CD47 on target cells and further enhances phagocytic function of the modified cells. The modified cells are also coated with targeting antibodies specific for one or more target cell antigens such that the Fc receptors on the surface of the modified cells are bound to and occupied by target cell-specific antibodies. Together these modifications provide highly phagocytic macrophage-like cells which have a specificity for any target cell which expresses the targeting antibody antigen. In certain embodiments, the modified phagocytic cells of the invention are specific for tumor-associated antigens or antigens expressed by tumor cells or tissues. As such, in certain aspects, the invention also provides methods of treating conditions or diseases, particularly cancers, comprising administering effective amounts of the modified phagocytic cells of the invention which are specific for antigens associated with the disease (e.g., tumor antigens).

Phagocytosis is the process in which a cell engulfs a solid particle, forming an internal vesicle called a phagosome. This process enables the immune system to remove pathogens and cell debris. Macrophages, a type of phagocytic cell, are derived from monocytes. Monocytes are recruited to sites of tissue damage or infection, then differentiate into macrophages and dendritic cells that help clear the pathogens and cell debris by phagocytosis. Macrophages discriminate between foreign (pathogens) and self-antigens via receptors on their cell surface.

In some aspects, this invention relates to disrupting the interaction between CD47 and protein signal regulatory protein-alpha (SIRPα) by genetically modifying phagocytic cells or precursor cells thereof. SIRPα, also known as tyrosine-protein phosphatase non-receptor type substrate 1 or CD172A, is a member of the SIRP family and belongs to the immunoglobulin superfamily. SIRP family members are receptor-type transmembrane glycoproteins known to be involved in the negative regulation of receptor tyrosine kinase-coupled signaling processes. CD47, an integrin-associated cell surface glycoprotein protein, has been demonstrated to be a ligand for SIRPα and, upon interaction with SIRPα on phagocytes, provides a “don't eat me” signal, thus acting as a marker of self-tissues and preventing phagocyte-mediated autoimmune tissue destruction.

SIRPα has also been shown to be an inhibitory phagocyte receptor, and its interaction with CD47 expressing erythrocytes is the main inhibitory signal of erythrophagocytosis. CD47 expressed on the surface of normal tissue cells prevents elimination of these cells by binding to the inhibitory receptor SIRP-alpha on the surface of phagocytes. Once activated, SIRPα inhibits pro-phagocytic signals from Fc and complement receptors, resulting in inhibition of phagocytosis. Thus, macrophages presenting SIRPα on their surfaces rely on interaction with CD47 to identify the particle as being self or foreign.

### Conditional Transformation of Monocyte Progenitors

In some aspects, the invention of the present disclosure provides modified phagocytic cells or precursor cells thereof which have been modified to be conditionally or reversibly transformed such that large numbers of cells can be quickly expanded ex vivo. In certain embodiments, the conditional transformation is accomplished by modifying the cells such that they express an estrogen receptor-Hoxb8 fusion protein. Also known as homeobox protein Hox-B8, Hoxb8 is a member of the Antp homeobox family of transcription factors. Under normal conditions, Hox family genes are associated with body pattern development and hematopoiesis. Hoxb8 in particular has been demonstrated to control the expansion of hematopoietic stem cells. Deregulation of Hox gene expression is associated with leukemias. Hoxb8 in particular, was the first Hox-family gene demonstrated to be an oncogene, where it cooperates with interleukin-3 (IL-3) signaling to drive the development of acute myeloid leukemia (AML).

In certain embodiments of the present invention, the Hoxb8 protein is fused to the ligand binding domain of the estrogen receptor. In the absence of cognate ligand, estrogen receptors are excluded from the cell nucleus, and upon ligand binding traverse the nuclear membrane to initiate changes to gene expression. In this way, the function of the ER-Hoxb8 fusion protein of the invention is dependent on presence of an ER ligand, such as β-estradiol. In certain embodiments, activation of the ER-Hoxb8 fusion protein maintains the undifferentiated, stem cell-like state of the phagocytic progenitor cells and allows for the expansion of large numbers of cells. Withdrawal of the ER ligand and culture of the cells in certain growth factors (e.g., CSF and GM-CSF) drives the terminal differentiation of the progenitor cells into macrophage-like cells capable of phagocytic function. In the absence of ER ligand, these fully differentiated and phagocytically functional cells are no longer immortalized. Such ER-Hoxb8 conditional transformation systems have been described previously, for example in WO2013158819 and Wang, et al. (2006) Nature Methods. April; 3(4):287-93.

### Modification of Signal Regulatory Protein-Alpha (SIRPα) Expression

In one aspect, the invention includes modified phagocytic cells, such as a macrophages or monocytes, and modified precursor cells thereof which comprise modified expression of signal regulatory protein-alpha (SIRPα) protein.

In certain embodiments, the modification can inhibit or decrease expression of SIRPα. Such modifications can be accomplished by the use of a SIRPα-shRNA, SIRPα-siRNA, a CRISPR system targeted to SIRPα, and a combination thereof.

In one embodiment, the modification comprises the expression of a SIRPα-shRNA. The shRNA includes RNA molecules with a hairpin turn that can be used in RNA interference (RNAi). The shRNA can have a length of about 10 to about 100 nucleotides long. Expression of the shRNA in cells can be obtained by delivery of plasmids or viral or bacterial vectors. In one embodiment, shRNA can be used to decrease or eliminate SIRPα gene expression.

In one embodiment, the modification comprises a SIRPα-siRNA. The siRNA includes small oligonucleotides of single or double-stranded (ds) RNA used in RNA interference (RNAi). The siRNA can have a length of about 5 to about 50 nucleotides long. The siRNA also may have 3′ overhangs at one or both ends. In one embodiment, the siRNA can be used to decrease or eliminate SIRPα gene expression.

In one embodiment, the modification is a CRISPR system. CRISPR is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR system can thereby be engineered to cleave virtually any DNA sequence by redesigning the gRNA in cell lines (such as 293T cells), primary cells, and CAR T cells. The CRISPR system can simultaneously target multiple genomic loci by co-expressing a single CAS9 protein with two or more gRNAs, making this system uniquely suited for multiple gene editing or synergistic activation of target genes.

One example of a CRISPR system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR system gene disruption occurs when a guide nucleic acid sequence specific for a target gene and a Cas endonuclease are introduced into a cell and form a complex that enables the Cas endonuclease to introduce a double strand break at the target gene. In one embodiment, the CRISPR system comprises an expression vector, such as, but not limited to, an pAd5F35-CRISPR vector. In one embodiment, a modified T cell is generated by introducing a Cas expression vector and a guide nucleic acid sequence specific for a gene into a T cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

In one embodiment, the CRISPR system comprises a Cas expression vector, such as an inducible promoter inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In another embodiment, the CRISPR system comprises a guide nucleic acid sequence specific for SIRPα. The guide nucleic acid sequence targets SIRPα for Cas endonuclease-induced double strand breaks. The sequence of the guide nucleic acid sequence may be within one or more loci of the SIRPα genes. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

In another embodiment, the CRISPR system comprises a guide nucleic acid sequence specific for CD47 and/or SIRPα. The guide nucleic acid sequence targets CD47 and/or SIRPα for Cas endonuclease-induced double strand breaks. The sequence of the guide nucleic acid sequence may be within one or more loci of the CD47 and/or SIRPα genes. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence may be specific for any gene, which encodes a gene product that interferes with CD47-SIRPα interaction, such as a gene product that would modulate expression of the SIRPα gene.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In another embodiment, the repressor is a TALEN system. TALENS are artificial restriction enzymes generated by fusing a TAL effector DNA binding domain to a DNA cleavage domain. TALENs uses a nonspecific DNA-cleaving nuclease fused to a DNA-binding domain that can be to target essentially any sequence. For TALEN technology, target sites are identified, and expression vectors are made. See Liu et al, 2012, J. Genet. Genomics 39:209-215. The linearized expression vectors (e.g., by Notl) is used as template for mRNA synthesis. See Joung & Sander, 2013, Nat Rev Mol Cell Bio 14:49-55.

TALENs and CRISPR methods provide one-to-one relationship to the target sites, i.e., one unit of the tandem repeat in the TALE domain recognizes one nucleotide in the target site, and the crRNA or gRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination.

### Targeting Antibody

In certain embodiments, the modified phagocytic cell or precursor cell thereof of the invention is also bound or coated by a targeting antibody. The targeting antibody may bind the phagocytic cell through a Fc receptor on the phagocytic cell, such as a macrophage, monocyte or bone marrow cell. In one embodiment, a modified macrophage is exposed to the targeting antibody simultaneously, prior to or after modification to express ER-Hoxb8 fusion protein and/or inhibit the expression of SIRPα. In another embodiment, a bone marrow cell is exposed to the targeting antibody simultaneously, prior to or after modification to express ER-Hoxb8 and/or inhibit the expression of SIRPα.

In some embodiments, the targeting antibody binds to an antigen on a target cell. Examples of antigens include cell surface markers that are associated with viral, bacterial and parasitic infections, autoimmune disease, and cancer cells.

The choice of targeting antibody depends upon the type and number of antigens that are present on the surface of a target cell. For example, the targeting antibody may be chosen to recognize an antigen that acts as a cell surface marker on a target cell associated with a particular disease state.

In one embodiment, the targeting antibody binds to a tumor antigen, such as an antigen that is specific for a tumor or cancer of interest. In one embodiment, the tumor antigen of the present invention comprises one or more antigenic cancer epitopes. Nonlimiting examples of tumor associated antigens include CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tyrosinase-related protein 1 (Tyrp1); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlep(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In one embodiment, the targeting antibody is a tumor specific antibody.

In certain embodiments, the targeting antibody is a fragment of an antibody, such as the antigen binding fragment. The targeting antibody can include any domain that binds to the antigen and may include, but is not limited to, a monoclonal antibody, a polyclonal antibody, a synthetic antibody, a human antibody, a humanized antibody, a non-human antibody, a single chain antibody, a single chain variable fragment, a chimeric antibody, and any fragment thereof. Thus, in one embodiment, the targeting antibody comprises at least a portion of a mammalian antibody.

In some instances, the targeting antibody is derived from the same species in which it will ultimately be used in. For example, for use in humans, the targeting antibody comprises a human antibody, a humanized antibody, or a fragment thereof.

### Human Antibodies

It may be preferable to use human antibodies or fragments thereof when using the targeting antibody. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Humanized Antibodies

Alternatively, in some embodiments, a non-human antibody can be humanized, where specific sequences or regions of the antibody are modified to increase similarity to an antibody naturally produced in a human. For instance, in the present invention, the antibody or fragment thereof may comprise a non-human mammalian scFv. In one embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp): 5973s-5977s (1995), Couto et al., Cancer Res., 55(8): 1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies. Humanization of antibodies can also be achieved by veneering or resurfacing (EP 592, 106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized that retain high affinity for the target antigen and that possess other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody. However, using certain methods of humanization, the affinity and/or specificity of binding of the antibody to the target antigen may be increased using methods of “directed evolution,” as described by Wu et al., J. Mol. Biol., 294:151 (1999), the contents of which are incorporated herein by reference herein in their entirety.

### Compositions

The present invention includes a modified phagocytic cell or progenitor cell thereof that possesses phagocytic activity against tumor tissue. In one embodiment, the phagocytic cell or precursor cell thereof is modified by expressing an ER-Hoxb8 fusion protein, reduced expression of signal regulatory protein-alpha (SIRPα) protein and bound to a targeting antibody.

In some embodiment, the phagocytic cell is a macrophage modified by expressing an ER-Hoxb8 fusion protein, reduced expression of signal regulatory protein-alpha (SIRPα) protein and bound to a targeting antibody.

In some embodiments, a bone marrow cell is modified by expressing an ER-Hoxb8 fusion protein, reduced expression of signal regulatory protein-alpha (SIRPα) protein and bound to a targeting antibody. In one embodiment, the modified bone marrow cell differentiates into a mature modified macrophage. In another embodiment, the differentiated modified macrophage has a stronger phagocytic activity than a native macrophage of the mammal.

In one aspect, the invention includes a composition comprising the modified macrophage or precursor cell thereof as described herein. In another aspect, the invention includes a composition comprising a modified bone marrow cell bound to a targeting antibody.

The composition also has a therapeutic effect on a tumor tissue. In one embodiment, the therapeutic effect comprises tumor shrinkage of at least about 60% of the tumor. In some embodiments, the tumor tissue comprises a brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, uterine cancer, and the like.

### Sources of Cells

In one embodiment, a source of phagocytic cells or precursor cells thereof used in the compositions and methods described herein is obtained from a subject. Non-limiting examples of subjects include humans, dogs, cats, mice, rats, and transgenic species thereof. Preferably, the subject is a human. The cells can be obtained from a number of sources, including peripheral blood mononuclear cells, bone marrow, lymph node tissue, spleen tissue, umbilical cord, and tumors. In certain embodiments, any number of cell lines available in the art may be used. In certain embodiments, the cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, cells are isolated from peripheral blood by lysing the red blood cells and depleting the lymphocytes and red blood cells, for example, by centrifugation through a PERCOLL™ gradient. Alternatively, cells can be isolated from umbilical cord, bone marrow, spleen, lymph nodes, thymus, ascites fluid, tumor, or other source of phagocytic cells. In any event, a specific subpopulation of cells can be further isolated by positive or negative selection techniques.

The mononuclear cells so isolated can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of specific cell populations by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, enrich of a cell population for monocytes, macrophages and/or dendritic cells by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In one embodiment, a population of cells include, but are not limited to, peripheral blood mononuclear cells, cord blood cells, a purified population of monocyte progenitor cells, a purified population of phagocytic cells, and a cell line. In another embodiment, peripheral blood mononuclear cells comprise the population of phagocytic cells. In another embodiment, monocyte progenitor cells comprise the population of phagocytic cells. In yet another embodiment, purified cells comprise the population of phagocytic cells.

The present invention includes the composition further comprising opsonins. Opsonins include any molecule or compound that enhance phagocytosis of a target. Coating a target cell, such as a cancerous, necrotic or other target cell, enhances selective phagocytosis by phagocytic cells, such as macrophages, monocytes, neutrophils, mast cells, and dendritic cells. The phagocytic cell expresses opsonin receptors, such as Fc and complement receptors, which bind the opsonin to activate the phagocytic process. Opsonins include, but are not limited to, IgG antibody, red blood cell (anti-RBC) antibody, IgM, C3b, C4b, iC3b, mannose-binding lectin, C-reactive protein, and any combination thereof.

In another aspect, the invention includes a composition comprising a modified bone marrow cell and an opsonin, wherein the composition has a therapeutic effect on a tumor tissue.

In one embodiment, the modified bone marrow cell differentiates into a mature modified macrophage. In another embodiment, the differentiated mature modified macrophage has a stronger phagocytic activity than the native macrophage of the mammal.

In embodiments where the tissue is a tumor tissue, the composition has a therapeutic effect on the tumor tissue. In one embodiment, the therapeutic effect comprises tumor shrinkage of at least about 60% of the tumor. In another embodiment, the tumor shrinkage is at least 20%, 25%, 30%, 35%, 40%, 45%, 50%, 55%, 60%, 65%, 70%, 75%, 80%, 85%, 90%, 95%, or greater or any percentage therebetween.

### Methods of Production

In one aspect, the invention includes a method of producing a modified phagocytic cell or precursor cell thereof, the method comprising obtaining a macrophage precursor cell from a subject, transducing the precursor cell with an expression vector encoding a fusion protein comprising an estrogen receptor ligand binding domain and a Hoxb8 protein, contacting the precursor cell with a estrogen receptor ligand to reversibly immortalize the precursor cell, modifying an endogenous genetic locus encoding a SIRPα protein, differentiating the precursor cell into a macrophage by withdrawal of the ER ligand and culture in the presence of an effective amount of stem cell factor (SCF) and granulocyte-macrophage colony-stimulating factor (GM-CSF), and coating the Fc receptors with at least one antibody specific for an antigen on a target cell.

In one embodiment, the modified phagocytic cell is a macrophage or a monocyte. In another embodiment, the phagocytic cell is a bone marrow cell, such as a bone marrow cell that differentiates into a mature macrophage.

In another embodiment, the modified phagocytic cell is contacted with targeting antibody simultaneously, prior to or after modification of SIRPα expression.

In yet another embodiment, the modified phagocytic cell is modified to have a stronger phagocytic activity than the native phagocytic cell of the mammal.

In one example of the invention, the method described herein comprises altering, interfering, repressing, or reducing SIRPα expression. Repressors of SIRPα expression include, but are not limited to, SIRPα-shRNA, SIRPα-siRNA, a CRISPR system targeted to SIRPα, and a combination thereof.

The method further comprises administering an opsonin or targeting antibody in the composition. Examples of opsonins include, but are not limited to, IgG antibody, red blood cell (anti-RBC) antibody, IgM, and any combination thereof. In one embodiment of the invention, the modification of SIRPα expression occurs prior to administration of the opsonin. In another embodiment, the modification of SIRPα expression occurs with the opsonin. In yet another embodiment, the modification of SIRPα expression occurs after the administration of the opsonin.

In another embodiment, the tissue is a tumor tissue. In embodiments where the tissue is a tumor tissue, the therapeutic effect includes, but is not limited to, the removal or elimination of tumor tissue or a decrease in tumor size or tumor tissue. The effective amount may cause the tumor tissue to be decreased when compared to the original tissue size, the tumor tissue size prior to therapeutic treatment, a reference tissue or any other standard or condition. In one embodiment, the method comprises tumor shrinkage of at least about 60% of the tumor. In another embodiment, the tumor shrinkage is at least 20%, 25%, 30%, 35%, 40%, 45%, 50%, 55%, 60%, 65%, 70%, 75%, 80%, 85%, 90%, 95%, or greater or any percentage therebetween.

In another aspect, the invention includes a method of modulating phagocytic activity to target a specific tissue in a mammal. The method comprises administering to the mammal an effective amount of a composition comprising a modified bone marrow cell, wherein the effective amount of the composition modulates phagocytic activity and has a therapeutic effect on the tissue in the mammal.

In one embodiment, the modified bone marrow cell differentiates into a mature modified macrophage. In another embodiment, the differentiated mature modified macrophage has a stronger phagocytic activity than the native macrophage of the mammal. In yet another embodiment, the modified bone marrow cell may be an autologous or allogenic bone marrow cell.

In certain embodiments, the nucleic acid encoding the ER-Hoxb8 fusion protein and/or SIRPα targeting CRISPR system can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host can be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) can be used to introduce the nucleic acid into a host cell. Accordingly, an expression vector (e.g., a lentiviral vector) of the present disclosure can comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter can increase the efficiency in expression of downstream transgenes. Physiologic promoters (e.g., an EF-1α promoter) can be less likely to induce integration mediated genotoxicity and can abrogate the ability of the retroviral vector to transform stem cells. Other physiological promoters suitable for use in a vector (e.g., lentiviral vector) are known to those of skill in the art and can be incorporated into a vector of the present disclosure. In some embodiments, the vector (e.g., lentiviral vector) further comprises a non-requisite cis acting sequence that can improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and can be incorporated into a vector (e.g., lentiviral vector) of the present disclosure. In some embodiments, the vector further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present disclosure further comprises a WPRE sequence. Various posttranscriptional regulator elements are known to those of skill in the art and can be incorporated into a vector (e.g., lentiviral vector) of the present disclosure. A vector of the present disclosure can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present disclosure includes a 3′ U3 deleted LTR. Accordingly, a vector (e.g., lentiviral vector) of the present disclosure can comprise any combination of the elements described herein to enhance the efficiency of functional expression of transgenes. For example, a vector (e.g., lentiviral vector) of the present disclosure can comprise a WPRE sequence, cPPT sequence, RRE sequence, 5′LTR, 3′ U3 deleted LTR′ in addition to a nucleic acid encoding for a fusion protein.

Vectors of the present disclosure can be self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

In some embodiments, a nucleic acid of the present disclosure can be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15: 9053. Introducing RNA comprising a nucleotide sequence encoding a polypeptide of the present disclosure into a host cell can be carried out in vitro, ex vivo or in vivo. For example, a host cell (e.g., a phagocytic cell, a macrophage, a monocyte progenitor cell, etc.) can be electroporated in vitro or ex vivo with RNA comprising a nucleotide sequence encoding a fusion protein and/or SIRPα targeting CRISPR system of the present disclosure.

In order to assess the expression of a fusion protein or SIRPα targeting CRISPR system or portions thereof, the expression vector to be introduced into a cell can also contain either a selectable marker gene or a reporter gene, or both, to facilitate identification and selection of expressing cells from the population of cells sought to be transfected or infected through viral vectors. In some embodiments, the selectable marker can be carried on a separate piece of DNA and used in a co-transfection procedure. Both selectable markers and reporter genes can be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes can include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### Methods of Treatment

The compositions described herein may be formulated as a therapy. The pharmaceutical composition may also include a pharmaceutically acceptable carrier. A therapeutically effective amount of the pharmaceutical composition may be administered.

In one aspect, the invention includes a method of treating cancer in a subject in need thereof, comprising obtaining a monocyte precursor cell from a subject, transducing the precursor cell with an expression vector encoding a fusion protein comprising an estrogen receptor ligand binding domain and a Hoxb8 protein, contacting the precursor cell with a estrogen receptor ligand to reversibly immortalize the precursor cell, modifying an endogenous genetic locus encoding a SIRPα protein, terminally differentiating the precursor cell into a modified phagocytic cell by withdrawal of the ER ligand and culture in the presence of an effective amount of stem cell factor (SCF) and granulocyte-macrophage colony-stimulating factor (GM-CSF), coating the Fc receptors of the modified macrophage with at least one targeting antibody specific for an antigen on a target cell, and administering an effective amount of the modified phagocytic cell to the subject, thereby treating the cancer.

In one embodiment, the modified phagocytic cell is a macrophage or a monocyte. In another embodiment, the modified phagocytic cell is a bone marrow cell that differentiates into a mature modified macrophage. In another embodiment, the differentiated mature modified macrophage has a stronger phagocytic activity than the native macrophage of the mammal. In yet another embodiment, the modified phagocytic cell may be an autologous or allogenic to the mammal.

In certain embodiments, the method further comprises administering to the subject an effective amount of an opsonin specific for the cancer cells. In certain embodiments, the opsonin is the targeting antibody used to produce the modified phagocytic cell or precursor cell thereof.

In other embodiments, the opsonin is different from the targeting antibody. In still other embodiments, the opsonin includes the targeting antibody used to produce the modified phagocytic cell or precursor cell thereof and other opsonins specific for the cancer cells or antigens expressed by the cancer cells.

In certain embodiments, the composition of the invention provides a therapeutic effect. Therapeutic effects include, but are not limited to, removal or elimination of target cells, decrease in tumor cells or tumor size or tumor tissue, removal or elimination of necrotic or apoptotic cells, or decrease in necrotic or apoptotic cells by phagocytosis. The effective amount may cause the target cells to be decreased in number when compared to the original tissue size, such as a tumor tissue, the tissue size prior to therapeutic treatment, a reference tissue or any other standard or condition.

In one embodiment, the treatment of the tumor comprises at least one selected from the group consisting of suppression of tumor tissue growth and decrease of the tumor tissue by at least 60%. In another embodiment, the tumor tissue is decreased by at least 20%, 25%, 30%, 35%, 40%, 45%, 50%, 55%, 60%, 65%, 70%, 75%, 80%, 85%, 90%, 95%, or greater. In still another embodiment, the tumor tissue is decreased by at least 60% as compared to an original size of the tumor tissue, the tumor tissue size prior to therapeutic treatment, a reference tissue or any other standard or condition. In another embodiment, the tumor tissue is decreased by at least 20%, 25%, 30%, 35%, 40%, 45%, 50%, 55%, 60%, 65%, 70%, 75%, 80%, 85%, 90%, 95%, or greater as compared to an original size of the tumor tissue.

In one embodiment, the target cells include, but are not limited to, tumor cells, such as malignant and metastatic cells, necrotic cells, apoptotic cells, infected cells, such as bacterial or viral infected cells, and other target cells. In one embodiment, the target cells are in a tumor tissue, such as a brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, uterine cancer, and the like.

The composition of the invention can be administered in dosages and routes and at times to be determined in appropriate pre-clinical and clinical experimentation and trials. The compositions may be administered multiple times at dosages within these ranges.

In certain embodiments, the composition of the invention provides a therapeutic effect. Therapeutic effects include, but are not limited to, removal or elimination of target cells, decrease in tumor cells or tumor size or tumor tissue, removal or elimination of necrotic or apoptotic cells, or decrease in necrotic or apoptotic cells by phagocytosis. The effective amount may cause the target cells to be decreased in number when compared to the original tissue size, such as a tumor tissue, the tissue size prior to therapeutic treatment, a reference tissue or any other standard or condition.

In one embodiment, the treatment of the tumor comprises at least one selected from the group consisting of suppression of tumor tissue growth and decrease of the tumor tissue by at least 60%. In another embodiment, the tumor tissue is decreased by at least 20%, 25%, 30%, 35%, 40%, 45%, 50%, 55%, 60%, 65%, 70%, 75%, 80%, 85%, 90%, 95%, or greater. In still another embodiment, the tumor tissue is decreased by at least 60% as compared to an original size of the tumor tissue, the tumor tissue size prior to therapeutic treatment, a reference tissue or any other standard or condition. In another embodiment, the tumor tissue is decreased by at least 20%, 25%, 30%, 35%, 40%, 45%, 50%, 55%, 60%, 65%, 70%, 75%, 80%, 85%, 90%, 95%, or greater as compared to an original size of the tumor tissue.

In one embodiment, the target cells include, but are not limited to, tumor cells, such as malignant and metastatic cells, necrotic cells, apoptotic cells, infected cells, such as bacterial or viral infected cells, and other target cells. In one embodiment, the target cells are in a tumor tissue, such as a brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, uterine cancer, and the like.

The composition of the invention can be administered in dosages and routes and at times to be determined in appropriate pre-clinical and clinical experimentation and trials. The compositions may be administered multiple times at dosages within these ranges.

Administration of the composition of the invention may be combined with other methods useful to treat the desired disease or condition as determined by those of skill in the art.

The administration of the composition of the invention may be carried out in any convenient manner known to those of skill in the art. The composition of the present invention may be administered to a mammal by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the composition of the invention is injected directly into a target site in the mammal, a local disease site in the mammal, a lymph node, an organ, a tumor, and the like. In one embodiment, the composition described herein is administered intravenously to the mammal.

In one embodiment, the modified phagocytic cell or precursor cell thereof is administered intravenously to the mammal. The modified phagocytic cell or precursor cell thereof may be administered prior to administration of the opsonin, with the opsonin or after the administration of the opsonin.

### Pharmaceutical Compositions

Pharmaceutical compositions of the invention of the present disclosure may comprise the composition as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

It can generally be stated that a pharmaceutical composition comprising the modified macrophages or precursor cells thereof described herein may be administered at a dosage of 104 to 10⁹ cells/kg body weight, preferably 10⁵ to 10⁶ cells/kg body weight, including all integer values within those ranges. The compositions may also be administered multiple times at these dosages. The composition can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer the modified phagocytic cells or precursor cells thereof to a subject and then subsequently reinfuse the patient with additional modified phagocytic cells or precursor cells thereof. This process can be carried out multiple times every few weeks. In certain embodiments, repressed phagocytic cells can be obtained from blood draws of from 10 ml to 400 ml. In certain embodiments, repressed phagocytic cells are obtained from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of cells.

In certain embodiments of the present invention, methods of treatment using the compositions described herein, or other methods known in the art where composition are administered to a patient in conjunction with (e.g., before, simultaneously or following) any number of relevant treatment modalities. In further embodiments, the compositions of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In a further embodiment, the compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In certain embodiments, the compositions described herein may be used for the manufacture of a medicament for the treatment of response disease or condition in a subject in need thereof. In yet other embodiments, the compositions described herein may be used for the manufacture of a medicament for the treatment of a cancer in a subject in need thereof.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002).

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in the experiments disclosed herein are now described.

Cell culture. All cell cultures were maintained in a humidified incubator at 37° ° C., 5% CO2. Basal media were supplemented with 10% (v/v) fetal bovine serum (FBS, Sigma F2442), 100 U/mL penicillin and 100 μg/mL streptomycin (1% P/S, Gibco 15140122). B16-F10 (CRL-6475) cells were obtained from American Type Culture Collection (ATCC) and cultured at in either RPMI-1640 (Gibco 11835-030) or DMEM (Corning 10-013-CV or Gibco 11995-065). B16 KO cell lines were generated as described previously using single guide RNA (sgRNA) constructs targeting CD47 (5′-TCCCCGTAGAGATTACAATG (SEQ ID NO: 1)) and Tyrp1 (5′-CTTGTGGCAATGACAAATTG (SEQ ID NO: 2)) and cultured under the same conditions as the parental wild-type B16-F10 cell line. J774A. 1 mouse macrophages (ATCC TIB-67) and TC-1 cells (generously provided by Dr. Sunil Singhal from the University of Pennsylvania) were both cultured in RPMI-1640. YUMM2.1 cells (a gift from Dr. Chi Van Dang at the Wistar Institute) and CT-2A EGFRvIII cells (described previously 2) were cultured in DMEM. RM-9 (ATCC CRL-3312) were cultured in DMEM:F12 (Gibco 11320-033).

Antibodies. Antibodies used for in vivo treatment and blocking, in vitro phagocytosis, and flow cytometry are as follows: anti-mouse/human Tyrp1 clone TA99 (BioXCell BE0151), isotype IgG2a control clone C1.18.4 (BioXCell, BE0085), anti-mouse CD47 clone MIAP301 (BioXCell BE0270), isotype control rat IgG2a (BioXCell, BE0089), anti-mouse SIRPα clone P84 (BioLegend 144004), isotype control rat IgG1 (BioLegend, 400414), anti-GD2 clone 14G2a (BioXCell BE0318). Low-endotoxin and preservative-free antibody preparations were used for in vivo opsonization and blocking and in vitro phagocytosis experiments. In some experiments, cells were opsonized with anti-mouse RBC IgG or antiserum formats (Rockland 210-4139 or 110-4139). The following BioLegend antibodies were used for flow cytometry staining: APC anti-mouse I/A-I-E clone M5/114.15.2 (107614), PerCP/Cy5.5 anti-mouse CD206 clone C068C2 (141715), FITC anti-mouse CD64 clone X54-5/7.1 (139315), PE-Dazzle594 anti-mouse CD32 clone S17012B (156411), PE/Cy7 anti-mouse CD16 clone S17014E (158015), AF647 anti-mouse CD16.2 clone 9E9 (149525), PE anti-Ly6C clone HK1.4 (128007), PE anti-F4/80 clone BM8 (123109), APC/Cy7 anti-F4/80 clone BM8 (123118), APC anti-mouse CD45.2 clone 104 (109814), PE/Cy7 anti-mouse/human CD11b clone M1/70 (101216), FITC anti-mouse SIRPα clone P84 (144006), APC/Cy7 anti-mouse IgG1 clone RMG1-1 (406619), APC anti-mouse IgG2a clone RBG2a-62 (407110), PE/Cy7 anti-mouse IgG2b clone RMG2b-1 (406714), biotin anti-mouse IgG3 clone RMG3-1 (406803). Other flow cytometry detection was performed with PE streptavidin (BioLegend 405203), Alexa Fluor (AF) 647 donkey anti-mouse IgG (Invitrogen A31571), and AF647 goat anti-rat IgG (Invitrogen A21247). For cultured myeloid cells or disaggregated tumor cell suspensions, Fc-receptors were blocked with rat anti-mouse CD16/32 (clone 2.4G2, BD 553149) and rat anti-mouse CD16.2 (clone 9E9, BioLegend 149502) prior to staining, except in the case of panels for Fc-receptors. Primary antibodies used in Western blotting or immunofluorescence (IF) microscopy were anti-β-actin clone C4 (Santa Cruz sc 47778), mouse anti-lamin-A/C clone 4C11 (Cell Signaling Technology 4777), rabbit anti-lamin-B1 (Abcam ab16048), mouse anti-N-cadherin clone 13A9 (Biolegend 844701), and mouse anti-α-tubulin (Sigma T9026). Secondary antibodies used in Western blotting and IF were sheep anti-mouse IgG HRP (GE Life Sciences NA931V), goat anti-mouse IgG IRDye800CW (LiCOR 926-32210), AF488 donkey anti-mouse IgG (Invitrogen A21202), and AF647 donkey anti-rabbit IgG (Invitrogen A31573).

Mice. C57BL/6J mice (Jackson Laboratory 000664) were 6-12 weeks old at the time of first challenge unless otherwise specified. Age-matched mice were used as controls in rechallenge experiments. NOD-scid IL2Rγ^{nul} (NSG) mice aged 6-12 weeks old were obtained from the Stem Cell & Xenograft Core at the University of Pennsylvania. All experiments were performed in accordance with protocols approved by the Institutional Animal Care and Use Committee of the University of Pennsylvania.

Bone marrow-derived macrophages (BMDMs). Bone marrow was harvested from donor mice, lysed with ACK buffer (Gibco A1049201) to deplete red blood cells, and cultured on Petri dishes for 7 days in IMDM (Gibco 12440053) supplemented with 10% FBS, 1% P/S, and 20 ng/ml recombinant mouse macrophage colony-stimulating factor (M-CSF, BioLegend 576406). Successful differentiation was confirmed by flow cytometry staining with antibodies against macrophage markers. Cytokine-primed BMDMs were cultured in RPMI growth media+20 ng/ml M-CSF+20 ng/mL IFNγ (BioLegend 575302) or 20 ng/ml IL-4 (BioLegend 574302) for 48 h prior to use in tumoroid assays or prior to analysis of protein expression by flow cytometry. For immunofluorescence microscopy, differentiated BMDMs were detached and re-plated on bare glass coverslips at a density of ˜5.5×10³ per cm2 in RPMI growth media+20 ng/ml M-CSF. Cytokines were added 3 h later when cells were mostly attached to the glass and fixed and stained 48 h later.

Conditionally immortalized macrophage (CIM) progenitors. Macrophage progenitors were generated as previously described. CIMs were cultured in suspension in 10 cm Petri dishes in RPMI-1640 (Gibco A1049101) supplemented with 10% FBS and 1% P/S as above. Media was also supplemented with 2 μM β-estrogen (Sigma Aldrich E2758-250MG) and 10 ng/ml recombinant mouse GM-CSF (BioLegend 576302). Cells were passaged every 2 days at sub-confluent concentration of 1×105 cells/mL. To differentiate for phagocytosis assays, cells were washed twice with 5% FBS/PBS to remove excess β-estrogen and plated in supplemented DMEM (10% FBS/1% PS) with 20 ng/mL recombinant mouse M-CSF for 7 days.

In vitro phagocytosis. For 2D phagocytosis assays, BMDMs were detached and re-plated in 24 well plates at a density of 1.8×104 per cm2 in IMDM supplemented with 10% FBS, 1% P/S, and 20 ng/mL M-CSF. The next day, BMDMs were labeled with 0.5 μM CellTracker Deep Red dye (Invitrogen C34565) according to the manufacturer's protocol. Following staining, macrophages were washed and incubated in serum-free media supplemented with 0.1% (w/v) bovine serum albumin. In some experiments, target cells were labeled with carboxyfluorescein diacetate succinimidyl ester (Invitrogen V12883) also according to the manufacturer's protocol. B16 were detached and opsonized with 10-20 μg/mL anti-Tyrp1, with mouse IgG2a isotype control antibody, or with 5% (v/v) mouse serum collected during the course of tumor challenge as described below in serum-free media for 30 min on ice. For CD47 blockade experiments, 20 μg/mL MIAP301 or rat IgG2a isotype control antibody was added during opsonization. Opsonized B16 suspensions were added to BMDMs at a ˜2:1 ratio and incubated at 37° C., 5% CO2 for 2 h. Non-adherent cells were removed by washing with PBS and remaining cells were fixed with 4% formaldehyde, counterstained with Hoechst 33342 (Invitrogen H3570), and imaged on an Olympus IX inverted microscope with a 40×/0.6 NA or 20×/0.4 NA objective. The Olympus IX microscope was equipped with a Prime sCMOS camera (Photometrics), and a pE-300 LED illuminator (CoolLED), which were controlled with MicroManager software v1.4 or v2.5.

To visualize SIRPα on phagocytic macrophages, assays were performed similarly using J774A.1 mouse macrophages to which mouse red blood cells opsonized with rabbit anti-mouse RBC were added. Staining for SIRPα and phospho-tyrosine was performed as previously described.

Tumoroids. To generate surfaces conducive to B16 tumoroid formation, 96-well plates (Greiner Bio-One 650161) were either coated with 70 μL of 2% agarose in water or PBS or, more commonly, treated for 20 min with 100 μL of anti-adherence rinsing solution (StemCell Technologies 07010). The wells were washed with PBS, and then blocked with 0.5-1% (w/v) bovine serum albumin (BSA, Sigma) 37° ° C. for 1 h. B16 were detached by brief trypsinization, resuspended in RPMI supplemented with 10% FBS, 1× non-essential amino acid solution (Gibco 11140050) and 1 mM sodium pyruvate (Gibco 11360070) at a concentration of 1×103 or 1×104 per mL, and added to passivated wells in a volume of 100 μL. A single aggregate of B16 cells formed in each well, typically within 12 h. For tumoroid disaggregation studies, EDTA and EGTA were added in PBS to obtain a final concentration of 2 mM, and in some cases magnesium sulphate was also added for a final concentration of 5 mM Mg2+. Latrunculin A (Invitrogen L12370) dissolved at 1 μM in DMSO was further diluted in PBS and added to obtain a final concentration of 1 μM. For phagocytosis versus growth studies, CellTracker Deep Red-labeled BMDMs and antibodies (final concentration 20 μg/mL anti-Tyrp1 and/or 20 μg/mL anti-CD47 or 10 μg/mL anti-SIRPα) or mouse serum (final concentration 1:200) were added 24 h later in a volume of 20 μL RPMI growth media with 120 ng/mL M-CSF. For myosin-II inhibition studies, 20 μM blebbistatin (MilliporeSigma 203389) or an equal volume of DMSO vehicle was added to B16 tumoroids for 1 h prior to addition of BMDMs. Tumoroids were imaged on the Olympus IX microscope with 20×/0.4 NA, 10×/0.3 NA, or 4×/0.13 NA objectives. Tumoroid areas were measured in Fiji/ImageJ after thresholding GFP fluorescence using built-in thresholding algorithms and manually adjusting the threshold as needed.

For confocal imaging, clusters were fixed ˜20 h after addition of BMDMs and antibodies by adding an equal volume of 4% formaldehyde directly to the media in each well for 30 min at room temperature. Half of this solution was removed and replaced with an equal volume of 2% formaldehyde for 30 min. The clusters were washed with PBS to remove fixative, repeating the procedure of removing and replacing half the volume at each step. The clusters were transferred via a wide-bore pipette tip to an 8-well chambered cover glass (Lab-Tek) and counterstained with 5 μg/mL Hoechst 33342. Confocal images were acquired on a Leica TCS SP8 with a 20×/0.75 NA objective. Radial fluorescence profiles were calculated in ImageJ by thresholding max intensity projections of GFP and Deep Red fluorescence and calculating the tumoroid centroid, which was used as the input for r=0 in the Radial Profile ImageJ plugin applied individually to the GFP and Deep Red channels. This plugin calculates the average fluorescence intensity I(r) for a circle of radius r centered at the inputted position of the tumoroid centroid. I(r) values were normalized by dividing by the total average fluorescence intensity ΣI/(r) for all r.

Entropy image analysis. Fluorescence images of CellTracker Deep Red-labeled macrophages were converted to binary images of black cells on a white background using the Otsu threshold algorithm in ImageJ. The number of dark pixels was determined from the image histogram, and the image was saved as a PNG file, which is a form of lossless file compression to determine the compressed file size as a measure of entropy. Random and structured images were generated and analyzed using the Python NumPy and PIL/Pillow packages, respectively.

Transcriptomic analysis. Public microarray data for BMDMs treated with IFNγ for 18 h versus control BMDMs (GSE60290) and for BMDMs treated with IL-4 for 24 h versus control BMDMs (GSE69607) were accessed through the NCBI GEO repository and analyzed using the GEO2R interactive web tool. Differential expression was considered significant for p<0.05, where the adjusted p-values were computed by the default method in GEO2R that uses the false discovery rate method of Benjamini and Hochberg. The B16-F10 RNAseq dataset (GSE162105) analyzed for cell adhesion genes was also accessed through the GEO repository.

Flow cytometry. Phagocytosis of B16 by macrophages in tumoroids was assessed by pooling identically treated tumoroids from a single 96-well plate, disaggregating them to single cell suspensions, and analyzing cell suspensions by flow cytometry. Tumoroids were collected into FACS buffer (PBS plus 1% BSA and 0.1% sodium azide) ˜18 h after addition of BMDMs±anti-Tyrp1, pelleted by centrifugation and resuspended in FACS buffer+0.5 mM EDTA for 15-30 min at room temperature. The suspension was pipetted up and down until the cells were deemed to be disaggregated by inspection on a hematocytometer. Viable cells were distinguished by staining with Zombie aqua fixable viability dye (BioLegend 423101) in PBS for 15 min at room temperature. The cells were washed with FACS buffer, fixed with 4% formaldehyde for 15 min at room temperature, and stored at 4° C. until analysis. Flow cytometry was performed on a BD LSRII (Benton Dickinson) and data were analyzed with FCS Express 7 software (De Novo Software). Doublets, cell debris, and nonviable cells were excluded and GFP+ DeepRed+ events were considered to be phagocytic macrophages.

For mAb and serum binding analyses, B16, YUMM2.1, or CT-2A cells were detached, washed, and resuspended in FACS buffer containing primary antibody or 5% (v/v) mouse serum collected as described below. Cell suspensions were incubated at 4° C. for 30 min and agitated to prevent cell settling. Cells were washed three times with FACS buffer and incubated with fluorophore-conjugated secondary antibodies in FACS buffer for 30 min at 4° C. and later with PE-streptavidin if required to label biotinylated anti-mouse IgG3. Finally, cells were washed three times and resuspended in FACS buffer containing 0.2 μg/mL DAPI (Cell Signaling Technology). For samples that could not be analyzed on the day of staining and required fixation, cells were stained with Zombie aqua fixable viability dye in PBS for 15 min at room temperature prior to staining with antibodies or serum, and DAPI was omitted from the final fixed cell suspension. Those samples were fixed with FluoroFix Buffer (BioLegend 422101) or 2-4% paraformaldehyde. For experiments analyzed in the same plot, the same lots of primary and secondary antibodies and identical staining conditions were used. When these analyses were performed on different days, UltraRainbow calibration beads (Spherotech URCP-38-2K) were used to adjust the photomultiplier tube voltage in each channel to maintain the median fluorescence intensity of the brightest peak within a tolerance of ±5%.

Tumor models. B16, RM-9, and TC-1 cell lines cultured in supplemented growth media were detached by brief trypsinization, washed with PBS, and resuspended at 2×106 per mL in PBS. Cell suspensions remained on ice until injection. Fur on the injection site (usually the right flank) was wet slightly with a drop of 70% ethanol and brushed aside to visualize the skin. A 100 μL bolus (containing 2×105 tumor cells) was injected beneath the skin. Treated mice with B16 tumors received i.v. injections of anti-Tyrp1 clone TA99 (250 μg antibody in 100 μl PBS) via the lateral tail vein on days 4, 5, 7, 11, 13, and 15 post tumor cell inoculation. Where indicated, mice also received i.v. injections of anti-CD47 clone MIAP301 (83 μg antibody dosed in the same in 100 μl PBS volume as anti-Tyrp1). Mice with RM-9 tumors received 250 μg (in 100 μl PBS) anti-GD2 clone 14G2a i.v. on the same schedule instead of anti-Tyrp1. Tumors were monitored by palpation and measured with digital calipers. The projected area was roughly elliptical and calculated as A=π/4×L×W where L is the length along the longest axis and W is the width measured along the perpendicular axis. A projected area of 125 mm² was considered to be the terminal tumor burden for survival analyses.

Serum collection. Blood was drawn retro-orbitally and allowed to clot for 30-60 min at room temperature in a microcentrifuge tube. The serum was separated from the clot by centrifugation at 1500×g and stored at −20° C. for use in flow cytometry, phagocytosis assays, and Western blotting.

Immune infiltrate analysis of tumors. For flow cytometry of immune cell markers in tumors, mice were treated with a single dose of i.v. anti-Tyrp1 at day 4 (96 h) post inoculation and sacrificed 24 h later. Tumors were photographed, then excised and placed into 5% FBS/PBS. Tumors were then disaggregated with Dispase (Corning 354235) supplemented with 3 mg/mL Collagenase Type IV (Gibco 17104019) and DNAsel (Sigma-Aldrich 10104159001) for 30 min at 37° C., centrifuged for 5 min at 300×g, and resuspended in 1 mL of ACK lysing buffer for 12 min at RT. Samples were centrifuged for 5 min at 300×g, washed with FACS buffer, and incubated in FACS buffer containing fluorophore-conjugated antibodies to immune markers on ice for 30 min. Samples were then washed with FACS buffer and fixed with FluoroFix Buffer for 30 min at RT prior to analysis on a flow cytometer.

For IF staining of tumor sections, mice were treated in the same manner. Whole tumors were then excised, fixed in 4% paraformaldehyde overnight at 4° C., and stored in 70% ethanol. The Comparative Pathology Core (University of Pennsylvania) embedded the tissues in paraffin, sectioned, and stained the tumor sections with anti-F4/80 according to their standardized protocols. Sections were imaged on an Olympus microscope as described above. Radial profile analysis was conducted with the Radial Profile Plot plugin for ImageJ. Sectioning and trichrome staining of B16 tumors were performed by the Molecular Pathology and Imaging Core (University of Pennsylvania). Tile scan images of the trichrome-stained tumor section were acquired on the EVOS FL Auto Imager with a 10×/0.25 NA objective (Thermo Fisher).

Adoptive cell transfers. Fresh bone marrow was harvested as above through the RBC lysis step. Marrow cells were then counted on a hemocytometer and resuspended at 8×107 cells/mL in 5% FBS/PBS. To block SIRPα, cells were then incubated with anti-SIRPα clone P84 (18 μg/mL) for 45 mins at room temperature on a rotator, centrifuged to remove unbound P84, and re-suspended again at 8×107 cells/mL in 2% FBS/PBS with or without 1 mg/mL anti-Tyrp1 (TA99). Marrow cells (2×107 cells in 250 μl 2% FBS/PBS) were injected i.v. into tumor-bearing mice 4 days after tumor engraftment. SIRPα KO CIM progenitors (4×106 cells in 250 μl 2% FBS/PBS with 1 mg/mL anti-Tyrp1 or anti-GD2) were injected i.v. in the same manner.

Western blotting. Lysate was prepared from B16 CD47 KO cells using RIPA buffer containing 1× protease inhibitor cocktail (Sigma P8340) and boiled in 1× NuPage LDS sample buffer (Invitrogen NP0007) with 2.5% (v/v) β-mercaptoethanol. For detection of N-cadherin, B16 proteins were fractionated by ultracentrifugation. Proteins were separated by electrophoresis in NuPAGE 4-12% Bis-Tris gels run with 1×MOPS buffer (Invitrogen NP0323) and transferred to an iBlot nitrocellulose membrane (Invitrogen IB301002). The membranes were blocked with 5% nonfat milk in Tris buffered saline (TBS) plus Tween-20 (TBST) for 1 h and stained with primary antibodies or with 5% (v/v) mouse serum overnight at 4° C. with agitation. The membranes were washed with TBST and incubated with 1:500 secondary antibody conjugated with horseradish peroxidase (HRP) or with 1:5000 secondary antibody conjugated with IRDye800CW in 5% milk in TBST for 1 h at room temperature with agitation. The membranes were washed again three times with TBST, then TBS. Membranes probed with HRP-conjugated secondary were developed with a 3,3′, 5,5′-teramethylbenzidine (TMB) substrate (Genscript L0022V or Sigma T0565). Developed membranes were scanned and analyzed with ImageJ. Membranes probed with IRDye800CW-conjugated secondary were imaged on an Odyssey near-infrared scanner (LiCor).

Pipette aspiration rheology. Tumoroids were formed in non-adhesive well-plates and transferred via wide-bore pipette to a glass-bottom dish (MatTek). Fresh tumors were harvested and stored in RPMI+10% FBS. The tumor was held in a chamber consisting of glass coverslips separated by silicone spacers on three sides and filled with RPMI+10% FBS. For aspiration of the tumor interior, the tumor was sliced with a scalpel or razor blade prior to placement in the chamber. A glass capillary (1 mm outer diameter/0.75 mm inner diameter, World Precision Instruments TW100-3) was pulled on a Browning/Flaming type pipette puller (Sutter Instruments P-97), scored with a ceramic tile, and broken to obtain micropipettes with diameters 40-100 μm. Only cleanly broken tips were used for aspiration. When required, micropipettes were bent with a De Fonbrune microforge. The micropipette was backfilled with 1% (w/v) BSA in PBS to prevent tissue adhesion and connected to a dual-stage water manometer. Aspiration was applied manually with a syringe (0.5-10 mL) and the pressure difference ΔP was measured with a calibrated pressure transducer (Validyne). Time-lapse brightfield microscopy of aspiration was performed on a TE300 microscope (Nikon) with a 20×/0.5 NA objective and images acquired on an Evolve Delta EMCCD camera (Photometrics) using MicroManager v1.4 or v2 software. Tissue elongation L(t) was determined by image analysis with ImageJ and fit to the standard linear solid model assuming a wall shape parameter Φ˜2. Tumor strain typically reached a plateau value after several seconds that was maintained for the short timescale of these experiments, allowing the elastic modulus to be approximated as E˜ΔP (L/RP)−1 as described previously. Tumoroid flow on longer timescales was modeled by including a viscosity term in the standard linear model.

Fluorescence recovery after photobleaching (FRAP). Confocal time-lapse imaging was performed at 37° ° C. with 5% CO2 in a humidified chamber with an SP8 inverted laser scanning confocal microscope (Leica). Time lapse images were acquired with a 63×/1.4NA oil immersion objective up to 10 min after photobleaching. Cells were imaged in phenol-red free low glucose DMEM (Gibco 11054-020) with 10% FBS and 1% P/S. Image sequences were analyzed using Fiji/ImageJ.

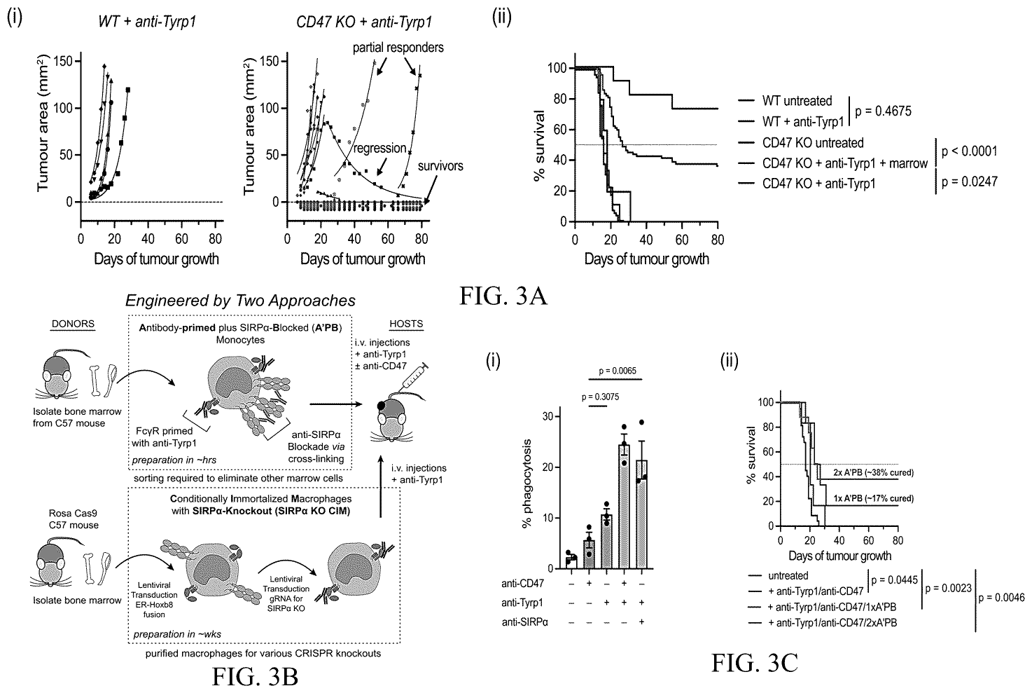

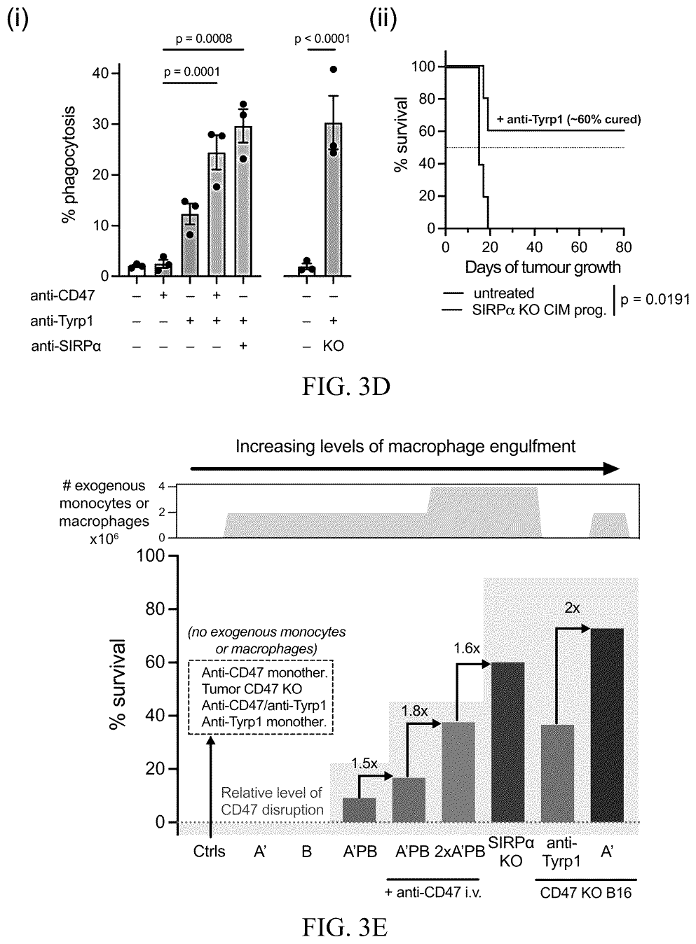

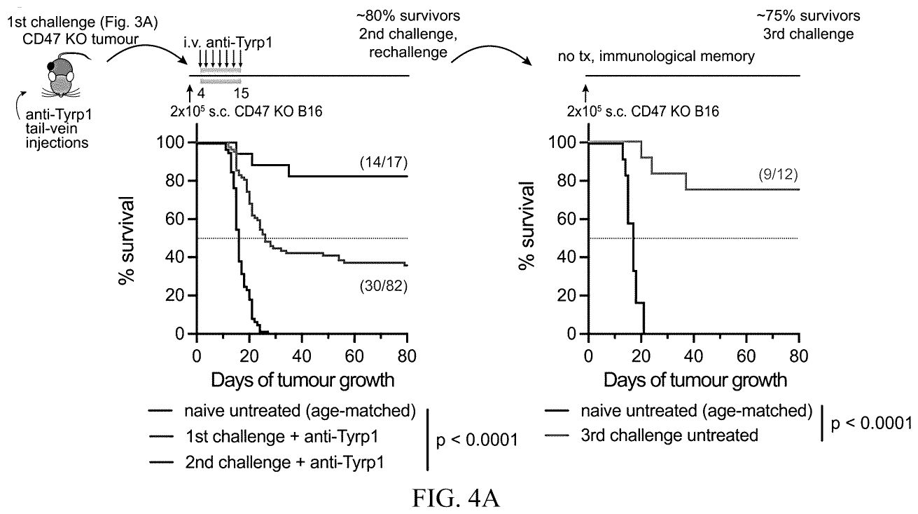

Statistical analysis and curve fitting. Statistical analyses and curve fitting were performed in Prism v8.4-v9.4 (GraphPad) and MATLAB R2020a (MathWorks). Details for each analysis are provided in figure legends, and exact p values are provided. Tumoroid and tumor growth data (projected area vs. time) were fit to the exponential growth model (A=A0 ekt for tumors and A=A1 ek(t−1) for tumoroids) using nonlinear least squares regression with prefactors A0 or A1, and k, the exponential growth rate. Outliers in samples of the fitted parameter k (≤1 outlier per condition, sample sizes ≥24) were identified by ROUT's method (maximum false discovery rate, Q=1%) in Prism. Cleaned data were fit to mathematical models described in Extended Data FIG. 3 for the dependence of k on macrophage number.

The results of the experiments are now described in the following examples.

### Example 1: Macrophages Cluster when Eradicating Tumoroids

To determine the requirements for macrophages to eliminate a proliferating, cohesive mass of cancer cells, ‘tumoroids’ of B16 mouse melanoma cells in non-adhesive culture plates were engineered (FIG. 1A). B16 melanoma is a widely used tumor model for cancer immunotherapy development and does not respond in vivo to T cell checkpoint blockade nor to CD47 disruption and therefore represents a large number of patients thus far unresponsive to immunotherapy. Moreover, because monotherapy with CD47 blockade shows no clinical efficacy against solid tumors, it was important to focus on a cancer line such as B16 that is anticipated to be similarly unaffected by CD47 disruption, rather than study lines already known to respond to CD47 disruption alone (e.g., MC38 colon cancer 27, 28). B16 tumoroid cells adhere to each other and grow as thin, dark cell aggregates with irregular borders (Extended Data FIG. 1A), akin to early-stage melanoma in the upper layer of human skin. 29 Tumoroids resist deformation as a single cohesive mass when pulled into a micropipette (FIG. 1B), with a softness consistent with other neuro-lineage tissues such as brain that also relies for cohesion more on cell-cell adhesion than matrix adhesion. Sustainable stresses of ˜0.5-1 kPa are much higher than those exerted by a macrophage engulfing a microparticle, but tumoroids flow after a few minutes as the stress favors disruption of adhesions—thus illustrating a means by which added macrophages can over time extract B16 cells from the tumoroid. Such viscoelastic behavior is typical of culture-aggregated spheroids, including cell mixtures that sort based on differential adhesion and cortical tension. Indeed, disrupting either Ca2+-dependent cell adhesions or actin polymerization relaxed tumoroids as evidenced by a rapid (<1 h) increase in projected area (FIGS. 9B-9C). Subcutaneous tumors of B16 cells from mice exhibit similar cohesion and softness (FIGS. 9D-9F), despite the presence of various other cell types including macrophages.

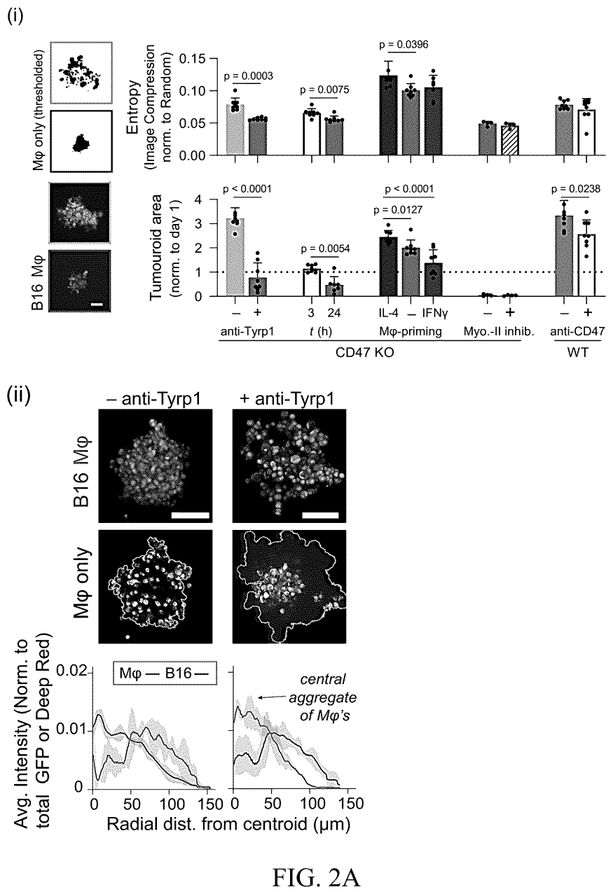

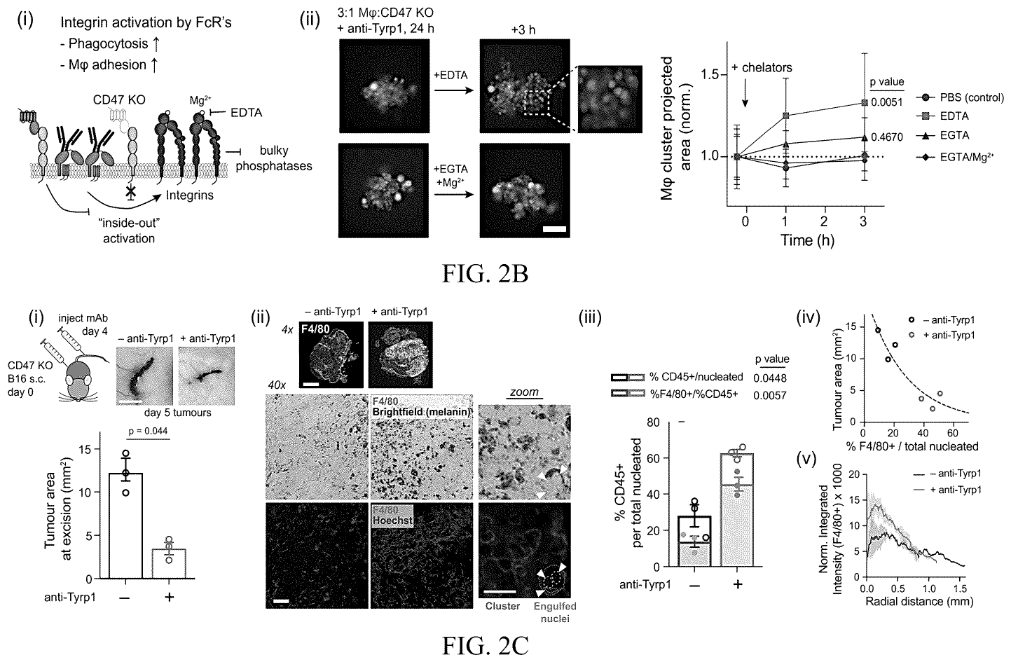

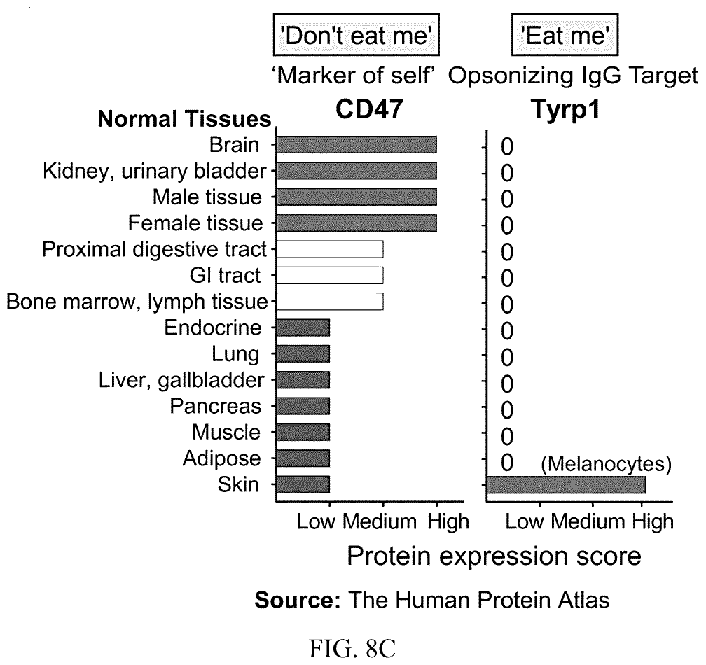

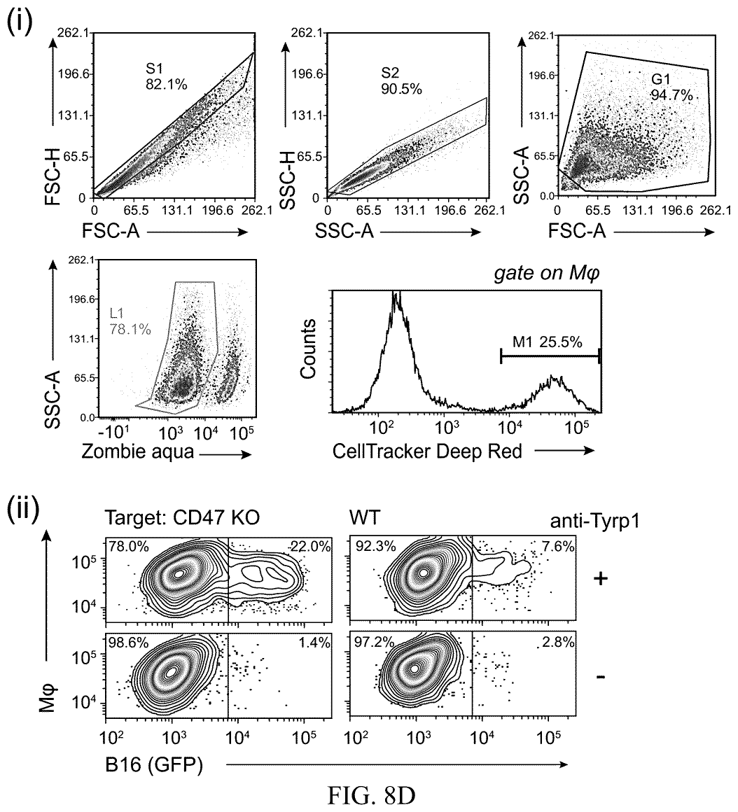

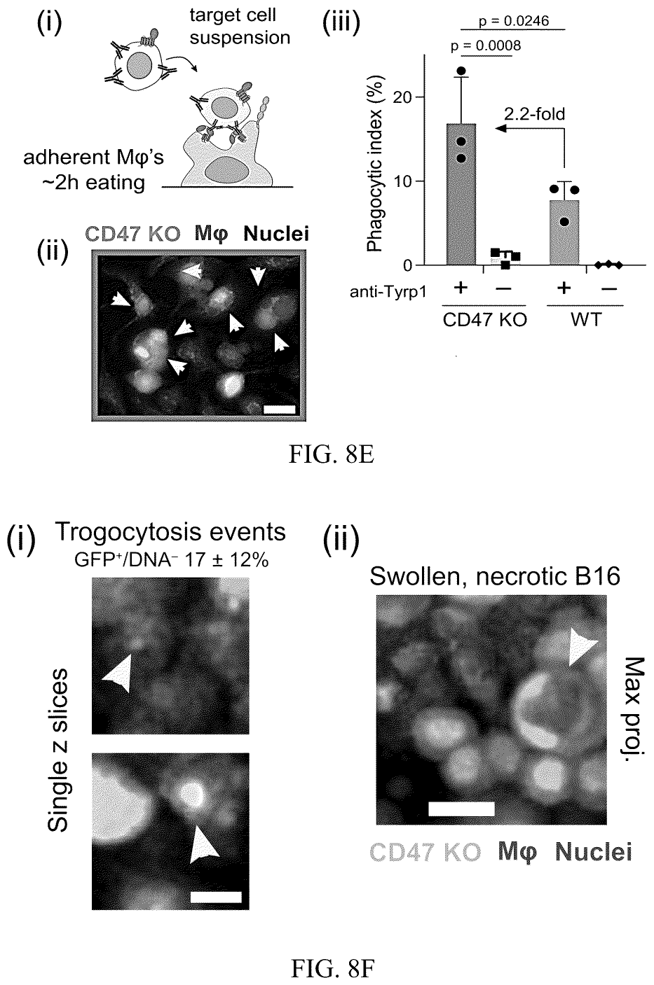

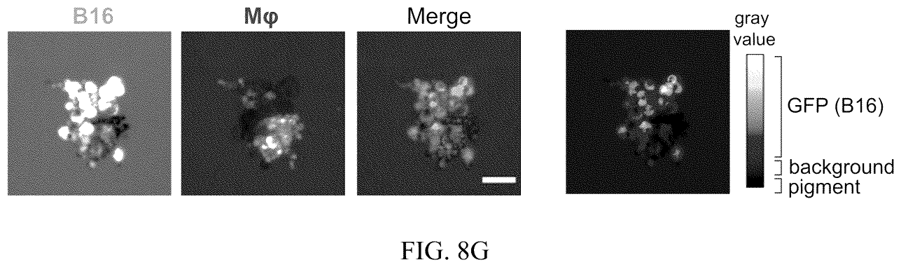

Without wishing to be bound by theory, it was hypothesized that cancer phagocytosis could be maximized for targets lacking CD47, and subsequent studies therefore generated tumoroids from CRISPR/Cas9-engineered B16 cells with either CD47 knockout (KO) or wild-type (WT) levels of CD47 (FIG. 8A). Bone marrow-derived macrophages were added to pre-assembled tumoroids together with anti-Tyrp1 monoclonal antibody that binds and opsonizes B16 cells (FIGS. 8A-8B). Tyrp1 aids in melanin granule synthesis, so that anti-Tyrp1 is relatively specific to melanocytes in contrast to the ubiquitous phagocytosis checkpoint ligand CD47 (FIG. 8C). Despite the specificity, anti-Tyrp1 was recently found ineffective in the clinic against melanoma and shows little to no effect on B16 tumors established in mice. Growth of WT tumoroids is likewise unaffected by anti-Tyrp1 with or without added macrophages, but opsonization of CD47 KO ‘immuno-tumoroids’ maximizes phagocytic macrophages quantified ˜1-day after addition (FIG. 1C and FIG. 8D). Conventional assays with cancer cell suspensions added to immobilized macrophages for 1-2 h show similar phagocytosis trends (FIG. 8E), and despite an important difference elaborated below, the results all suggest macrophages in tumoroids can extract and engulf individual B16 cells, as clearly visualized for opsonized CD47 KO tumoroids (FIG. 1D-i). More than 80% of engulfment events showed co-localization of GFP and DNA, which were considered to represent phagocytosis of an entire cell including its nucleus and cytoplasm. A minority of engulfment events did not stain positively for DNA and might instead represent trogocytosis of cytoplasmic fragments, which could conceivably contribute to tumor cell elimination along with other forms of cell death (Extended Data FIG. 2F). Importantly, only for the maximal phagocytosis condition of CD47 KO and anti-Tyrp1 opsonization is the exponential growth of tumoroids reversed and tumoroids eradicated (FIG. 1D-ii and FIG. 9A).

### Example 2: Macrophages Cooperatively Phagocytose Targets

Surprisingly, macrophages segregate under maximal phagocytosis conditions (FIG. 1D-i). Such a cluster will have few macrophages next to cancer cells and will physically impede interactions with cancer cells compared to the dispersed macrophages under the other conditions. Despite the macrophage clustering, adding more macrophages suppresses growth and fits the calculus of cell proliferation suppressed on a stoichiometric basis by n phagocytes (FIG. 1E and FIG. 9B). The rate constant k_{eff} exhibits a Hill slope n=−1.8 for CD47 KO tumoroids that indicates cooperative phagocytosis as a function of macrophage number (FIG. 9C,9D). WT tumoroids could be eliminated by high concentrations of anti-CD47+anti-Tyrp1 plus high macrophage numbers, and tumoroid area again indicated cooperativity as a function of added macrophages (FIG. 1F-i) but not as a function of anti-CD47 or anti-Tyrp1 (FIG. 1F-ii and Extended Data FIG. 3D-E). High dose anti-CD47 blockade can be difficult to achieve clinically given the low permeation of solid tumors, but high dose opsonization does not seem to be required for tumoroid elimination provided that CD47 signaling is completely disrupted by genetic knockout in target cells as shown here or using engineered macrophages discussed below. The results also underscore a cell-level cooperativity that reflects a cell-level structure such as a macrophage cluster. Indeed, although anti-CD47+anti-Tyrp1 enhanced engulfment of WT B16 cell suspensions in standard 2D phagocytosis assays relative to controls, high-density regions of the immobilized macrophages showed fewer phagocytic macrophages than low-density regions—contrary to cooperativity (FIG. 9F). Tumoroid cohesion might be needed for macrophage cooperativity, with macrophage clusters providing a structural basis for this functional cooperativity—in a hypothetical “tug-of-war” on cohesive tumoroids by macrophages before successful phagocytosis of target cells (FIG. 1G).

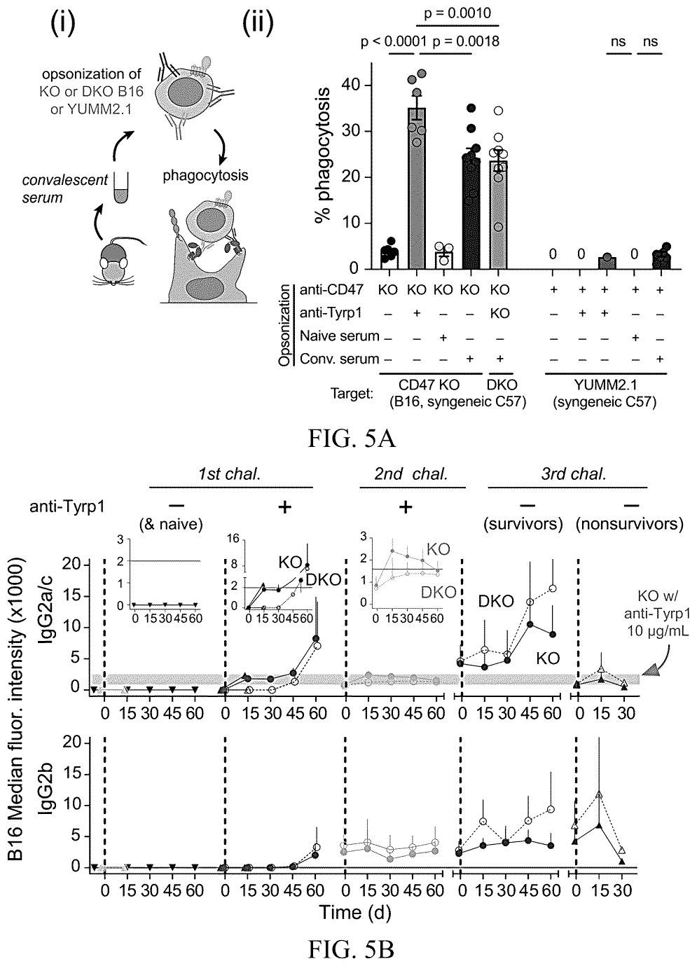

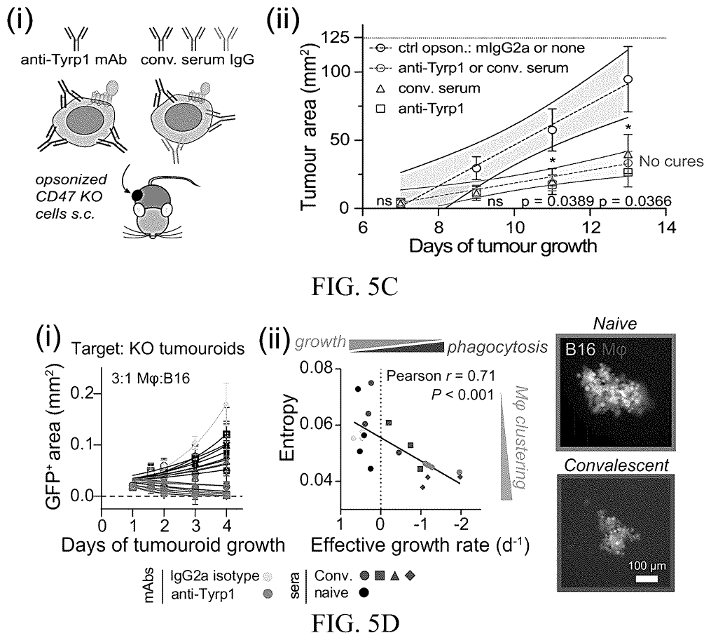

In order to assess whether macrophage clustering consistently associates with tumoroid suppression, an information entropy approach was applied to the many images obtained under a wide range of time points and perturbations. Image files of clustered macrophages are digitally compressed more than images of dispersed macrophages (i.e., more disordered) (FIG. 2A-i and FIG. 10A), and tumoroid area measurements at the same timepoint show the same trend. Cell density profiles further show a central cluster of macrophages displaces B16 cells in tumoroids only under maximum engulfment conditions (FIG. 2A-ii). Kinetic studies indicate clustering takes more than 3 h but is clear by 24 h (FIG. 2A-i). Following tumor cell elimination, macrophages disperse again (FIG. 10B). Tumoroid elimination and tight macrophage clusters are also promoted by pre-treating cultured macrophages with interferon-γ (IFNγ) (FIG. 2A-i), which increases phagocytic surface receptors (FcR's in Extended Data FIG. 5) but also induces a low contractility mechanobiological state consistent with soft tumoroid engulfment (e.g. Myosin-II inhibition has no effect); interleukin-4 (IL-4) has the opposite effects and disperses macrophages. The macrophage priming approach notably avoids direct cytokine effects on B16 growth. Further studies show macrophage clusters are not sufficient for efficient phagocytosis and tumoroid elimination, but the various analyses do indicate that: when macrophages engulf maximally, they cluster.

Cell-cell adhesion for macrophages is mediated by a diversity of inducible integrins, with integrin ligand binding for adhesion depending on Mg2+ and integrin activation occurring downstream of phagocytic FcR signaling when CD47-SIRPα is inhibited. Consistent with FcR expression increased by IFNγ and decreased by IL-4, monocultures of macrophages cluster with IFNγ and disperse with IL-4 with changes in CD47 and SIRPα levels also suggesting net signaling changes (FIG. 11D). To test the effects of such a pathway on clustering (FIG. 2B-i), macrophage clusters engulfing CD47 KO cells (˜24 h) were treated with chelators (EDTA for Mg2+ and Ca2+; EGTA for Ca2+). Only EDTA led to a rapid disaggregation of macrophages (FIG. 2B-ii), which is consistent with the pathway, but EGTA did disrupt B16 aggregates (FIG. 9B), consistent with cadherin adhesion.

### Example 3: Phagocytic Macrophage Density Eliminates Tumors—Engineered Phagocyte Approaches

To assess whether maximization of phagocytic macrophages is effective in vivo, CD47 KO tumors were first established in mice (for 4 days), which then received an intravenous (i.v.) injection of anti-Tyrp1 into the tail-vein (FIG. 2C-i). Just 24 h later, the mAb caused 3-fold smaller tumors and 3-fold more tumor macrophages (F4/80+ cells) versus controls (FIG. 2C-ii-iv). Other immune cells (CD45+, F4/80−) did not differ. Macrophages also clustered and segregated within treated tumors and circumscribed B16 nuclei and melanin (FIG. 2C-ii, v), consistent with macrophage activation for phagocytosis per tumoroids (FIG. 1D and FIG. 8G). Longer duration experiments showed anti-Tyrp1 tail-vein injections eliminated CD47 KO tumors in ˜40% of mice, whereas various control tumors, including untreated or IgG2a isotype and PBS treated CD47 KO tumors, and both treated or untreated WT tumors all showed the expected exponential growth (FIG. 3A-i, ii and FIG. 12A). Partial responders are less frequent with treatment than complete or non-responders (FIG. 3A-ii, inset); this trend seems unusual for cancer therapies, but if phagocytic macrophage numbers vary broadly in vivo across a cohort, then this bimodal response is consistent with tumoroid's showing cooperative-sigmoidal suppression and elimination (FIG. 1E). The lack of a survival benefit of anti-Tyrp1 on established WT tumors is consistent with recent studies.

Previous work established that monocyte-derived macrophages expressing activating FcR's are critical effector cells in subcutaneous B16 tumors treated with anti-Tyrp1 at early time points (i.e. the day of tumor cell inoculation). 40 To begin to assess the effect of increased macrophage numbers and any cooperative effects on established CD47 KO tumors, fresh bone marrow cells (containing 5-10% monocytes and macrophages) were i.v. injected together with anti-Tyrp1. The combination eliminated CD47 KO tumors in ˜80% of mice (FIG. 3A-ii).

Given that WT tumors are unaffected by i.v. injections of anti-CD47 and anti-Tyrp1 (FIG. 3A), it was hypothesized that WT tumors could be eliminated by adding i.v. injections of marrow engineered by addition of anti-SIRPα to block CD47 interactions (A′PB cells in FIG. 3B). This strategy enabled high levels of phagocytosis and eliminated tumoroids in vitro at high macrophage:B16 ratios (FIG. 3C-i, FIG. 12B). Although this particular anti-SIRPα enhanced phagocytosis of IgG-opsonized RBCs but had no effect with unopsonized cancer cells, this mAb does not directly block CD47 interactions but instead immobilizes SIRPα to prevent its accumulation in the phagocytic synapse (FIG. 12D). The A′PB approach indeed gave complete responses in ˜10% of mice with WT tumors (FIG. 12C)—which increased to ˜17% with anti-CD47 injections and to ˜38% with a second i.v. injection of the engineered marrow cells (FIG. 3C-ii). Other recent studies with injection of fluorescent A′PB showed regression of tdTomato-expressing, opsonized tumors in NSG mice, and A′PB cells in these tumors were activated for phagocytosis based on tdTomato uptake, consistent with images of B16 engulfment here (FIG. 2C-ii), and also showed gene expression consistent with a classically phagocytic sub-type.

To confirm macrophages are the effector cells and to improve upon transient antibody based SIRPα blockade with a genetic engineering approach, SIRPα was deleted in conditionally immortalized macrophage (CIM) progenitors that retain macrophage differentiation ability and phenotypes (FIG. 13A) (gating strategy illustrated in FIG. 17A). These engineered macrophages, SIRPα-KO CIM, also readily phagocytosed target B16 cells with anti-Tyrp1 opsonization in vitro (FIG. 3D-i). Tail-vein injection of SIRPα-KO CIM's with anti-Tyrp1 (but no added anti-CD47) produced complete anti-tumor responses against WT tumors in ˜60% of mice (FIG. 3D-ii). Thus, as disclosed herein with the immuno-tumoroid results, a succinct summary of many experiments shows that maximizing phagocytosis is necessary for tumor elimination and high numbers of phagocytic macrophages maximize efficacy (FIG. 3E and FIG. 12E).

Macrophages and related phagocytic immune cells provide just a first line of defense but often initiate acquired immunity. Therefore, surviving mice were challenged with a second injection of CD47 KO cells 80 days after the initial challenge and again treated with anti-Tyrp1 (a prime-boost strategy for an anti-cancer vaccine). From the initial cohort in which the complete response rate was about 40% (FIG. 3A-ii), about 80% of the re-challenged mice survived (FIG. 4A). Age-matched naïve mice receiving their first challenge with or without treatment responded similarly to the younger cohorts. A third tumor challenge was left untreated (i.e., no anti-Tyrp1), and 75% again resisted tumor growth, which indicates a durable immunological memory. Whitening of the melanized fur was evident across all three challenges of treated mice in the KO cohorts, consistent with an immune response against normal melanocytes that occurs over time with anti-Tyrp1 as mediated by Fc- or complement receptors. A second model of B16 tumors involving metastasis to lung with CD47 KO cells showed anti-Tyrp1 prolongs survival of naïve mice until all mice succumb with clinical symptoms (FIG. 4B). Importantly, however, mice that previously showed complete responses against subcutaneous tumors could achieve a complete anti-tumor response against lung metastasis when treated with anti-Tyrp1, as demonstrated by long-term survival of ˜35% of mice.

Although the increased survival rates with engineered macrophages indicate these are key effector cells, tumor growth was assessed in NSG mice that lack all adaptive immunity (no antibodies and no T cells, B cells, or NK cells) but have macrophages which display suitable Fc-receptors. Growth of CD47 KO B16 tumors proves similar in NSG mice to growth in C57 mice, and anti-Tyrp1 i.v. injections again slowed the growth of tumors in NSG mice by up to 50% (FIG. 4C). Wild-type B16 tumors are similarly suppressed by SIRPα-KO CIMs treatments (FIG. 4D). Such results underscore the key initiating role of macrophages as effector cells in the B16 tumor suppression.

### Example 4: Convalescent Serum IgG Drives Tumor Phagocytosis and Macrophage Clustering

Despite the importance of anti-Tyrp1 for tumor elimination in immunocompetent C57, this monoclonal antibody was not used in a third challenge of CD47 KO tumors cells in which 75% of mice resisted the tumor (FIG. 4A). Without wishing to be bound by theory, it was hypothesized therefore that an acquired immune response is generated that might also extend beyond Tyrp1. Subsequent studies thus knocked out Tyrp1 in the CD47 KO cells (double knockout, DKO) and engrafted these DKO cells in complete responders from the initial CD47 KO treatment cohort. It was found that without monoclonal anti-Tyrp1 treatment, which would not be effective against DKO tumors anyway, 50% of mice survived (FIG. 4E-i, ii).

Serum collected throughout tumor challenge and treatment experiments was used to immunoblot B16 lysates, which revealed an increasing number of bands with progression of challenges in support of the hypothesis of immune responses against B16 antigens beyond Tyrp1 (FIG. 4F). Additionally, convalescent serum from complete responder mice was found to bind to WT B16 cells (FIG. 18A). To test whether such serum antibodies are functional to promote phagocytosis, serum from various mice was added to CD47 KO and DKO B16 cells and then added this mixture to macrophages in culture. Most convalescent sera increased phagocytosis relative to naïve serum (FIGS. 5A and 18B), with similar results for CD47 KO, DKO, and WT B16 parental cells as targets for phagocytosis (FIGS. 13B, 13C, and 17B). As expected, anti-Tyrp1 does not drive phagocytosis of DKO cells, and neither anti-Tyrp1 nor the serum from complete responders has any effect on engulfment of YUMM2.1 melanoma cells that were also derived from mice on the C57 background (FIGS. 5A and 18C). Convalescent serum antibodies thus target B16-specific antigens beyond Tyrp1 but not antigens expressed by a syngeneic melanoma or xenogeneic antigens present on cultured cell lines (e.g., bovine antigens from media containing fetal bovine serum). Assays of IgG subclasses confirm the presence of pro-phagocytic IgG2a/c and IgG2b that bind CD47 KO cells and DKO cells after second and third challenges (FIG. 5B), with other IgG subtypes also detected (FIG. 14A-14C). Further consistent with a progressive prime-boost vaccination and antigenic spread beyond Tyrp1, survivors of the third B16 challenge (with no monoclonal anti-Tyrp1 injected) showed higher levels of IgG2a/c compared to non-survivors.

Definitive de novo anti-B16 IgG's that bind DKO cells were detected in serum well after the first challenge (˜day 45 in FIG. 5B). Immunocompromised NSG mice do not survive this long even with injection of SIRPα-KO CIMs (FIG. 4C), suggesting that other acquired immune cells (e.g., T cells) contribute to survival even before IgG is detectable. Nonetheless, anti-B16 IgG conceivably contribute to acquired immunity by opsonizing tumor cells and by antigen presentation among other effector functions. To test the function of convalescent serum in vivo, CD47 KO B16 cells were opsonized with the serum just prior to subcutaneous implantation in naïve mice (FIG. 5C). Tumor growth was suppressed by days 11 and 13 similar to anti-Tyrp1 and relative to controls. However, because any pre-bound IgG will be diluted by B16 proliferation or else lost by dissociation, subsequent studies assessed complete elimination of tumor cells by convalescent serum and macrophages with tumoroids.

Convalescent serum IgG added with macrophages to CD47 KO tumoroids eliminated tumoroids for the most potent samples (FIG. 5D-i). Importantly, serum activity against tumoroids correlated with macrophage clustering at day 2 similar to anti-Tyrp1 (FIG. 5D-ii), consistent with effects of monoclonal anti-Tyrp1 (FIG. 2A). Serum alone had no effect (FIG. 14D), indicating macrophages are the effector cells for the anti-B16 serum IgG.

### Example 5: CD47-SIRPα Disruption Plus IgG Opsonization in Other Immunocompetent Tumor Models

To begin to generalize some of the findings from the extensive studies presented herein with B16, the RM-9 prostate cancer model was used and targeted the clinically relevant tumor antigen GD2, a ganglioside-lipid on multiple tumor types. Combination of FcR-engaging anti-GD2 mAbs with anti-CD47 recently showed suppression of neuroblastomas and osteosarcomas in immunodeficient NSG mice (with little effect in monotherapies) and in one syngeneic neuroblastoma in immuno-dysfunctional 129X1/SvJ mice. In immunocompetent C57 mice, IgG3 anti-GD2 that is otherwise ineffective has been combined with cytokines to suppress RM-9 tumors. The IgG2a anti-GD2 used here strongly promoted engulfment of RM-9's only in combination with anti-CD47, and exponential growth of established RM-9 tumors in C57 mice (which is similar to B16 tumors; FIG. 3A) was suppressed in some mice by tail-vein injections of SIRPα-KO CIMs plus anti-GD2 (FIG. 15A). In fully immunocompetent models, this is the first in vivo success with opsonizing anti-GD2 combined with CD47-SIRPα disruption—particularly with an engineered cell therapy. TC-1 mouse lung cancer cells with complete knockout of CD47 were also assessed, but a tumor-specific opsonizing mAb is lacking—and a major challenge for many cancers in mice and in the clinic—so TC-1 cells were opsonized with a polyclonal IgG raised against mouse-RBCs that works in RBC phagocytosis and more generally with mouse cells. Pre-opsonization of TC-1 CD47 KO suppressed initial tumor growth in vivo (FIG. 15B), analogous to B16 studies (FIG. 5C). Tumor-specific IgG were also sought in C57 mice that survived CT-2A glioma tumors because of vaccine-enhanced CAR-T therapy, but serum did not promote phagocytosis even though all murine-IgG subclasses bound to CT-2A's (FIG. 15C). Binding of Ab's far from the cell surface are known to be inefficient in phagocytosis. Regardless, anti-cancer IgG's in survivors from a T cell therapy could differ in key functional ways from IgG's induced via a macrophage therapy.

### Example 6: Selected Discussion

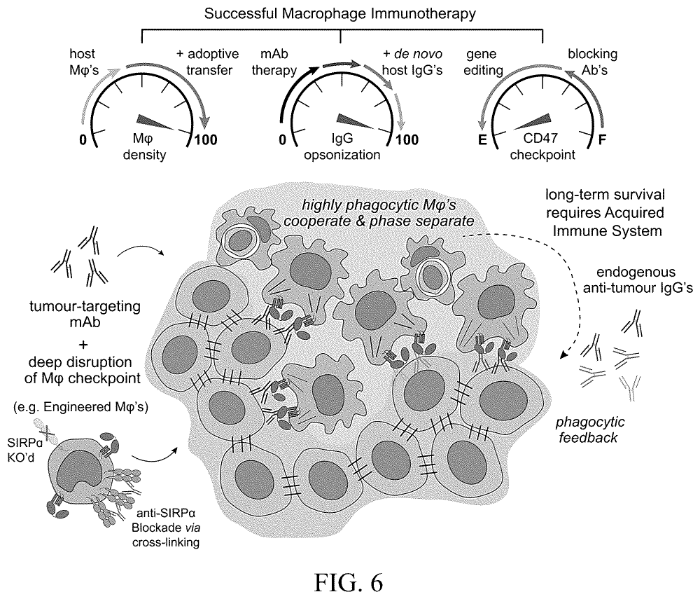

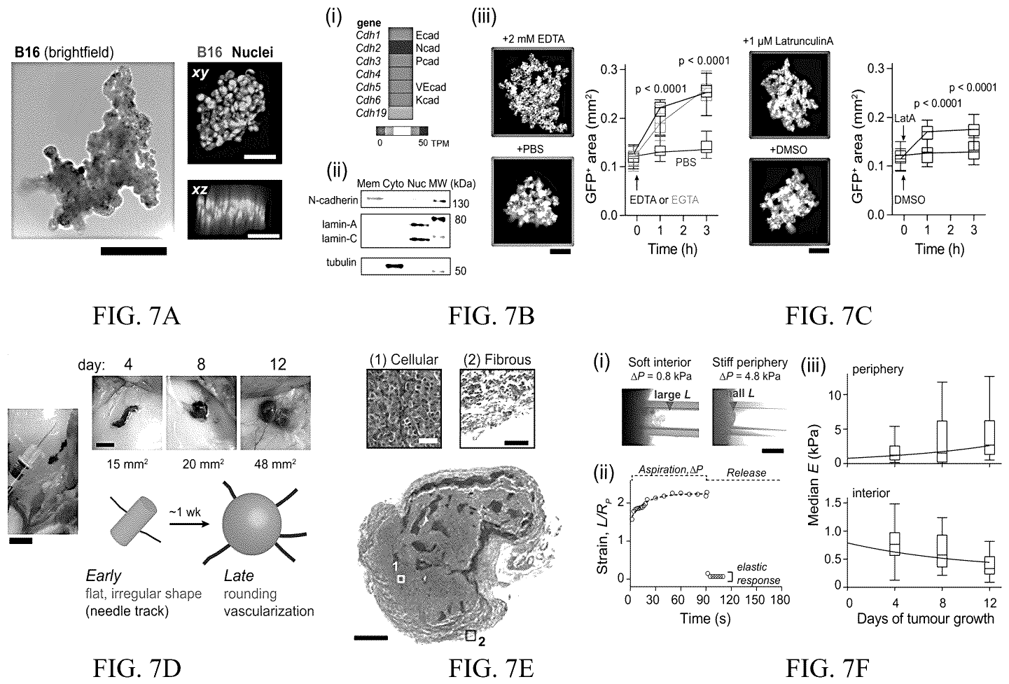

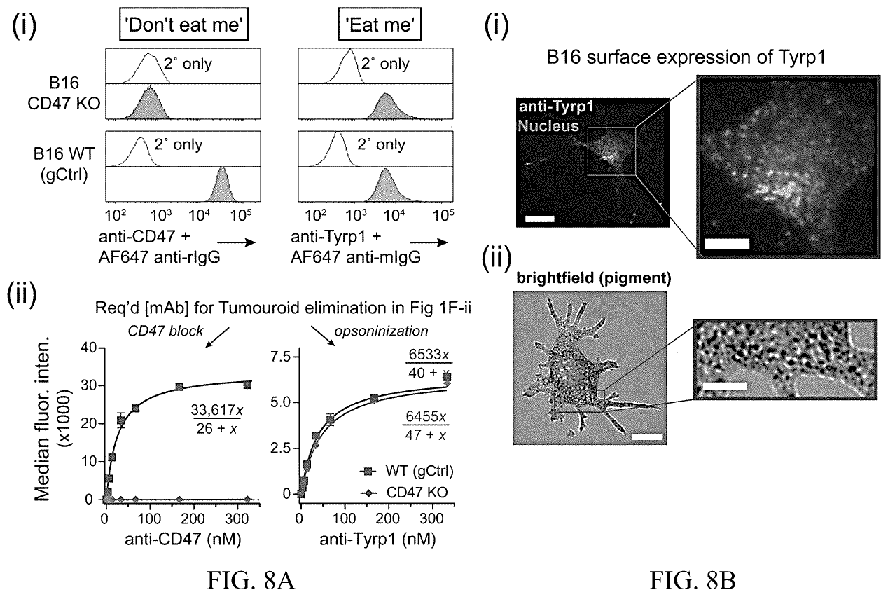

Macrophage immunotherapy of solid tumors benefits from maximizing three factors in combination: first is macrophage number that can lead to cooperativity, second is tumor opsonization that activates Fc receptors, and third is disruption of the macrophage checkpoint (FIG. 6). Immunocompetent mice that survive develop de novo anti-tumor IgG that are pro-phagocytic and pro-clustering. This might be a first for tumors, but immunodeficient NOD mice develop anti-RBC IgG after CD47 knockout and die with anemia, which suggests IgG-opsonization function. Various effector functions for the anti-cancer IgG are possible in vivo and require further study, but their isolation and cloning (from mouse or eventually patients) could help address a need for IgG's that combine with clinically relevant CD47 blockade for anti-tumor therapy—including resistance with antigen loss. The approach disclosed herein is poised to take advantage of biopharmaceutical expertise in therapeutic mAbs and new vaccine technologies (e.g., mRNA) to produce tumor-specific IgG's.

The most promising clinical application of CD47-SIRPα blockade to date combines anti-CD47 (magrolimab) and anti-CD20 (rituximab) against a liquid tumor, non-Hodgkin's lymphoma, but rituximab depletion of B cells prevents development of anti-tumor IgG and any phagocytic feedback. Anti-cancer IgG in cancer patients include anti-(human Tyrp1) from a melanoma patient, but anti-Tyrp1 lacks clinical efficacy against melanoma. High TYRP1 in metastatic melanoma associates with poor survival (FIG. 16A), suggesting Tyrp1 is targetable, and although low CD47 associates with poor survival, even moderate CD47 levels are protective (FIG. 1F-ii). Importantly, engineered macrophages can enhance via their function and their numbers the efficacy of otherwise ineffective Abs. For the many melanoma patients unresponsive to current therapies, results here specifically suggest anti-Tyrp1 be combined with deep CD47-SIRPα disruption plus phagocytic macrophages; no such trials are currently registered, and serum could yield new anti-melanoma IgG.

CD47 deletion on its own has no effect on B16 tumors in immunocompetent mice (FIG. 3A), which aligns well with monotherapy with anti-CD47 showing no efficacy in the clinic, particularly against solid tumors. In contrast, some mouse-engrafted tumors show CD47 monotherapy is efficacious; one pair of studies shows anti-CD47 or knockout are effective on their own against liquid and solid tumors (i.e., MC38 colon) while ruling out any role for macrophages and showing T cell depletion abrogates any suppression. Relevance to human patients is now questionable and likely attributable to high immunogenicity of the tumors; compared to B16 cells, many tumor lines (e.g., MC38 colon) are indeed more immunogenic in immunocompetent mice (FIG. 16B). Importantly, RM-9 prostate cells are poorly immunogenic like B16 cells and show similarly rapid growth (Extended Data FIG. 9A) which anti-correlates with immunogenicity (FIG. 16B). Numerous immune cell types and factors likely contribute to suppression of patient-relevant B16 tumors and could be perturbed or depleted in future studies, including studies of T cells, B cells, and especially dendritic cells that are often phagocytic and of course a macrophage-related myeloid lineage implicated in acquired immunity. Nonetheless, the two approaches with suitably engineered macrophages maximize survival (FIG. 3B-E) even in the absence of any acquired immune cells (FIG. 4D)—indicating a key initiating role for phagocytic macrophages. Such cells also accumulate early in tumors and engulf, while other immune cell types (CD45+, F4/80−) remain overall constant (FIG. 2C).

Tumor-associated macrophages are generally not phagocytic but are often abundant and a poor prognosis in human tumors including melanoma. However, phagocytic macrophage clusters in thyroid cancer associate with decreased risk of metastasis, and a negative correlation between macrophage abundance and survival in follicular lymphoma patients was potentially reversed with rituximab. Critical for immunotherapies is a deeper understanding of tumor microenvironment biophysics, including how various IgG's diffuse and function or how immune cells infiltrate and interact. In particular, sub-saturating doses of opsonizing anti-Tyrp1 is highly effective against CD47-disrupted targets, whereas deep disruption of CD47 or SIRPα is necessary for efficacy (FIG. 1-Fii). SIRPα knockout CIM progenitors indeed showed better efficacy than anti-SIRPα engineered macrophages (FIG. 3E). Anti-SIRPα will dissociate, but inhibition via anti-SIRPα crosslinking (FIG. 12D) is also a surprising biophysical mechanism that suggests a new class of crosslinkers. Nonetheless, limits to passive permeation of opsonizing and blocking antibodies are potentially overcome with macrophages engineered here and elsewhere, because they can actively permeate through tight spaces and potentially contribute to acquired immunity and an emergent phagocytic feedback.

### ENUMERATED EMBODIMENTS

The following enumerated embodiments are provided, the numbering of which is not to be construed as designative levels of importance.

Embodiment 1 provides a modified phagocytic cell or precursor cell thereof comprising:
a. an isolated nucleic acid encoding a fusion protein comprising an estrogen receptor (ER) ligand binding domain linked to a Hoxb8 protein,
b. a modified endogenous genetic locus encoding a SIRPα protein; and
c. Fc-γR receptors bound to at least one targeting antibody specific for an antigen on a target cell.

Embodiment 2 provides the modified phagocytic cell or precursor cell thereof of embodiment 1, wherein the phagocytic cell is a macrophage.

Embodiment 3 provides the modified phagocytic cell or precursor cell thereof of embodiment 1, wherein the precursor cell is a monocyte precursor cell.

Embodiment 4 provides the modified phagocytic cell or precursor cell thereof of embodiment 1, wherein the modified SIRPα locus reduces or eliminates expression of SIRPα protein.

Embodiment 5 provides the modified phagocytic cell or precursor cell thereof of embodiment 1, wherein the target cell antigen is a tumor-associated antigen.

Embodiment 6 provides the modified phagocytic cell or precursor cell thereof of embodiment 1, wherein the target cell is a tumor cell.

Embodiment 7 provides the modified phagocytic cell or precursor cell thereof of embodiment 1, wherein the isolated nucleic acid is introduced into the phagocytic cell or precursor cell thereof via a lentiviral transduction system.

Embodiment 8 provides the modified phagocytic cell or precursor cell thereof of embodiment 1, wherein the modified locus encoding a SIRPα protein is modified via a CRISPR system.

Embodiment 9 provides the modified phagocytic cell or precursor cell thereof of embodiment 1, wherein the nucleic acid encoding the ER-Hoxb8 protein is introduced prior to the modification of the SIRPα protein.

Embodiment 10 provides the modified phagocytic cell or precursor cell thereof of embodiment 1, wherein expression of the ER-Hoxb8 fusion protein reversibly or conditionally immortalizes the phagocytic cell or precursor cell thereof when the cell is contacted with a ligand of the estrogen receptor.

Embodiment 11 provides the modified phagocytic cell or precursor cell thereof of embodiment 1, wherein the macrophage or precursor cell thereof is of human origin.

Embodiment 12 provides a method of producing a modified phagocytic cell or precursor cell thereof, comprising:
a. obtaining a precursor cell from a subject;
b. transducing the precursor cell with a expression vector encoding a fusion protein comprising an estrogen receptor ligand binding domain and a Hoxb8 protein;
c. contacting the precursor cell with a estrogen receptor ligand to reversibly immortalize the precursor cell;
d. modifying an endogenous genetic locus encoding a SIRPα protein;
e. terminally differentiating the precursor cell into a phagocytic cell by withdrawal of the ER ligand and culture in the presence of an effective amount of stem cell factor (SCF) and granulocyte-macrophage colony-stimulating factor (GM-CSF); and
f. coating the Fc receptors of the phagocytic cell with at least one targeting antibody specific for an antigen on a target cell.

Embodiment 13 provides the method of embodiment 12, wherein the modification to the genetic locus encoding SIRPα reduces or eliminates expression of SIRPα protein.

Embodiment 14 provides the method of embodiment 12, wherein the genetic modification is accomplished using a CRISPR knockout system.

Embodiment 15 provides the method of embodiment 12, wherein the precursor cell is a monocyte precursor cell.

Embodiment 16 provides the method of embodiment 12, wherein the phagocytic cell is a macrophage.

Embodiment 17 provides the method of embodiment 12, wherein the expression vector is a lentiviral vector.

Embodiment 18 provides the method of embodiment 12, wherein the precursor cell is obtained from bone marrow.

Embodiment 19 provides the method of embodiment 12, wherein the estrogen receptor ligand is β-estradiol.

Embodiment 20 provides the method of embodiment 12, wherein the target cell is a tumor cell.

Embodiment 21 provides the method of embodiment 12, wherein the target cell antigen is a tumor-associated antigen.

Embodiment 22 provides the method of embodiment 12, wherein the subject is a mammal.

Embodiment 23 provides the method of embodiment 12, wherein the subject is a human.

Embodiment 24 provides a method of treating cancer in a subject in need thereof, comprising administering to the subject an effective amount of the modified phagocyte or precursor cell thereof of any one of embodiments 1-11 thereby treating the cancer, wherein the targeting antibody is specific for at least one antigen associated with the cancer.

Embodiment 25 provides the method of embodiment 24, further comprising administering to the patient an effective amount of an opsonin specific for the cancer.

Embodiment 26 provides the method of embodiment 25, wherein the opsonin is the targeting antibody.

Embodiment 27 provides the method of embodiment 25, wherein the opsonin is different from the targeting antibody.

Embodiment 28 provides the method of embodiment 24, wherein the subject is a mammal.

Embodiment 29 provides the method of embodiment 24, wherein the subject is a human. Embodiment 30 provides the method of embodiment 24, wherein the cancer is selected from the group consisting of brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, and uterine cancer.

Embodiment 31 provides the method of embodiment 24, wherein the cancer is melanoma.

Embodiment 32 provides the method of embodiment 24, wherein the target cell antigen is Tyrp1.

Embodiment 33 provides a composition comprising the modified phagocyte or precursor cell thereof of any one of embodiments 1-11 and a pharmaceutically acceptable carrier or excipient.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

What is claimed is:

1. A modified phagocytic cell or precursor cell thereof comprising:
a. an isolated nucleic acid encoding a fusion protein comprising an estrogen receptor (ER) ligand binding domain linked to a Hoxb8 protein,
b. a modified endogenous genetic locus encoding a SIRPα protein; and
c. Fc-γR receptors bound to at least one targeting antibody specific for an antigen on a target cell.

2. The modified phagocytic cell or precursor cell thereof of claim 1, wherein the phagocytic cell is a macrophage.

3. The modified phagocytic cell or precursor cell thereof of claim 1, wherein the precursor cell is a monocyte precursor cell.

4. The modified phagocytic cell or precursor cell thereof of claim 1, wherein the modified SIRPα locus reduces or eliminates expression of SIRPα protein.

5. The modified phagocytic cell or precursor cell thereof of claim 1, wherein the target cell antigen is a tumor-associated antigen.

6. The modified phagocytic cell or precursor cell thereof of claim 1, wherein the target cell is a tumor cell.

7. The modified phagocytic cell or precursor cell thereof of claim 1, wherein the isolated nucleic acid is introduced into the phagocytic cell or precursor cell thereof via a lentiviral transduction system.

8. The modified phagocytic cell or precursor cell thereof of claim 1, wherein the modified locus encoding a SIRPα protein is modified via a CRISPR system.

9. The modified phagocytic cell or precursor cell thereof of claim 1, wherein the nucleic acid encoding the ER-Hoxb8 protein is introduced prior to the modification of the SIRPα protein.

10. The modified phagocytic cell or precursor cell thereof of claim 1, wherein expression of the ER-Hoxb8 fusion protein reversibly or conditionally immortalizes the phagocytic cell or precursor cell thereof when the cell is contacted with a ligand of the estrogen receptor.

11. The modified phagocytic cell or precursor cell thereof of claim 1, wherein the macrophage or precursor cell thereof is of human origin.

12. A method of producing a modified phagocytic cell or precursor cell thereof, comprising:
a. obtaining a precursor cell from a subject;
b. transducing the precursor cell with an expression vector encoding a fusion protein comprising an estrogen receptor ligand binding domain and a Hoxb8 protein;
c. contacting the precursor cell with a estrogen receptor ligand to reversibly immortalize the precursor cell;
d. modifying an endogenous genetic locus encoding a SIRPα protein;
e. terminally differentiating the precursor cell into a phagocytic cell by withdrawal of the ER ligand and culture in the presence of an effective amount of stem cell factor (SCF) and granulocyte-macrophage colony-stimulating factor (GM-CSF); and
f. coating the Fc receptors of the phagocytic cell with at least one targeting antibody specific for an antigen on a target cell.

13. The method of claim 12, wherein the modification to the genetic locus encoding SIRPα reduces or eliminates expression of SIRPα protein.

14. The method of claim 12, wherein the genetic modification is accomplished using a CRISPR knockout system.

15. The method of claim 12, wherein the precursor cell is a monocyte precursor cell.

16. The method of claim 12, wherein the phagocytic cell is a macrophage.

17. The method of claim 12, wherein the expression vector is a lentiviral vector.

18. The method of claim 12, wherein the precursor cell is obtained from bone marrow.

19. The method of claim 12, wherein the estrogen receptor ligand is β-estradiol.

20. The method of claim 12, wherein the target cell is a tumor cell.

21. The method of claim 12, wherein the target cell antigen is a tumor-associated antigen.

22. The method of claim 12, wherein the subject is a mammal.

23. The method of claim 12, wherein the subject is a human.

24. A method of treating cancer in a subject in need thereof, comprising administering to the subject an effective amount of the modified phagocyte or precursor cell thereof of claim 1 thereby treating the cancer, wherein the targeting antibody is specific for at least one antigen associated with the cancer.

25. The method of claim 24, further comprising administering to the patient an effective amount of an opsonin specific for the cancer.

26. The method of claim 25, wherein the opsonin is the targeting antibody.

27. The method of claim 25, wherein the opsonin is different from the targeting antibody.

28. The method of claim 24, wherein the subject is a mammal.

29. The method of claim 24, wherein the subject is a human.

30. The method of claim 24, wherein the cancer is selected from the group consisting of brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, gastric cancer, pancreatic cancer, renal cancer, skin cancer, thymoma, sarcoma, non-Hodgkin's lymphoma, Hodgkin's lymphoma, and uterine cancer.

31. The method of claim 24, wherein the cancer is melanoma.

32. The method of claim 24, wherein the target cell antigen is Tyrp1.

33. A composition comprising the modified phagocyte or precursor cell thereof of claim 1 and a pharmaceutically acceptable carrier or excipient.
